(19) United States
(12) Patent Application Publication
(10) Pub. No.: US 2026/0108288 A1
(43) Pub. Date: Apr. 23, 2026

(54) PULSED FIELD ABLATION APPARATUS AND RELATED METHODS

(71) Applicant: AtriCure, Inc., Mason, OH (US)

(72) Inventors: William Dannaher, Bethel, OH (US); Jackson Thomas Romelli, Cincinnati, OH (US); Jeffrey C. Gagel, Mason, OH (US); Kurtis Klingenberg, Mason, OH (US)

(73) Assignee: AtriCure, Inc., Mason, OH (US)

(21) Appl. No.: 19/223,132

(22) Filed: May 30, 2025

Related U.S. Application Data
(60) Provisional application No. 63/653,756, filed on May 30, 2024.

(51) Int. Cl. A61B 18/00; A61B 90/00

(52) U.S. Cl. CPC A61B 18/00; A61B 90/06; A61B 2018/00083; A61B 2018/0016; A61B 2018/00184; A61B 2018/00577; A61B 2018/00642; A61B 2018/00767; A61B 2018/0091

(57) ABSTRACT

Methods and devices for carrying out pulsed field ablation are disclosed. Specifically, the instant disclosure includes surgical clamps configured to clamp tissue and determine the distance between repositionable jaws and tailor pulse field ablation energy to the thickness of the clamped tissue, which is indicative of the spacing between the repositionable jaws. Also disclosed are methods of calibrating pulse field ablation devices and methods of using pulse field ablation devices.

### CROSS-REFERENCE TO RELATED APPLICATIONS

The present application claims the benefit of U.S. Provisional Patent Application No. 63/653,756, filed on May 30, 2024 and titled “PFA CLAMP: JAW CROSS SECTION AND DYNAMIC FIRING SEQUENCES,” and U.S. Provisional Patent Application No. 63/655,930, filed on Jun. 4, 2024 and titled “PFA CLAMP: DOSE ALGORITHM DETERMINATION VIA JAW DISTANCE SENSING VIA CONTINUITY SWITCHES,” and U.S. Provisional Patent Application No. 63/656,001, filed on Jun. 4, 2024 and titled “DOSE ALGORITHM DETERMINATION VIA JAW DISTANCE SENSING VIA HALL EFFECTS SENSOR,” and U.S. Provisional Patent Application No. 63/655,982, filed on Jun. 4, 2024 and titled “DOSE ALGORITHM DETERMINATION VIA JAW DISTANCE SENSING THROUGH MEASUREMENT OF SEALED LINEAR MEMBRANE POTENTIOMETER,” the disclosure of each of which is hereby incorporated by reference in its entirety.

### INTRODUCTION TO THE INVENTION

The present disclosure is directed to ablation devices, and, more specifically, to ablation systems and devices configured to perform pulsed field ablation, components thereof, and related methods.

The present disclosure contemplates that ablation systems configured to perform pulsed field ablation (“PFA”) may be used in various medical and surgical procedures. Generally, PFA systems may be used to ablate targeted cells while limiting potential collateral damage to non-targeted tissues. PFA typically involves applying high-voltage electrical pulses to a target tissue. The pulses create high-intensity electrical fields, which disrupt the integrity of the cell membranes in the target tissue. As a result, over a short period of time (e.g., days to weeks), the cells die, creating a lesion in the target tissue.

The present disclosure contemplates that PFA may be used for ablation of cardiac tissue for treatment of cardiac arrhythmias. Some known PFA technologies, such as catheter-based devices, may produce sub-optimal results in some circumstances. For example, the present disclosure contemplates that it may be difficult to maintain desired contact pressure between catheter-based devices and an interior heart wall, thus potentially resulting in lesions that may be inaccurately positioned and/or less durable than desired.

While known PFA systems have been used to perform some cardiac ablation procedures, particularly endocardial ablations (e.g., on the inside surface of the heart), improvements in the construction and operation of PFA systems and PFA devices may be beneficial for users (e.g., physicians and surgeons) and patients. The present disclosure includes various improvements that may enhance the construction, operation, and use of PFA systems and PFA devices, including embodiments applicable to epicardial ablation (e.g., on the outside surface of the heart and/or penetrating the tissue surface).

It is a first aspect of the present invention to provide a a surgical device, comprising: (i) a shaft; and (ii) a first jaw and a second jaw operatively coupled to a distal end of the shaft, wherein the first jaw is repositionable with respect to the second jaw, where each of the first jaw and the second jaw comprises at least three electrodes exposed with respect to an insulating material and spaced radially across a longitudinal cross-section thereof, and where each of the first jaw and the second jaw terminates at a respective tip.

In a more detailed embodiment of the first aspect, the longitudinal cross-section of the first jaw includes the insulating material exhibiting a convex profile, and the longitudinal cross-section of the second jaw includes the insulating material exhibiting a convex profile. In yet another more detailed embodiment, the at least three electrodes of the first jaw are equidistantly spaced radially across the longitudinal cross-section thereof, and the at least three electrodes of the second jaw are equidistantly spaced radially across the longitudinal cross-section thereof. In a further detailed embodiment, each of the at least three electrodes of the first jaw is configured to overlap another of each of the at least three electrodes of the second jaw. In still a further detailed embodiment, the longitudinal cross-section of the first jaw includes the insulating material exhibiting a convex arcuate profile, and the longitudinal cross-section of the second jaw includes the insulating material exhibiting a convex arcuate profile. In a more detailed embodiment, at least one of the at least three electrodes extends from an apex of the convex arcuate profile of the first jaw, and at least one of the at least three electrodes extends from an apex of the convex arcuate profile of the second jaw. In a more detailed embodiment, at least one or two of the at least three electrodes is recessed with respect to an apex of the convex arcuate profile of the first jaw, and at least one or two of the at least three electrodes is recessed with respect to an apex of the convex arcuate profile of the second jaw. In another more detailed embodiment, the longitudinal cross-section of the first jaw includes the insulating material exhibiting a convex profile, and the longitudinal cross-section of the second jaw includes the insulating material exhibiting a concave profile. In yet another more detailed embodiment, the at least three electrodes of the first jaw are equidistantly spaced radially across the longitudinal cross-section thereof, and the at least three electrodes of the second jaw are equidistantly spaced radially across the longitudinal cross-section thereof. In still another more detailed embodiment, each of the at least three electrodes of the first jaw is configured to overlap another of each of the at least three electrodes of the second jaw.

In yet another more detailed embodiment of the first aspect, at least one of the first jaw and the second jaw includes a vacuum port configured to be in fluid communication with a vacuum source. In yet another more detailed embodiment, the second jaw includes the vacuum port configured to be in fluid communication with the vacuum source, the vacuum port includes a plurality of vacuum ports, and the plurality of vacuum ports interpose the at least three electrodes. In a further detailed embodiment, the longitudinal cross-section of the first jaw includes the insulating material exhibiting an arcuate convex profile, and the longitudinal cross-section of the second jaw includes the insulating material exhibiting an arcuate concave profile. In still a further detailed embodiment, a radius of curvature of the arcuate convex profile is less than a radius of curvature of the arcuate concave profile. In a more detailed embodiment, a radius of curvature of the arcuate convex profile is equal to a radius of curvature of the arcuate concave profile. In a more detailed embodiment, a radius of curvature of the arcuate convex profile is greater than a radius of curvature of the arcuate concave profile. In another more detailed embodiment, the longitudinal cross-section of the second jaw includes the insulating material exhibiting a concave arcuate profile with opposed lateral rims, and the at least three electrodes are recessed below a height of the opposed lateral rims. In yet another more detailed embodiment, the longitudinal cross-section of the first jaw includes the insulating material exhibiting a concave profile, and the longitudinal cross-section of the second jaw includes the insulating material exhibiting a concave profile. In still another more detailed embodiment, the at least three electrodes of the first jaw are equidistantly spaced radially across the longitudinal cross-section thereof, and the at least three electrodes of the second jaw are equidistantly spaced radially across the longitudinal cross-section thereof.

In a more detailed embodiment of the first aspect, each of the at least three electrodes of the first jaw is configured to overlap another of each of the at least three electrodes of the second jaw. In yet another more detailed embodiment, at least one of the first jaw and the second jaw includes a vacuum port configured to be in fluid communication with a vacuum source. In a further detailed embodiment, the second jaw includes the vacuum port configured to be in fluid communication with the vacuum source, the vacuum port includes a plurality of vacuum ports, and the plurality of vacuum ports interpose the at least three electrodes. In still a further detailed embodiment, the longitudinal cross-section of the first jaw includes the insulating material exhibiting an arcuate concave profile, and the longitudinal cross-section of the second jaw includes the insulating material exhibiting an arcuate concave profile. In a more detailed embodiment, a radius of curvature of the arcuate concave profile of the insulating material of the first jaw is less than a radius of curvature of the arcuate concave profile of the insulating material of the second jaw. In a more detailed embodiment, a radius of curvature of the arcuate concave profile of the insulating material of the first jaw is equal to a radius of curvature of the arcuate concave profile of the insulating material of the second jaw. In another more detailed embodiment, a radius of curvature of the arcuate concave profile of the insulating material of the first jaw is greater than a radius of curvature of the arcuate concave profile of the insulating material of the second jaw.

It is a second aspect of the present invention to provide a method of performing a tissue ablation, the method comprising: (i) interposing tissue between a first bank of at least three electrodes and a second bank of at least three electrodes, where the first bank of at least three electrodes includes a first electrode, a second electrode, and a third electrode at least partially surrounded by an insulating material, and where the second bank of at least three electrodes includes a first electrode, a second electrode, and a third electrode at least partially surrounded by an insulating material, wherein the first electrode of the first bank is vertically aligned with the first electrode of the second bank, and the second electrode of the first bank is vertically offset from the first electrode of the first bank and is vertically aligned with the second electrode of the second bank, and the third electrode of the first bank is vertically offset from the first and second electrodes of the first bank and is vertically aligned with the third electrode of the second bank; (ii) determining a straight line distance between the first bank and the second bank after tissue interposes the first and second banks; and, (iii) delivering pulse field ablation energy to the tissue through at least one of the at least three electrodes of the first and second banks to the tissue interposing the first bank and the second bank.

In a more detailed embodiment of the second aspect, for a first straight line distance, delivering pulse field ablation energy to the tissue as follows: (a) delivering pulse field ablation energy to the tissue across the first electrode of the first bank to the second electrode of the second bank; (b) delivering pulse field ablation energy to the tissue across the third electrode of the first bank to the second electrode of the second bank; and, (c) delivering pulse field ablation energy to the tissue across the second electrode of the first bank to the second electrode of the second bank. In yet another more detailed embodiment, for the first straight line distance, delivering pulse field ablation energy as follows: (a) delivering pulse field ablation energy to the tissue across the second electrode of the first bank to the first electrode of the second bank; and (b) delivering pulse field ablation energy to the tissue across the second electrode of the first bank to the third electrode of the second bank. In a further detailed embodiment, for a second straight line distance, delivering pulse field ablation energy to the tissue as follows: (a) delivering pulse field ablation energy to the tissue across the first electrode of the first bank to the first electrode of the second bank; (b) delivering pulse field ablation energy to the tissue across the second electrode of the first bank to the second electrode of the second bank; and, (c) delivering pulse field ablation energy to the tissue across the third electrode of the first bank to the third electrode of the second bank. In still a further detailed embodiment, for the second straight line distance, delivering pulse field ablation energy to the tissue as follows: (a) delivering pulse field ablation energy to the tissue across the first electrode of the first bank to the third electrode of the second bank; and (b) delivering pulse field ablation energy to the tissue across the third electrode of the first bank to the first electrode of the second bank. In a more detailed embodiment, for a third straight line distance, delivering pulse field ablation energy to the tissue as follows: (a) delivering pulse field ablation energy to the tissue across the first electrode of the first bank to the first electrode of the second bank; and, (b) delivering pulse field ablation energy to the tissue across the third electrode of the first bank to the third electrode of the second bank. In a more detailed embodiment, for the third straight line distance, delivering pulse field ablation energy to the tissue as follows: (a) delivering pulse field ablation energy to the tissue across the first electrode of the first bank to the third electrode of the second bank; and, (b) delivering pulse field ablation energy to the tissue across the third electrode of the first bank to the first electrode of the second bank.

It is a third aspect of the present invention to provide a method of performing a tissue ablation, the method comprising: (i) positioning tissue adjacent a first bank of at least three electrodes, where the first bank of at least three electrodes includes a first electrode, a second electrode, and a third electrode at least partially surrounded by an insulating material, and where a longitudinal cross-section of the insulating material exhibits a concave profile and the at least three electrodes are distributed along the concave profile spaced apart from one another; (ii) determining a thickness of tissue adjacent to the first bank; and, (iii) delivering pulse field ablation energy to the tissue through at least one of the at least three electrodes of the first bank to the tissue adjacent to the first bank.

In a more detailed embodiment of the third aspect, the second electrode is spaced from the first electrode a first distance, the third electrode is spaced from the first electrode a second distance, greater than the first distance, and delivering the pulse field ablation energy to the tissue across the first electrode to the third electrode. In yet another more detailed embodiment, the method further includes applying vacuum to the tissue using a vacuum port in the insulating material as the pulse field ablation energy is delivered to the tissue. In a further detailed embodiment, the method further includes further comprising applying vacuum to the tissue using a vacuum port in the insulating material as the pulse field ablation energy is delivered to the tissue, where: (a) the second electrode is spaced from the first electrode a first distance; (b) the third electrode is spaced from the first electrode a second distance, greater than the first distance; (c) the vacuum port includes a plurality of vacuum ports; (d) a first of the plurality of vacuum ports interposes the first and second electrodes; and, (e) a second of the plurality of vacuum ports interposes the second and third electrodes. In still a further detailed embodiment, the method further includes compressing the tissue between a second bank of at least three electrodes and the first bank, where the second bank of at least three electrodes includes a first electrode, a second electrode, and a third electrode at least partially surrounded by an insulating material. In a more detailed embodiment, the second electrode of each of the first and second banks is spaced from the first electrode of each of the first and second banks a first distance, the third electrode of each of the first and second banks is spaced from the first electrode of each of the first and second banks a second distance, greater than the first distance, and delivering the pulse field ablation energy to the tissue across: (a) the first electrode of the first bank to the third electrode of the first bank; and, (b) the first electrode of the second bank to the third electrode of the second bank. In a more detailed embodiment, the method further includes applying vacuum to the tissue using a vacuum port in the insulating material of at least one of the first and second banks as the pulse field ablation energy is delivered to the tissue. In another more detailed embodiment, the method further includes applying vacuum to the tissue using a vacuum port in the insulating material of both of the first and second banks as the pulse field ablation energy is delivered to the tissue.

It is a fourth aspect of the present invention to provide a surgical device, comprising: (i) a handle; (ii) a shaft coupled to the handle; and (iii) a first jaw and a second jaw operatively coupled to a distal end of the shaft, where the first jaw is repositionable with respect to the second jaw, where each of the first jaw and the second jaw comprises an electrode extending beyond an insulating material, where each of the first jaw and the second jaw terminates at a respective tip, and where a potentiometer is operatively coupled to the first jaw and is configured to vary an output voltage as a function of a position of the first jaw with respect to the second jaw.

In a more detailed embodiment of the fourth aspect, the potentiometer is configured to be in electrical communication with a pulse frequency ablation controller. In yet another more detailed embodiment, a first component of the potentiometer is fixedly mounted to the handle, and a second component of the potentiometer is fixedly mounted to a repositionable linkage within the handle, the repositionable linkage operatively coupled to the first jaw. In a further detailed embodiment, the first component of the potentiometer comprises dual terminals of a resistive element, and the second component of the potentiometer comprises at least one of a pogo pin, a cantilever finger, and a spring arm. In still a further detailed embodiment, the first component of the potentiometer comprises at least one of a pogo pin, a cantilever finger, and a spring arm, and the second component of the potentiometer comprises dual terminals of a resistive element. In a more detailed embodiment, the handle houses an actuator mechanism that includes a return spring biasing a housing operatively coupled to the repositionable linkage. In a more detailed embodiment, the actuator mechanism further includes a force limiting spring and a relief rod operatively coupled to the housing. In another more detailed embodiment, the relief rod is operatively coupled to a plunger repositionably mounted to the handle. In yet another more detailed embodiment, the potentiometer comprises a linear potentiometer. In still another more detailed embodiment, the potentiometer comprises a sealed linear membrane potentiometer.

It is a fifth aspect of the present invention to provide a surgical device, comprising: (i) a handle; (ii) a shaft coupled to the handle; and (iii) a first jaw and a second jaw operatively coupled to a distal end of the shaft, where the first jaw is repositionable with respect to the second jaw, where each of the first jaw and the second jaw comprises an electrode extending beyond an insulating material, where each of the first jaw and the second jaw terminates at a respective tip, and where a Hall effect sensor is operatively coupled to the first jaw and is configured to vary an output voltage as a function of a position of the first jaw with respect to the second jaw.

In yet another more detailed embodiment of the firth aspect, the Hall effect sensor is configured to be in electrical communication with a pulse frequency ablation controller. In yet another more detailed embodiment, the Hall effect sensor is fixedly mounted to the handle, and a magnet is fixedly mounted to a repositionable linkage within the handle, the repositionable linkage operatively coupled to the first jaw. In a further detailed embodiment, a magnet is fixedly mounted to the handle, and the Hall effect sensor is fixedly mounted to a repositionable linkage within the handle, the repositionable linkage operatively coupled to the first jaw. In still a further detailed embodiment, the magnet is at least one of a permanent magnet and an electromagnet. In a more detailed embodiment, the handle houses an actuator mechanism that includes a return spring biasing a housing operatively coupled to the repositionable linkage. In a more detailed embodiment, the actuator mechanism further includes a force limiting spring and a relief rod operatively coupled to the housing. In another more detailed embodiment, the relief rod is operatively coupled to a plunger repositionably mounted to the handle.

It is a sixth aspect of the present invention to provide a surgical device, comprising: (i) a handle; (ii) a shaft coupled to the handle; and (iii) a first jaw and a second jaw operatively coupled to a distal end of the shaft, where the first jaw is repositionable with respect to the second jaw, where each of the first jaw and the second jaw comprises an electrode extending beyond an insulating material, where each of the first jaw and the second jaw terminates at a respective tip, and where a plurality of continuity switches are operatively coupled to the first jaw and are configured to vary an output voltage as a function of a position of the first jaw with respect to the second jaw.

In a more detailed embodiment of the sixth aspect, the plurality of continuity switches are configured to be in electrical communication with a pulse frequency ablation controller. In yet another more detailed embodiment, a fixture configured to close at least one of the continuity switches is fixedly mounted to the handle, and at least one of the plurality of continuity switches is fixedly mounted to a repositionable linkage within the handle, the repositionable linkage operatively coupled to the first jaw. In a further detailed embodiment, at least one of the plurality of continuity switches is fixedly mounted to the handle, and a fixture configured to close at least one of the continuity switches is fixedly mounted to a repositionable linkage within the handle, the repositionable linkage operatively coupled to the first jaw. In still a further detailed embodiment, a plurality of fixtures, configured to close multiples of the plurality of continuity switches, is fixedly mounted to the handle, and the plurality of continuity switches are fixedly mounted to a repositionable linkage within the handle, the repositionable linkage operatively coupled to the first jaw. In a more detailed embodiment, the plurality of continuity switches are fixedly mounted to the handle, and a plurality of fixtures, configured to close multiples of the plurality of continuity switches, are fixedly mounted to a repositionable linkage within the handle, the repositionable linkage operatively coupled to the first jaw. In a more detailed embodiment, the handle houses an actuator mechanism that includes a return spring biasing a housing operatively coupled to the repositionable linkage. In another more detailed embodiment, the actuator mechanism further includes a force limiting spring and a relief rod operatively coupled to the housing. In yet another more detailed embodiment, the relief rod is operatively coupled to a plunger repositionably mounted to the handle and/or each of the plurality of continuity switches includes it own resistor having a unique resistance.

It is a seventh aspect of the present invention to provide a method of calibrating a surgical device, the method comprising: (i) operating a surgical device to clamp a first test article having a first known width between a first jaw and a second jaw of the surgical device, wherein a first output signal is generated during clamping of the first test article; (ii) operating the surgical device to clamp a second test article having a second known width, different than the first known width, between the first jaw and the second jaw of the surgical device, wherein a second output signal is generated during clamping of the second test article; (iii) determine a transfer function describing the first and second output signals as a function of the first and second known widths; (iv) operating the surgical device to clamp a third test article having a third known width, different than the first and second known widths, between the first jaw and the second jaw of the surgical device, wherein a third output signal is generated during clamping of the third test article; and, (v) comparing a predicted width of the third test article using the transfer function against the third known width to discern whether the predicted width is within acceptable tolerances of the third known width.

In yet another more detailed embodiment of the seventh aspect, the surgical device includes a Hall effect sensor operatively coupled to at least one of the first and second jaws, the Hall effect sensor generates the first, second, and third output signals, and the first, second, and third output signals comprise differing voltages that vary responsive to the spacing between the first and second jaws. In yet another more detailed embodiment, the surgical device includes a potentiometer operatively coupled to at least one of the first and second jaws, the potentiometer generates the first, second, and third output signals, and the first, second, and third output signals comprise differing voltages that vary responsive to the spacing between the first and second jaws. In a further detailed embodiment, the surgical device includes a plurality of continuity switches operatively coupled to at least one of the first and second jaws, the plurality of continuity switches contribute to the generation of the first, second, and third output signals, and the first, second, and third output signals comprise differing voltages that vary responsive to the spacing between the first and second jaws. In still a further detailed embodiment, the first test article comprises a first test cylinder having a first known diameter, the second test article comprises a second test cylinder having a second known diameter, the third test article comprises a third test cylinder having a third known diameter, and comparing the predicted width of the third test article using the transfer function against the third known width to discern whether the predicted width is within acceptable tolerances of the third known width includes comparing a predicted diameter of the third test cylinder using the transfer function against the third known diameter to discern whether the predicted diameter is within acceptable tolerances of the third known diameter. In a more detailed embodiment, the method further includes storing the transfer function in a memory associated with the surgical device, wherein the memory is configured to be accessible by a surgical device controller.

It is an eighth aspect of the present invention to provide a method of operating a pulse field ablation surgical device having a pair of ablation jaws, the method comprising: (i) establishing communication between the pulse field ablation surgical device and a surgical device controller, where establishing communication includes the surgical device controller accessing from memory of the pulse field ablation surgical device a transfer function correlating signal outputs to spacing between the pair of ablation jaws; (ii) clamping tissue between the pair of ablation jaws and generating a signal output indicative of the spacing between the pair of ablation jaws while the tissue is clamped; (iii) using the transfer function to calculate the distance between the pair of ablation jaws using the signal output; and, (iv) delivering pulse field ablation energy via the surgical device controller using the calculated distance between the pair of ablation jaws.

In yet another more detailed embodiment of the eighth aspect, the method further includes using at least one of a Hall effect sensor, continuity switches, and a potentiometer to generate the signal output, and based upon the calculated distance, assigning one of a plurality of zones to the clamped tissue, where each of the plurality of zones is assigned its own unique pulse field ablation energy. In yet another more detailed embodiment, the pair of ablation jaws includes a first jaw and a second jaw, each of the first jaw and the second jaw includes a first electrode, a second electrode, and a third electrode laterally distributed across an insulating material and spaced apart from one another, the first electrode, the second electrode, and the third electrode of the first jaw are configured to respectively overlap the first electrode, the second electrode, and the third electrode of the second jaw when tissue is clamped therebetween, and delivering the pulse field ablation energy includes delivering the pulse field ablation energy across respective first electrodes, second electrodes, and third electrodes in parallel. In a further detailed embodiment, the pair of ablation jaws includes a first jaw and a second jaw, each of the first jaw and the second jaw includes a first electrode, a second electrode, and a third electrode laterally distributed across an insulating material and spaced apart from one another, the first electrode, the second electrode, and the third electrode of the first jaw are configured to respectively overlap the first electrode, the second electrode, and the third electrode of the second jaw when tissue is clamped therebetween, and delivering the pulse field ablation energy includes delivering the pulse field ablation energy across at least one of: (i) the first electrode of the first jaw and the second or third electrode of the second jaw, (ii) the second electrode of the first jaw and the first or third electrode of the second jaw, (iii) the third electrode of the first jaw and the first or second electrode of the second jaw. In still a further detailed embodiment, the method further includes applying suction to the tissue via at least one of the pair of ablation jaws.

It is a ninth aspect of the present invention to provide a surgical device, comprising a first bank of electrodes repositionable with respect to a second bank of electrodes, where each of the first bank of electrodes and the second bank of electrodes comprises at least three electrodes exposed with respect to an insulating material and spaced radially across a longitudinal cross-section thereof.

In yet another more detailed embodiment of the ninth aspect, the longitudinal cross-section of the first bank of electrodes includes the insulating material exhibiting a convex profile, and the longitudinal cross-section of the second bank of electrodes includes the insulating material exhibiting a convex profile. In yet another more detailed embodiment, the at least three electrodes of the first bank of electrodes are equidistantly spaced radially across the longitudinal cross-section thereof, and the at least three electrodes of the second bank of electrodes are equidistantly spaced radially across the longitudinal cross-section thereof. In a further detailed embodiment, each of the at least three electrodes of the first bank of electrodes is configured to overlap another of each of the at least three electrodes of the second bank of electrodes. In still a further detailed embodiment, the longitudinal cross-section of the first bank of electrodes includes the insulating material exhibiting a convex arcuate profile, and the longitudinal cross-section of the second bank of electrodes includes the insulating material exhibiting a convex arcuate profile. In a more detailed embodiment, at least one or two of the at least three electrodes extends from an apex of the convex arcuate profile of the first bank of electrodes, and at least one or two of the at least three electrodes extends from an apex of the convex arcuate profile of the second bank of electrodes. In a more detailed embodiment, at least one of the at least three electrodes is recessed with respect to an apex of the convex arcuate profile of the first bank of electrodes, and at least one of the at least three electrodes is recessed with respect to an apex of the convex arcuate profile of the second bank of electrodes. In another more detailed embodiment, the longitudinal cross-section of the first bank of electrodes includes the insulating material exhibiting a convex profile, and the longitudinal cross-section of the second bank of electrodes includes the insulating material exhibiting a concave profile. In yet another more detailed embodiment, the at least three electrodes of the first bank of electrodes are equidistantly spaced radially across the longitudinal cross-section thereof, and the at least three electrodes of the second bank of electrodes are equidistantly spaced radially across the longitudinal cross-section thereof. In still another more detailed embodiment, each of the at least three electrodes of the first bank of electrodes is configured to overlap another of each of the at least three electrodes of the second bank of electrodes.

In a more detailed embodiment of the ninth aspect, at least one of the first bank of electrodes and the second bank of electrodes includes a vacuum port configured to be in fluid communication with a vacuum source. In yet another more detailed embodiment, the second bank of electrodes includes the vacuum port configured to be in fluid communication with the vacuum source, the vacuum port includes a plurality of vacuum ports, and the plurality of vacuum ports interpose the at least three electrodes. In a further detailed embodiment, the longitudinal cross-section of the first bank of electrodes includes the insulating material exhibiting an arcuate convex profile, and the longitudinal cross-section of the second bank of electrodes includes the insulating material exhibiting an arcuate concave profile. In still a further detailed embodiment, a radius of curvature of the arcuate convex profile is less than a radius of curvature of the arcuate concave profile. In a more detailed embodiment, a radius of curvature of the arcuate convex profile is equal to a radius of curvature of the arcuate concave profile. In a more detailed embodiment, a radius of curvature of the arcuate convex profile is greater than a radius of curvature of the arcuate concave profile. In another more detailed embodiment, the longitudinal cross-section of the second bank of electrodes includes the insulating material exhibiting a concave arcuate profile with opposed lateral rims, and the at least three electrodes are recessed below a height of the opposed lateral rims.

In yet another more detailed embodiment of the ninth aspect, the longitudinal cross-section of the first bank of electrodes includes the insulating material exhibiting a concave profile, and the longitudinal cross-section of the second bank of electrodes includes the insulating material exhibiting a concave profile. In yet another more detailed embodiment, the at least three electrodes of the first bank of electrodes are equidistantly spaced radially across the longitudinal cross-section thereof, and the at least three electrodes of the second bank of electrodes are equidistantly spaced radially across the longitudinal cross-section thereof. In a further detailed embodiment, each of the at least three electrodes of the first bank of electrodes is configured to overlap another of each of the at least three electrodes of the second bank of electrodes. In still a further detailed embodiment, at least one of the first bank of electrodes and the second bank of electrodes includes a vacuum port configured to be in fluid communication with a vacuum source. In a more detailed embodiment, the second bank of electrodes includes the vacuum port configured to be in fluid communication with the vacuum source, the vacuum port includes a plurality of vacuum ports, and the plurality of vacuum ports interpose the at least three electrodes. In a more detailed embodiment, the longitudinal cross-section of the first bank of electrodes includes the insulating material exhibiting an arcuate concave profile, and the longitudinal cross-section of the second bank of electrodes includes the insulating material exhibiting an arcuate concave profile. In another more detailed embodiment, a radius of curvature of the arcuate concave profile of the insulating material of the first bank of electrodes is less than a radius of curvature of the arcuate concave profile of the insulating material of the second bank of electrodes. In yet another more detailed embodiment, a radius of curvature of the arcuate concave profile of the insulating material of the first bank of electrodes is equal to a radius of curvature of the arcuate concave profile of the insulating material of the second bank of electrodes. In still another more detailed embodiment, a radius of curvature of the arcuate concave profile of the insulating material of the first bank of electrodes is greater than a radius of curvature of the arcuate concave profile of the insulating material of the second bank of electrodes.

### BRIEF DESCRIPTION OF THE DRAWINGS

The description of the illustrative embodiments can be read in conjunction with the accompanying figures. It will be appreciated that for simplicity and clarity of illustration, elements illustrated in the figures have not necessarily been drawn to scale. For example, the dimensions of some of the elements may be exaggerated relative to other elements. Embodiments incorporating teachings of the present disclosure are shown and described with respect to the figures presented herein.

FIG. 1 is a simplified schematic diagram of an example PFA system 100, according to at least some aspects of the present disclosure.

FIG. 2 is a perspective view of an example clamp-type PFA device 200, according to at least some aspects of the present disclosure.

FIG. 3A is a perspective view of an example minimally invasive PFA device 300, according to at least some aspects of the present disclosure.

FIG. 3B is a simplified schematic view of the PFA device 300 of FIG. 3A.

FIG. 4A is a perspective view of an example needle-type PFA device 400, according to at least some aspects of the present disclosure.

FIG. 4B is a perspective view of the example needle-type PFA device 400 of FIG. 4A.

FIG. 4C is a magnified perspective view of a distal end of the example needle-type PFA device 400 of FIG. 4A.

FIG. 5A illustrates an elongated, generally linear electrode, according to at least some aspects of the present disclosure.

FIG. 5B illustrates a point electrode, according to at least some aspects of the present disclosure.

FIG. 5C illustrates a segmented electrode, according to at least some aspects of the present disclosure.

FIG. 5D illustrates an example nested electrode arrangement, according to at least some aspects of the present disclosure.

FIG. 5E illustrates an example squiggle electrode, according to at least some aspects of the present disclosure.

FIG. 5F illustrates an example perpendicular electrode arrangement, according to at least some aspects of the present disclosure.

FIG. 5G illustrates an example parallel electrode arrangement, according to at least some aspects of the present disclosure.

FIG. 5H illustrates an example continuous electrode, according to at least some aspects of the present disclosure.

FIG. 5I illustrates two example electrode arrays, according to at least some aspects of the present disclosure.

FIG. 5J illustrates example plate electrodes, according to at least some aspects of the present disclosure.

FIG. 5K illustrates an example electrode configuration including multiple pairs of cooperating electrodes, according to at least some aspects of the present disclosure.

FIG. 5L illustrates example raised electrodes, according to at least some aspects of the present disclosure.

FIG. 5M illustrates an example electrode configuration including raised electrodes positioned opposite a plate electrode, according to at least some aspects of the present disclosure.

FIG. 5N is a simplified section view of an alternative example electrode configuration that may be used, for example, in connection with PFA devices generally similar to PFA devices shown in FIGS. 2, 3A, 4A, and 22, according to at least some aspects of the present disclosure.

FIG. 5O is a simplified section view of an alternative example electrode configuration that may be used, for example, in connection with PFA devices generally similar to PFA devices shown in FIGS. 2, 3A, 4A, and 22, according to at least some aspects of the present disclosure.

FIG. 5P is a simplified section view of an alternative example electrode configuration that may be used, for example, in connection with PFA devices generally similar to PFA devices shown in FIGS. 2, 3A, 4A, and 22, according to at least some aspects of the present disclosure.

FIG. 5Q is a simplified section view of an alternative example electrode configuration that may be used, for example, in connection with PFA devices generally similar to PFA devices shown in FIGS. 2, 3A, 4A, and 22, according to at least some aspects of the present disclosure.

FIG. 5R is a simplified section view of an alternative example electrode configuration that may be used, for example, in connection with PFA devices generally similar to PFA devices shown in FIGS. 2, 3A, 4A, and 22, according to at least some aspects of the present disclosure.

FIG. 5S is a simplified section view of an alternative example electrode configuration that may be used, for example, in connection with PFA devices generally similar to PFA devices shown in FIGS. 2, 3A, 4A, and 22, according to at least some aspects of the present disclosure.

FIG. 5T is a simplified section view of an alternative example electrode configuration that may be used, for example, in connection with PFA devices generally similar to PFA devices shown in FIGS. 2, 3A, 4A, and 22, according to at least some aspects of the present disclosure.

FIG. 6A is a simplified section view of a vacuum-clamp configuration, according to at least some aspects of the present disclosure.

FIG. 6B illustrates a baseline example embodiment including substantially flat opposed tissue engagement surfaces, according to at least some aspects of the present disclosure.

FIG. 6C illustrates an example embodiment including opposed convex tissue engagement surfaces, according to at least some aspects of the present disclosure.

FIG. 6D illustrates an example embodiment including opposed concave tissue engagement surfaces, according to at least some aspects of the present disclosure.

FIG. 6E illustrates an example embodiment including a convex tissue engagement surface opposing a cooperating concave tissue engagement surface, according to at least some aspects of the present disclosure.

FIG. 7 is a graphical illustration of the composition of an example multiple burst PFA signal, according to at least some aspects of the present disclosure.

FIG. 8 is a table listing example PFA signal parameters that may be used in connection with a variety of PFA devices, according to at least some aspects of the present disclosure.

FIG. 9 is a cross-sectional view showing electrode placing with respect to one another and on opposites of tissue as part of explaining bipolar/monopolar configurations and biphasic/monophasic signals, according to at least some aspects of the present disclosure.

FIG. 10A is a plot of an example ECG trace, according to at least some aspects of the present disclosure.

FIG. 10B illustrates two example embodiments configured to mechanically measure the distance between opposed jaws, according to at least some aspects of the present disclosure.

FIG. 10C illustrates four example embodiments configured to electrically and/or electronically measure the distance between opposed jaws, according to at least some aspects of the present disclosure.

FIG. 10D illustrates an example ratcheting clamp mechanism, according to at least some aspects of the present disclosure.

FIG. 11A is a perspective view of an example insulator configuration comprising a compressible insulator at least partially circumscribing one or more electrodes, according to at least some aspects of the present disclosure.

FIG. 11B is a different perspective view of the example insulator configuration depicted in FIG. 11A.

FIG. 11C is a simplified section view of the embodiment of FIG. 11A.

FIG. 12A is a perspective view of an example insulator configuration forming a shortened electrode exposure region, according to at least some aspects of the present disclosure.

FIG. 12B is a simplified section view of the embodiment of FIG. 12A.

FIG. 12C is another simplified section view of the embodiment of FIG. 12A.

FIG. 13A is a perspective view of an example configuration comprising an insulated jaw, according to at least some aspects of the present disclosure.

FIG. 13B is a section view of the embodiment of FIG. 13A.

FIG. 13C is a simplified perspective view of an alternative embodiment in which an electrode is selectively insulated, according to at least some aspects of the present disclosure.

FIG. 14A is a perspective view of an example configuration comprising a jaw with selectively insulated electrodes, according to at least some aspects of the present disclosure.

FIG. 14B is a section view of the embodiment of FIG. 14A.

FIG. 14C is another section view of the embodiment of FIG. 14A.

FIG. 15A is a perspective view of an alternative example configuration comprising a jaw with selectively insulated electrodes, according to at least some aspects of the present disclosure.

FIG. 15B is a section view of the embodiment of FIG. 15A.

FIG. 15C is another section view of the embodiment of FIG. 15A.

FIG. 16A is a perspective view of an alternative example configuration comprising a jaw with selectively insulated electrodes, according to at least some aspects of the present disclosure.

FIG. 16B is a section view of the embodiment of FIG. 16A.

FIG. 16C is another section view of the embodiment of FIG. 16A.

FIG. 17A is a perspective view of an alternative example configuration comprising a jaw with selectively insulated electrodes, according to at least some aspects of the present disclosure.

FIG. 17B is a section view of the embodiment of FIG. 17A.

FIG. 17C is another section view of the embodiment of FIG. 17A.

FIG. 18A is a perspective view of an alternative example configuration comprising a jaw with selectively insulated electrodes, according to at least some aspects of the present disclosure.

FIG. 18B is a section view of the embodiment of FIG. 18A.

FIG. 18C is another section view of the embodiment of FIG. 18A.

FIG. 18D illustrates an example embodiment including a deformable insulator disposed about an electrode disposed on a rigid back plate, according to at least some aspects of the present disclosure.

FIG. 18E illustrates an example embodiment including spaced-apart posts supporting an electrode within a deformable insulator, according to at least some aspects of the present disclosure.

FIG. 18F illustrates an example embodiment including raised features of a deformable insulator configured to contact tissue and expose an electrode, according to at least some aspects of the present disclosure.

FIG. 18G illustrates an alternative example embodiment in which a deformable insulator may be segmented, according to at least some aspects of the present disclosure.

FIG. 19A is a top view of an example lesion including a PFA zone and a thermal ablation zone, according to at least some aspects of the present disclosure.

FIG. 19B is a section view of the lesion of FIG. 19A.

FIG. 19C is a top view of an example lesion created using a PFA and RF device, depicting a PFA zone and a thermal ablation zone, according to at least some aspects of the present disclosure.

FIG. 19D illustrates an example snare clamp, according to at least some aspects of the present disclosure.

FIG. 19E illustrates an example generally helical screw engagement element for a jaw or electrode configured to penetrate the target tissue, according to at least some aspects of the present disclosure.

FIG. 20 is a simplified lateral view of an example PFA device including an expandable structure, according to at least some aspects of the present disclosure.

FIG. 21 is a simplified block diagram of an example equipment configuration, which may be used, for example, for using various PFA and/or RF ablation devices and/or algorithms according to at least some aspects of the present disclosure.

FIG. 22 is a perspective view of an example minimally invasive PFA device, according to at least some aspects of the present disclosure.

FIG. 23 is a table listing exemplary parameters or settings for operating a PFA device, according to at least some aspects of the present disclosure.

FIG. 24A is a longitudinal, cross-sectional view of an exemplary insulator and electrode configuration for an exemplary PFA clamp where each insulator is convex.

FIG. 24B is a longitudinal, cross-sectional view of the exemplary insulator and electrode configuration for an exemplary PFA clamp of FIG. 24A showing generation of elongated energy fields between non-overlapping electrodes.

FIG. 24C is a longitudinal, cross-sectional view of the exemplary insulator and electrode configuration for an exemplary PFA clamp of FIG. 24A showing generation of an elongated energy fields between non-overlapping electrodes.

FIG. 24D is a longitudinal, cross-sectional view of the exemplary insulator and electrode configuration for an exemplary PFA clamp of FIG. 24A showing generation of an elongated energy fields between overlapping electrodes.

FIG. 24E are longitudinal, cross-sectional views of the exemplary insulator and electrode configuration for an exemplary PFA clamp of FIG. 24A showing generation of elongated energy fields between overlapping and non-overlapping electrodes at a first tissue spacing.

FIG. 24F are longitudinal, cross-sectional views of the exemplary insulator and electrode configuration for an exemplary PFA clamp of FIG. 24A showing generation of elongated energy fields between overlapping and non-overlapping electrodes at a second tissue spacing.

FIG. 25A is a longitudinal, cross-sectional view of an exemplary insulator and electrode configuration for an exemplary PFA clamp where one insulator is convex and another insulator is concave.

FIG. 25B is a reproduction of the view of FIG. 25A with one or more embedded channels.

FIG. 25C is a longitudinal, cross-sectional views of the exemplary insulator and electrode configuration for an exemplary PFA clamp of FIG. 25A showing generation of elongated energy fields electrodes of the same jaw.

FIG. 26 is a longitudinal, cross-sectional view of an exemplary insulator and electrode configuration for an exemplary PFA clamp where both insulators are concave.

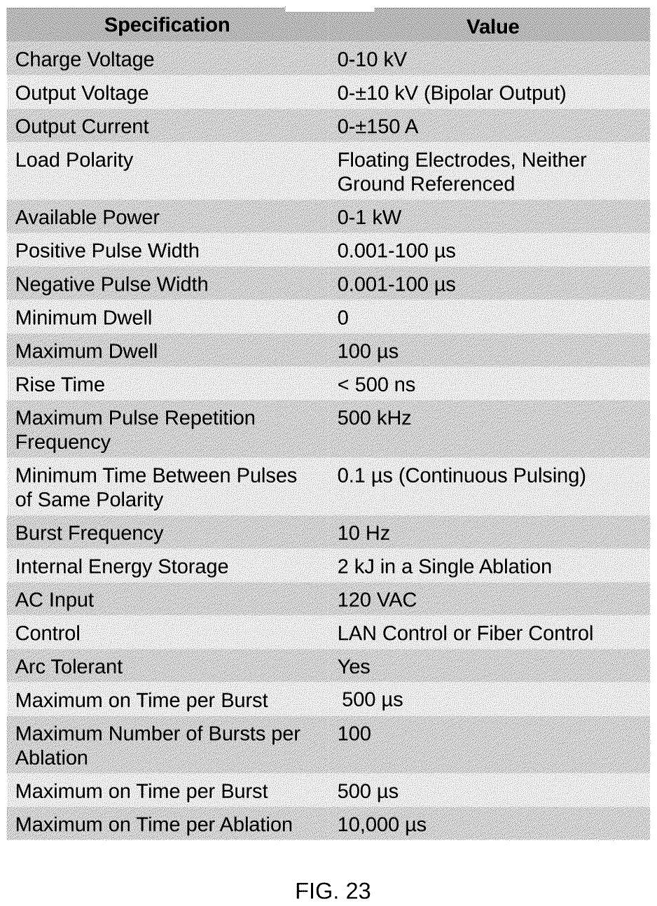

FIG. 23 is a table listing exemplary parameters or settings for operating a PFA device, according to at least some aspects of the present disclosure.

FIG. 27 is an elevated perspective view of an exemplary PFA device.

FIG. 28 is an exemplary circuit diagram for measuring impedance in order to determine jaw spacing of the exemplary PFA device of FIG. 27.

FIG. 29 is an exemplary circuit diagram for measuring voltage in order to determine jaw spacing of the exemplary PFA device of FIG. 27.

FIG. 30 is an elevated perspective view of a first exemplary portion of the exemplary PFA device of FIG. 27 with one of the handle housings removed.

FIG. 31 is a magnified view of some of the internal components depicted in FIG. 30.

FIG. 32 is an elevated perspective view of a second exemplary portion of the exemplary PFA device of FIG. 27 with one of the handle housings removed.

FIG. 33 is a profile view of the handle housing removed from FIG. 32.

FIG. 34 is a profile view of the second exemplary portion of the exemplary PFA device of FIGS. 27 and 32 with one of the handle housings removed.

FIG. 35 is a profile view of a third exemplary portion of the exemplary PFA device of FIG. 27 with one of the handle housings removed.

FIG. 36 is a profile view of a fourth exemplary portion of the exemplary PFA device of FIG. 27 with one of the handle housings removed.

FIG. 37 is a profile view of a fifth exemplary portion of the exemplary PFA device of FIG. 27 with one of the handle housings removed.

FIG. 38 is a profile view of a sixth exemplary portion of the exemplary PFA device of FIG. 27 with one of the handle housings removed.

FIG. 39 is a schematic diagram of an exemplary process for determining zones of ablation and the operating conditions for each zone.

FIG. 40 is a schematic diagram of an exemplary calibration sequence in accordance with the instant disclosure.

FIG. 41 is a schematic diagram of an exemplary sequence of carrying out a PFA ablation in accordance with the instant disclosure using the PFA device of FIG. 27.

FIG. 42 is a schematic diagram of a second exemplary sequence of carrying out a PFA ablation in accordance with the instant disclosure using the PFA device of FIG. 27.

FIG. 43 is a schematic diagram of a third exemplary sequence of carrying out a PFA ablation in accordance with the instant disclosure using the PFA device of FIG. 27.

FIG. 44 is a graphical depiction showing how output voltage varies as a function of magnetic flux density.

### DETAILED DESCRIPTION

Example embodiments according to the present disclosure are described and illustrated below to encompass devices, methods, and techniques relating to PFA. Of course, it will be apparent to those of ordinary skill in the art that the embodiments discussed below are examples and may be reconfigured without departing from the scope and spirit of the present disclosure. It is also to be understood that variations of the example embodiments contemplated by one of ordinary skill in the art shall concurrently comprise part of the instant disclosure. However, for clarity and precision, the example embodiments as discussed below may include optional steps, methods, and features that one of ordinary skill should recognize as not being a requisite to fall within the scope of the present disclosure. Unless explicitly stated otherwise, any feature or function described in connection with any example embodiment may apply to any other example embodiment, and repeated description of similar features and functions is omitted for brevity.

The present disclosure contemplates that PFA may kill cells by using high-intensity electrical fields to cause irreversible nanopore formation in cell membranes, which is known as irreversible electroporation (“IRE”). IRE may be used to create deep and uniform lesions in cardiac tissue, which can be useful for treating arrythmias. If the electrical signal applied to the target tissue has insufficient intensity to cause IRE, reversible electroporation may occur. Pores formed by reversible electroporation may not be permanent, and affected cells typically recover after a short period of time (e.g., hours, days, weeks). The present disclosure contemplates that the minimum electric field strength (or voltage) required to cause IRE may depend on the characteristics of the electrical signal that is applied to the target tissue and the target tissue itself. For example, the number of pulses, frequency, magnitude, duration, shape, etc., may affect the extent of electroporation. In some circumstances, a region of IRE may be at least partially bounded by a region of reversible electroporation.

For context, an example PFA protocol configured to cause IRE may include a series of energy pulses (i.e., 100 Volts direct current (VDC)) at a given duration (i.e., 100 microseconds (μs)) at a given frequency (i.e., 0.1 Hz to 10 Hz). For this sort of protocol, an electric field is applied to the tissue to create a cellular transmembrane voltage potential that may be in the range from 0.5 kV/cm to 8.0 kV/cm and optionally more preferably between 2 kV/cm to 7 kV/cm, depending on the tissue and how tissue damage is assessed. In some circumstances, electroporation efficacy may not be directly related to the amount of delivered energy or the charge. For example, in some circumstances, two 100 μs pulses of 1000 V/cm may be more effective for producing IRE than a single pulse of 200 μs with similar energy and charge. Additional details and alternatives are described elsewhere herein.

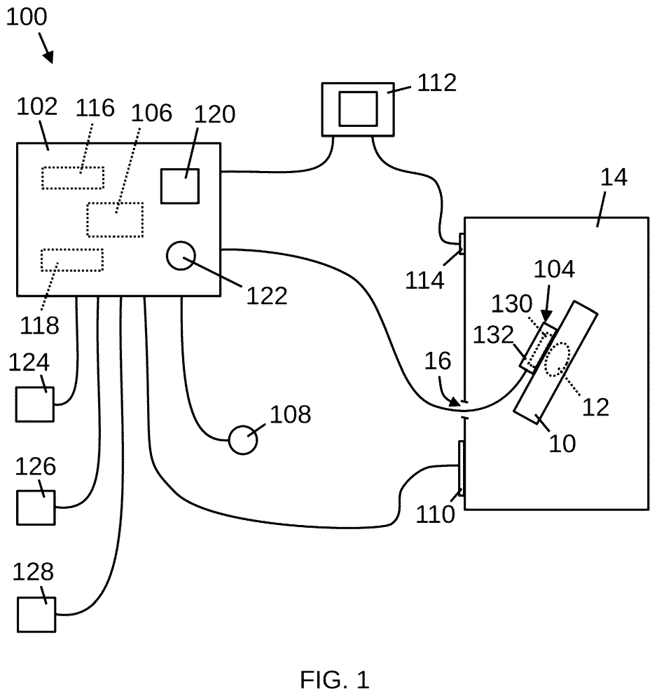

The following description of example embodiments with reference to FIGS. 1, 2, 3A, 3B, and 4A-4C provides context for various example apparatus features and methods described in more detail elsewhere herein. It is to be understood that any of these example embodiments may be utilized in connection with any feature or aspect described elsewhere herein.

Turning to FIG. 1, an example PFA system 100 may include a PFA unit 102, which may be operatively coupled to a PFA device 104. The PFA unit 102 may include a PFA generator 106, which may be configured to produce and/or supply electrical pulses for PFA. The PFA device 104 may be configured to apply the PFA pulses to a target tissue 10 in connection with creating a lesion 12 therein. In some example embodiments, the PFA unit 102 may be provided as capital (e.g., reusable) equipment and/or the PFA device 104 may be provided as disposable (e.g., single-use) equipment.

In some example embodiments, the target tissue 10 may be located internally within a patient's body 14. The PFA device 104 may be positioned proximate the target tissue 10 via any suitable patient access 16, such as arterial or venous access, percutaneous access, open surgical access, and/or minimally invasive surgical access. For example, in connection with treating cardiac arrhythmias, the target tissue 10 may include the heart wall (e.g., myocardium). In some example embodiments, the PFA device 104 may be positioned generally against the external (e.g., epicardial) surface of the heart wall and/or generally against the internal (e.g., endocardial) surface of the heart wall.

In some example embodiments, the PFA unit 102 may include and/or may be used in connection with various other components. For example, in some example embodiments, a foot switch 108 may be used to activate certain functions associated with the PFA unit 102, such as delivery of ablation energy to the PFA device 104. In some example embodiments, a return electrode 110 may be electrically coupled to the patient's body 14, such as to provide a return path for monopolar ablation energy delivered via the PFA device 104.

In some example embodiments, an electrocardiogram (“ECG”) monitor 112 may be used to display and/or analyze electrical impulses associated with the patient's heartbeat using one or more ECG electrodes 114. In some example embodiments, the ECG monitor 112 may be operatively coupled to and/or incorporated within the PFA unit 102, such as to facilitate synchronization of ablation pulse timing with the patient's heartbeat, as described below.

In some example embodiments, the PFA unit 102 may be configured to provide only PFA energy. In some example embodiments, the PFA unit 102 may be configured for use in connection with additional ablation modalities. For example, the PFA unit 102 may include and/or may be used in connection with one or more components configured for RF ablation, such as an RF generator 116, which may be generally similar to the “Ablation Sensing Unit (ASU),” “Ablation Switch Box (ASB),” and/or “Estech Electrosurgical unit (ESU)” available from AtriCure, Inc. of Mason, Ohio. As another example, the PFA unit 102 may include and/or may be used in connection with one or more components configured for cryosurgical ablation, such as a cryosurgical unit 118, which may be generally similar to the “cryoICE BOX” cryogenic surgical unit available from AtriCure, Inc. of Mason, Ohio. Generally, a particular lesion (or portion thereof) may be formed using PFA, one or more other ablation modalities, or any combination thereof, in any order sequentially and/or simultaneously (e.g., PFA and/or RF and/or cryo).

In some example embodiments, the PFA unit 102 may include one or more indicators and/or displays 120, which may provide information to the operator about the patient, the PFA unit 102, and/or an ablation. For example, some PFA units 102 may include integrated tissue interrogation/mapping functionality (such as voltage mapping, impedance mapping, exit/entrance block testing of lesions by cardiac pacing and sensing), which may use one or more dedicated electrodes and/or one or more electrodes associated with the PFA device 104. In some example embodiments, the PFA unit 102 may include one or more input devices 122, such as knobs, dials, switches, buttons, touch screens, etc., which may allow an operator to direct operation of various components of the PFA unit 102.

In some example embodiments, the PFA unit 102 may be configured with one or more external connections. For example, the PFA unit 102 may be operatively coupled to an electrical power source 124, such as a wall outlet. Some example embodiments may be operatively coupled to a vacuum source 126, such as an operating room vacuum system. Some example embodiments may be operatively coupled to a gas source 128, such as a compressed gas cylinder, which may contain a cryogenic fluid, for example.

In some example embodiments, the PFA device 104 may include one or more electrodes 130, which may be disposed in or on an end effector 132, to deliver PFA energy to the target tissue 10.

The description herein references a distal direction 18 and a proximal direction 20. The proximal direction 20 may be generally opposite the distal direction 18. As used herein, “distal” may refer to a direction generally away from an operator of a system or device (e.g., a surgeon), such as toward the distant-most end of a device that is inserted into a patient's body. As used herein, “proximal” may refer to a direction generally toward an operator of a system or device (e.g., a surgeon), such as away from the distant-most end of a device that is inserted into a patient's body. It is to be understood, however, that example directions referenced herein are merely for purposes of explanation and clarity, and should not be considered limiting.

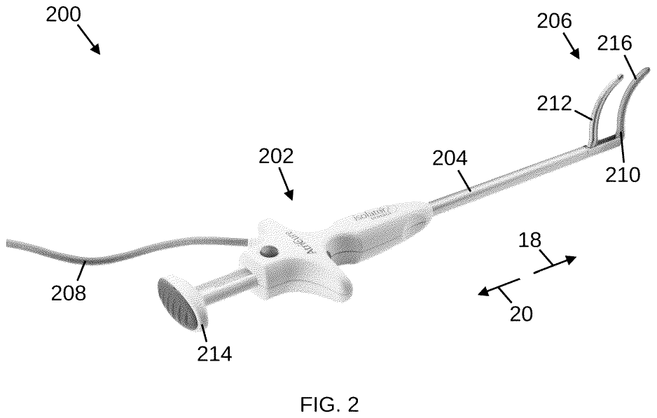

Referring to FIGS. 1 and 2, the illustrated clamp-type PFA device 200 may include a proximally disposed handle 202, a shaft 204 extending distally from the handle 202, and/or an end effector 206 disposed distally on the shaft 204. Generally, some example PFA devices 200 may be similar to the “Isolator Synergy” surgical ablation device available from AtriCure, Inc. of Mason, Ohio, and/or the devices described in U.S. Pat. No. 9,072,518, issued Jul. 7, 2015, titled “HIGH-VOLTAGE PULSE ABLATION SYSTEMS AND METHODS,” which is incorporated herein by reference in its entirety. Further, various PFA devices of any configuration and according to at least some aspects of the present disclosure may employ overlapping fields (e.g., focused between paired electrodes), on/off duty cycles (e.g., for thermal management), and/or constant signal generation switched to multiple electrode pairs generally similar to those utilized by the “Isolator Synergy” device and/or as described in U.S. Pat. No. 9,072,518.

Generally, any handle described herein with reference to any exemplary embodiment may be configured to be grasped by a human user (e.g., surgeon) and/or engaged by a non-human, mechanical and/or robotic device (e.g., a surgical robot). More generally, any handle described herein may comprise any structure that may be configured to be secured, held, and/or manipulated to position and/or restrain a PFA device, regardless of whether it may be held by a human (e.g., surgeon or assistant), robot, mechanical device, etc.

In the illustrated embodiment, a proximally extending connecting element 208 may electrically couple the PFA device 200 to the PFA unit 102. In some embodiments utilizing vacuum and/or cryogenics, the connecting element 208 may include suitable conduits. The end effector 206, corresponding to end effector 132, may comprise a distal repositionable or fixed jaw 210 and/or a movable proximal jaw 212. A plunger 214 or other actuator, which may be disposed proximally on the handle 202, may allow the operator to reposition one or both jaw 210, 212 to clamp the target tissue 10 between the jaws 210, 212. In the illustrated embodiment, one or both of the jaws 210, 212 may include one or more electrodes 216, corresponding to electrode 130, which may be utilized to deliver PFA energy to the target tissue 10. In embodiments including one or more electrodes, the electrode(s) may be positioned on either or both jaws 210, 212.

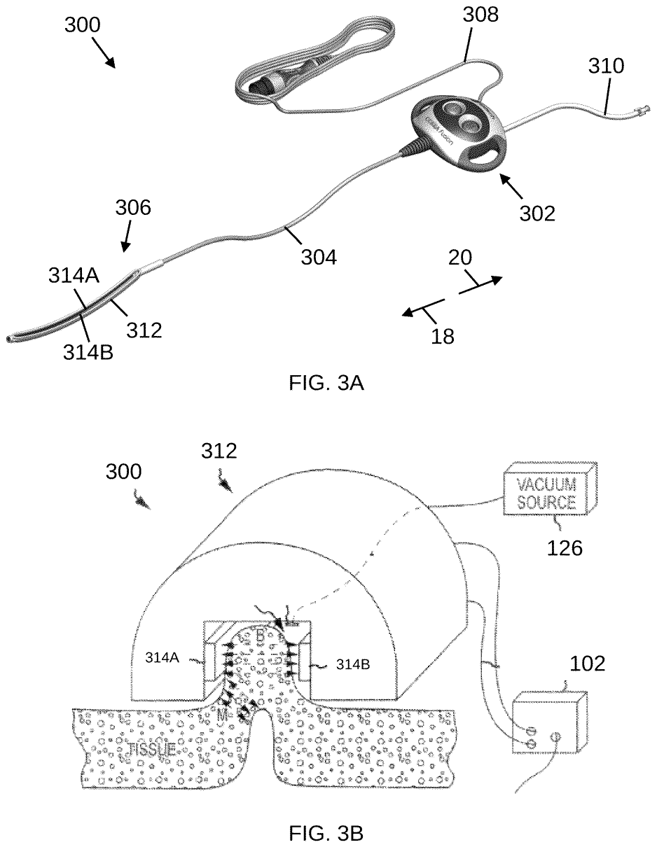

Referring to FIGS. 1, 3A, and 3B, the illustrated minimally invasive PFA device 300 may include a proximally disposed handle 302, a flexible connecting element 304 extending distally from the handle 302, and/or an end effector 306 disposed distally on the connecting element 304. Generally, some example PFA devices 300 may be similar to the “COBRA Fusion” ablation system available from AtriCure, Inc. of Mason, Ohio, and/or the devices described in U.S. Pat. No. 9,474,574, issued Oct. 25, 2016, titled “STABILIZED ABLATION SYSTEMS AND METHODS,” which is incorporated herein by reference in its entirety.

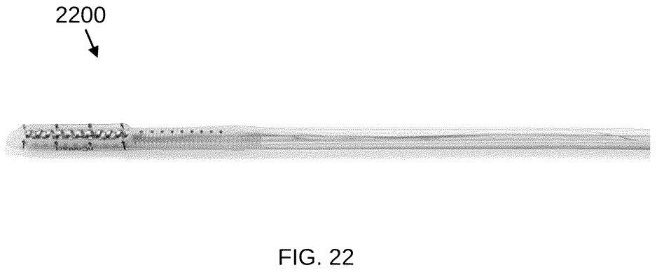

In the illustrated embodiment, a proximally extending electrical connecting element 308 may electrically couple the PFA device 300 to the PFA unit 102. A proximally extending vacuum connecting element 310 may fluidically couple the PFA device 300 to the PFA unit 102 and/or to the vacuum source 126. In the illustrated embodiment, the end effector 306, corresponding to end effector 132, may comprise an elongated, flexible stabilizer 312 configured to releasably engage the target tissue 10, such as by using vacuum. In the illustrated embodiment, one or more electrodes 314A, 314B, corresponding to electrode 130, may be disposed within the stabilizer 312 and/or may be utilized to deliver PFA energy to the target tissue 10. In some example embodiments, the PFA device 300 may be configured for vacuum-stabilized, unidirectional, and/or bipolar (or selectively bipolar/monopolar) operation. In some example embodiments, the target tissue may be folded, as generally shown in FIG. 3B. In some embodiments, the target tissue may be drawn into contact with the electrodes 314A, 314B without substantial tissue folding. The PFA device 2200 illustrated in FIG. 22 may be utilized in a similar manner.

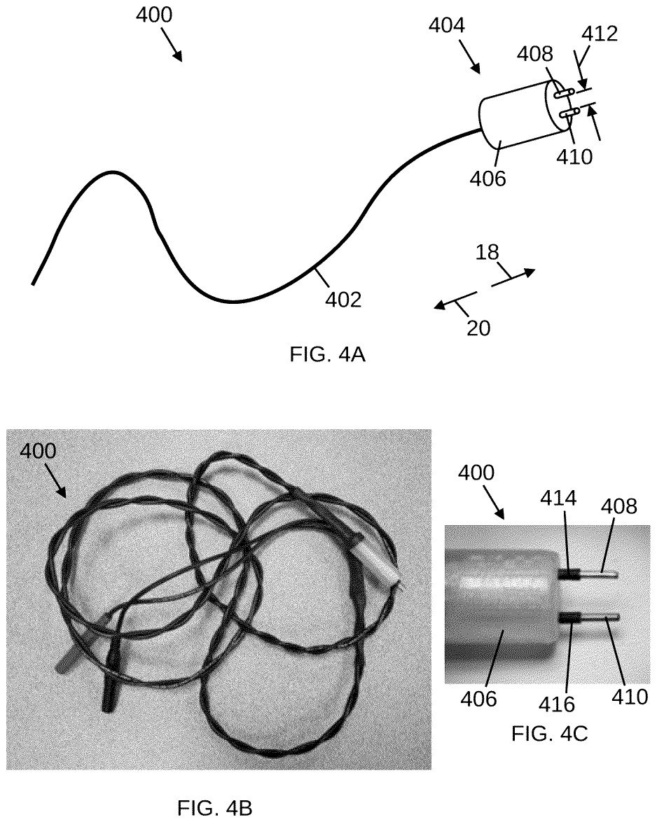

Referring to FIGS. 1 and 4A-4C, the illustrated needle-type PFA device 400 may include an elongated flexible connecting element 402 and/or an end effector 404 disposed distally on the connecting element 402. The connecting element 402 may electrically couple the PFA device 400 to the PFA unit 102. In the illustrated embodiment, the end effector 404, corresponding to end effector 132, may comprise a generally rigid housing 406 and/or one or more electrodes in the form of outwardly extending needles or pins 408, 410 configured to engage the target tissue 10, such as by making a depression therein or penetrating the target tissue 10. In some example embodiments, such penetration may provide desired tissue contact. The electrodes 408, 410, corresponding to electrode 130, may be utilized to deliver PFA energy to the target tissue 10. In the illustrated embodiment, the electrodes are spaced apart at a fixed, inter-electrode spacing 412. In some example embodiments, at least a portion of at least one pin 408, 410 may be covered by an insulator 414, 416, such as a proximal portion of one or more pins 408, 410. In various example embodiments, one or more pins 408, 410 may be generally blunt-tipped and/or generally sharply pointed. Example pin profiles may include short (shallow depth) pins and/or elongated (deep depth) pins, and combinations thereof. In some example embodiments, one or more pins 408, 410 may be in the form of a hollow needle configured to inject a substance into the target tissue. In some such embodiments, a substance may be injected into the target tissue, then PFA energy may be delivered to the target tissue via the needle operating as an electrode.

In view of the context provided by the example embodiments of FIGS. 1, 2, 3A, 3B, and 4A-4C, descriptions of various optional and alternative aspects and features follows.

Generally, PFA devices may be configured for unidirectional and/or bidirectional operation. As used herein, “unidirectional” may refer generally to application of PFA energy to a tissue from one side of the tissue. For example, applying PFA energy to only the epicardial surface of the heart, while not applying PFA energy to the opposed endocardial surface, is an example of unidirectional operation. As used herein, “bidirectional” may refer to application of PFA energy to a tissue from two opposed aspects so that the PFA energy flows through the tissue.

Example unidirectional devices may include needle-type PFA devices, pen-like PFA devices configured to create spot and/or linear lesions, endocardial catheter PFA devices, some minimally invasive, epicardial PFA devices, and/or surface-based end effectors including a plurality of electrodes operated at predetermined, different voltages. Some clamp-type devices, such as those utilizing electrodes on only one jaw, may have a unidirectional configuration.

Example bidirectional devices may include clamp-type PFA devices, graspers, some minimally invasive, epicardial PFA devices, and/or systems configured to place cooperating electrodes on opposing sides of tissue, such as the endocardial and epicardial surfaces of the heart (e.g., using magnetic coupling), or on anterior and posterior surfaces of bodily conduits. Example clamp-type PFA devices may be configured as pinch clamps or no-pinch clamps, and/or may be configured to substantially encircle an anatomic structure (e.g., pulmonary veins) or configured to ablate the wall of hollow organs via insertion of one jaw into a surgical purse string.

Some PFA devices, such as those described above with reference to FIGS. 1, 2, 3A, 3B, and 4A-4C, may include a variety of electrode configurations. Generally, any combination or variation of electrode configurations described herein may be used in connection with any embodiment according to at least some aspects of the present disclosure.

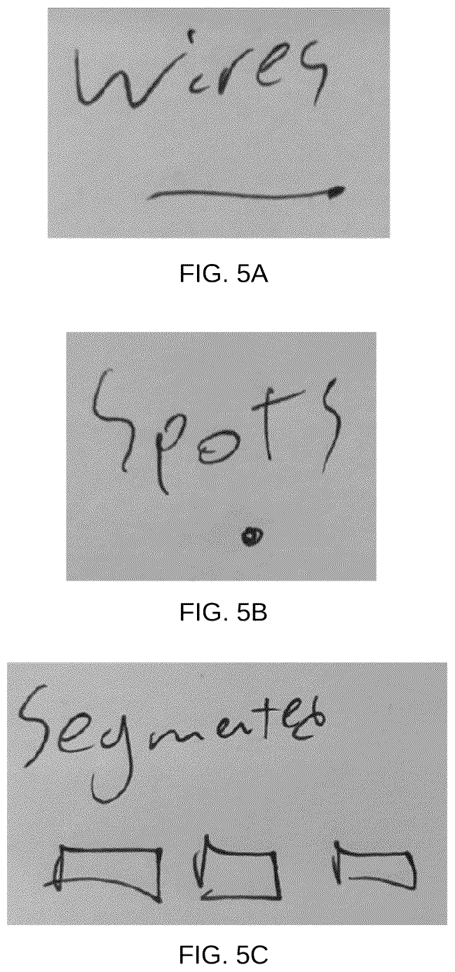

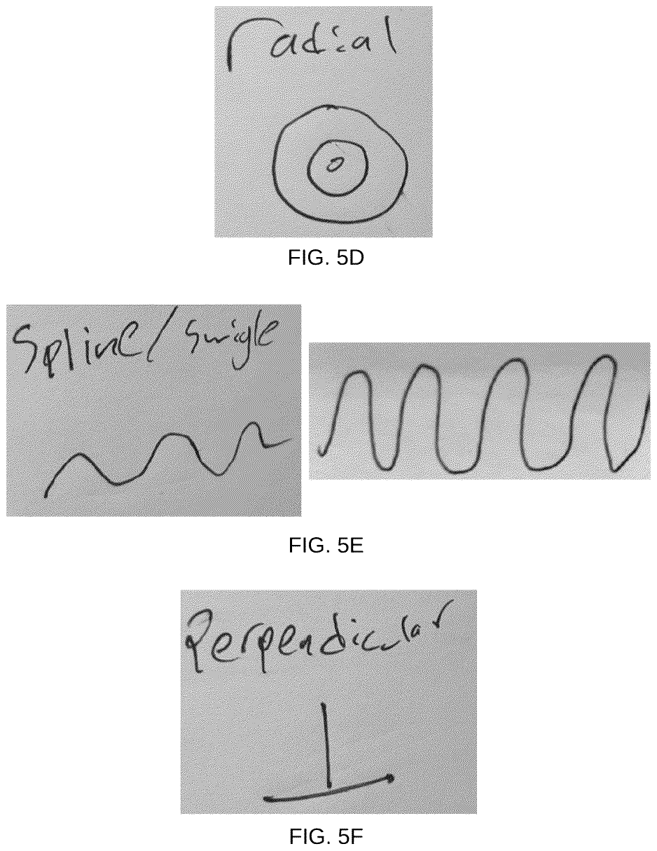

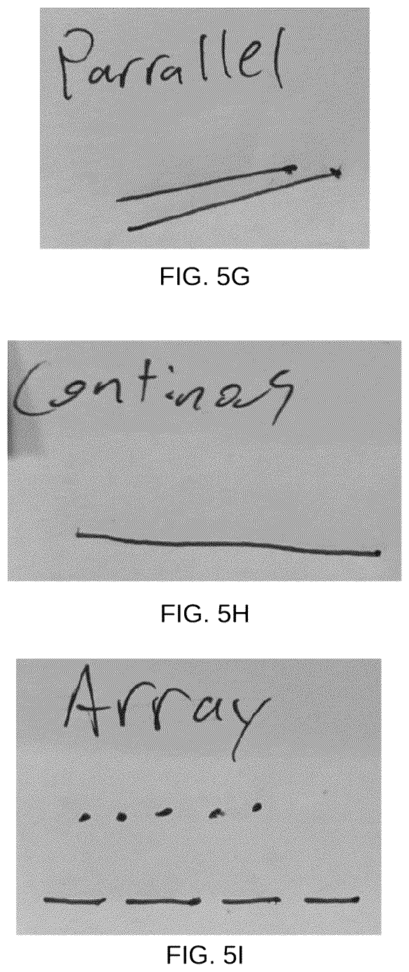

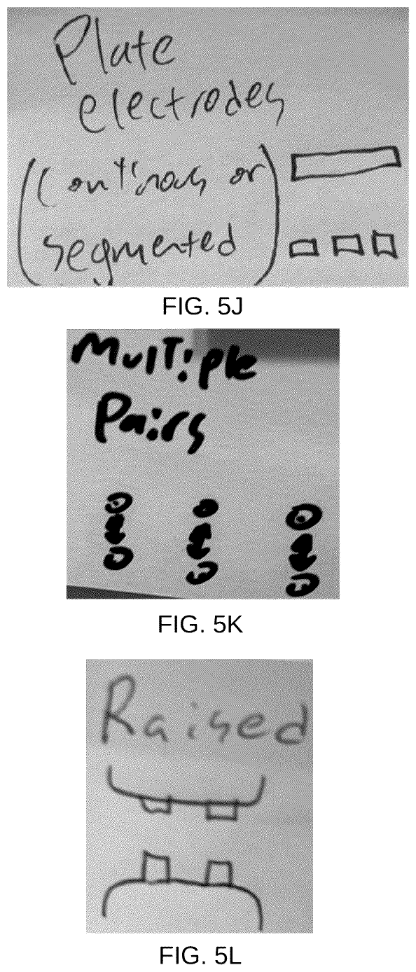

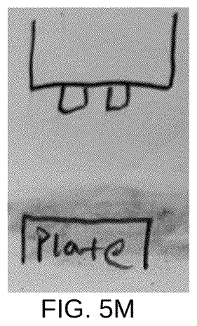

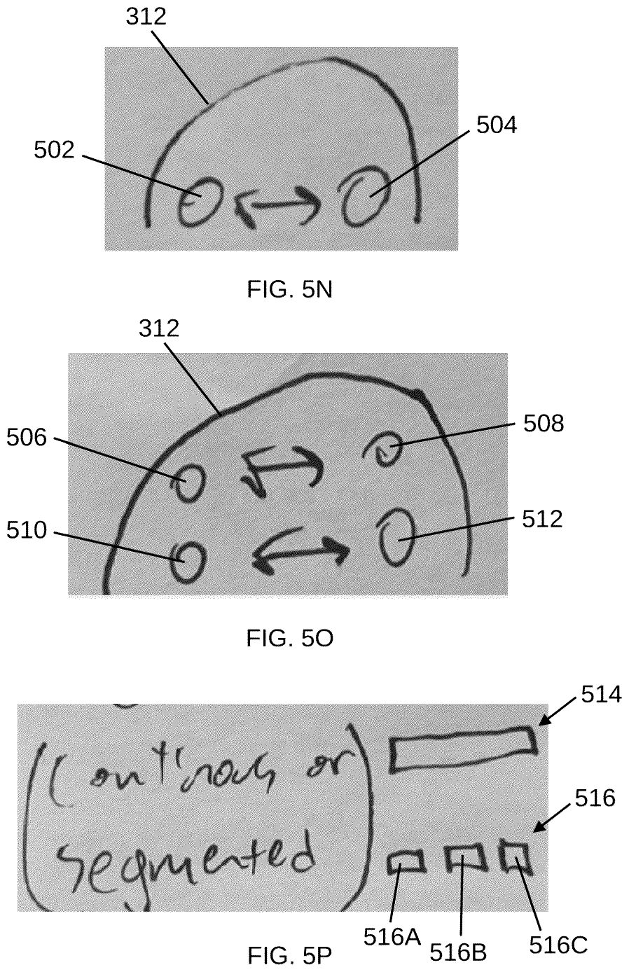

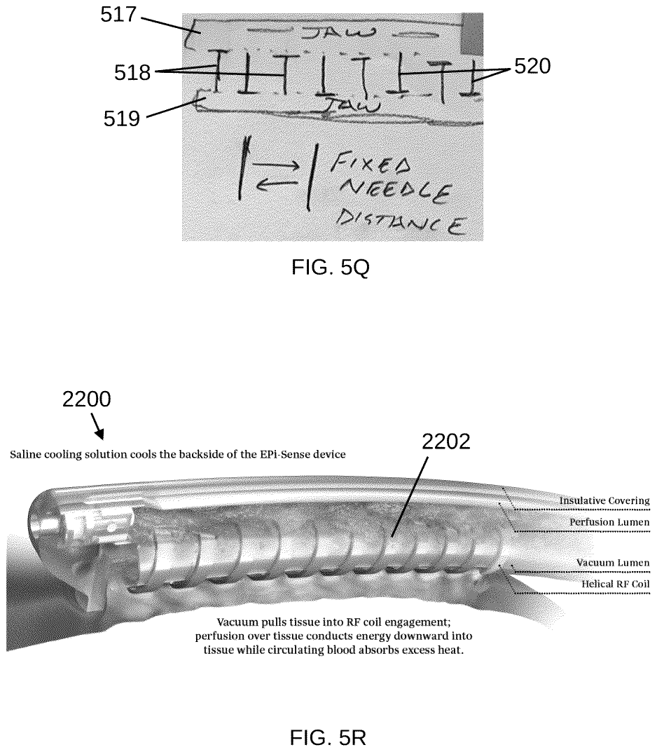

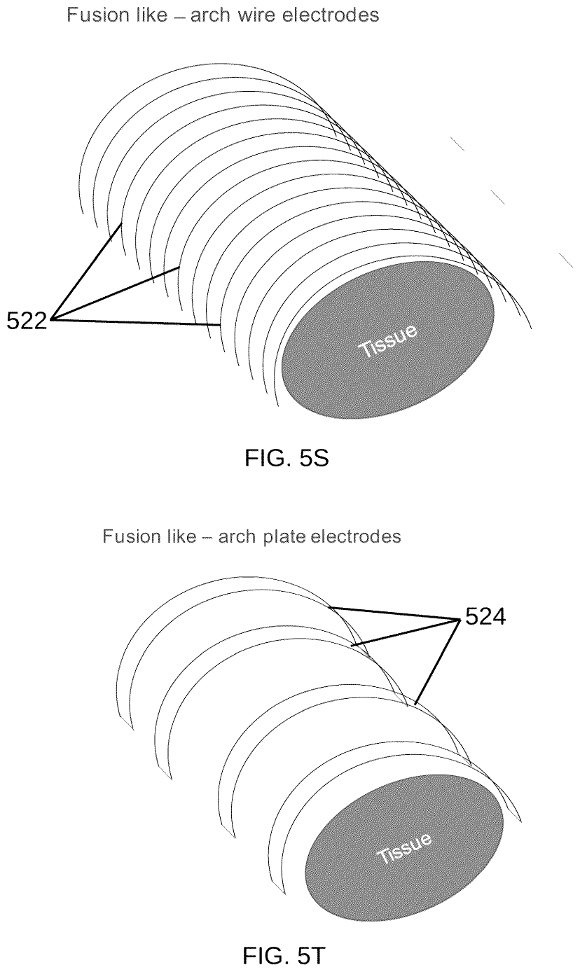

Referring to FIGS. 5A-5T, FIG. 5A illustrates an elongated, generally linear (e.g., “wire”) electrode that may be generally straight, or may include one or more curves and/or angles, and may be repeated to provide multiple electrodes as desired. FIG. 5B illustrates a point (e.g., “spot”) electrode that may be generally circular or may have other shapes, such as a sheathed electrode that exposes a point. FIG. 5C illustrates a segmented electrode that includes a plurality of discreet, generally rectangular segments, though segments with other similar or differing shapes may be utilized. In some example embodiments, the segments may be electrically coupled to one another. Such discreet portions may be arranged in a line, curve, tortuous path, stacked, or other arrangement. FIG. 5D illustrates an example nested electrode arrangement, where one or more electrodes are positioned successively inside another. In the illustrated embodiment, one or more generally annular and/or circular electrodes or electrode segments may be arranged generally concentrically within one another. Nevertheless, other enclosed shapes may be utilized such as, without limitation, triangles, rectangles, pentagons, hexagons, octagons, etc.

FIG. 5E illustrates an example squiggle electrode that includes one or a series of elongated electrodes having a plurality of opposite direction curves that may resemble a sinusoidal curve. FIG. 5F illustrates an example perpendicular electrode arrangement that includes a first segment disposed generally orthogonal to a second segment. The segments may be connected, in the form of a single electrode, or may not be connected, in the form of two separate electrodes or a segmented electrode. In the illustrated embodiment, each segment includes a generally straight electrode. FIG. 5G illustrates an example parallel electrode arrangement that includes a first segment disposed generally parallel to a second segment. In the illustrated embodiment, each segment includes a generally straight electrode. And more than two parallel electrodes may be utilized depending upon the application.

FIG. 5H illustrates an example continuous electrode, while FIG. 5I illustrates two example electrode arrays. Generally, a continuous electrode may have a continuous surface that is presented to a tissue, regardless of the shape of the electrode. Generally, a discontinuous electrode array may include two or more segments having separated tissue contacting surfaces. Each segment may have any shape, such as generally circular and/or generally straight. In some example embodiments, two or more segments of an electrode array may be electrically connected. In some example embodiments, two or more segments of an electrode array may be electrically isolated from each other, so that each delivers differing or the same electrical signals to the tissue, for example.

FIG. 5J illustrates example plate electrodes that may include a two or three dimensional electrode surface having a substantial width in view of its length, regardless of its shape. Plate electrodes may be provided in continuous or segmented configurations, for example. FIG. 5K illustrates an example electrode configuration including multiple pairs of cooperating electrodes. FIG. 5L illustrates example raised electrodes. Generally, raised electrodes may protrude from a surrounding surface of the PFA device. In some embodiments, one or more electrodes may be disposed flush with the surrounding surface. That is, the tissue contacting surface of the electrode may be substantially coplanar with the surrounding surface. In some embodiments, one or more electrodes may be recessed within the surrounding surface. That is, the tissue contacting surface of the electrode may be inset with respect to the surrounding surface. FIG. 5M illustrates an example electrode configuration including raised electrodes positioned opposite a plate electrode.

FIGS. 5N-5T are simplified section views of alternative example electrode configurations which may be used, for example, in connection with PFA devices generally similar to PFA device 200, 300, 400, 2200 according to at least some aspects of the present disclosure. It will be understood, however, the similar configurations may also be utilized in other PFA devices. Specifically, FIG. 5N depicts a longitudinal section view of an example elongated (e.g., wire) electrode configuration, including two electrodes 502, 504. In this embodiment, the elongated electrodes 502, 504 may be arranged as an opposed pair such as for bipolar operation. The electrodes 502, 504 may be oriented generally longitudinally and/or may be at least partially recessed within the stabilizer 312. FIG. 5O depicts a longitudinal section view of an example multiple elongated (e.g., wire) electrode configuration, including four electrodes 506, 508, 510, 512. In this embodiment, the elongated electrodes 506, 508, 510, 512 may be arranged as two opposed pairs and/or may be oriented generally longitudinally. But greater than two pairs of electrodes is within the scope of the instant disclosure. FIG. 5P depicts a lateral section view of a continuous electrode configuration 514 and a segmented electrode configuration 516. In the illustrated embodiment, individual electrodes 516A, 516B, 516C of the segmented electrode configuration 516 may be electrically connected as a group or individually driven, but may individually contact the target tissue.

FIG. 5Q depicts a lateral view of an example electrode arrangement comprising opposed, tissue penetrating needle electrodes. In the illustrated embodiment, a first jaw 517 includes at least one needle electrode 518 extending therefrom. A second, opposed jaw 519 includes at least one needle electrode 520 extending therefrom. In the illustrated embodiment, the needle electrodes 518, 520 are disposed in respective arrays. The spacing between the needles 518, 520 may be fixed at a known, specified distance. While the illustrated embodiment includes jaws 517, 519 that may represent jaws 210, 212 of a clamp-type PFA device (e.g., PFA device 200 depicted in FIG. 2), it will be understood that such an opposed needle arrangement may be utilized with other configurations of PFA devices, such as PFA devices 300, 400.

FIG. 5R depicts a partial cutaway perspective view of the minimally invasive PFA device 2200 illustrated in FIG. 22, where the electrode may be generally in the form of a helical electrode 2202. FIG. 5S depicts a simplified distal perspective view of an example electrode configuration comprising a plurality of arch wire electrodes 522. In this embodiment, the arch wire electrodes 522 may be disposed at least partially within a stabilizer (FIG. 3A) and may be generally parallel, laterally oriented, and/or configured to engage the target tissue on respective concave surfaces. And FIG. 5T depicts a simplified distal perspective view of an example electrode configuration comprising a plurality of arch plate electrodes 524. In this embodiment, the arch plate electrodes 524 may be disposed at least partially within a stabilizer (FIG. 3A) and may be generally parallel, laterally oriented, and/or configured to engage the target tissue on respective concave surfaces. Generally, the arch plate electrodes 524 of FIG. 5T may be similar to the arch wire electrodes 522 of FIG. 5S; however, in some embodiments, the arch plate electrodes 524 may be wider (e.g., in a longitudinal direction) than the arch wire electrodes 522.

In some example embodiments, clamp-type PFA devices (e.g., similar to PFA device 200) may include various features. Generally, clamp-type devices may be configured for dynamic closure and/or static closure.

Example embodiments configured for dynamic closure may utilize static jaws and/or dynamic jaws. For example, a static jaw (e.g., a jaw which does not change orientation during use) may be dynamically configured by use of a spring closure mechanism. In some such embodiments, the closure force is substantially provided by the spring force, and the jaw separation in the closed configuration depends on the tissue thickness and compressibility. In other embodiments, a static jaw may be dynamically configured by utilizing a user-applied closing force. Thus, the jaw separation and closure force in the closed configuration are directly controlled by the user. Example embodiments including dynamic jaws may include a compressible jaw surface, conformable jaws (e.g., jaws that deform when subject to design closure forces), and/or flexible jaws.

Some example embodiments configured for static closure may utilize a pressure set. That is, a closure force may be applied up to a pre-set, desired level. In such a circumstance, a further applied closure force would be ineffective to further close the jaws.

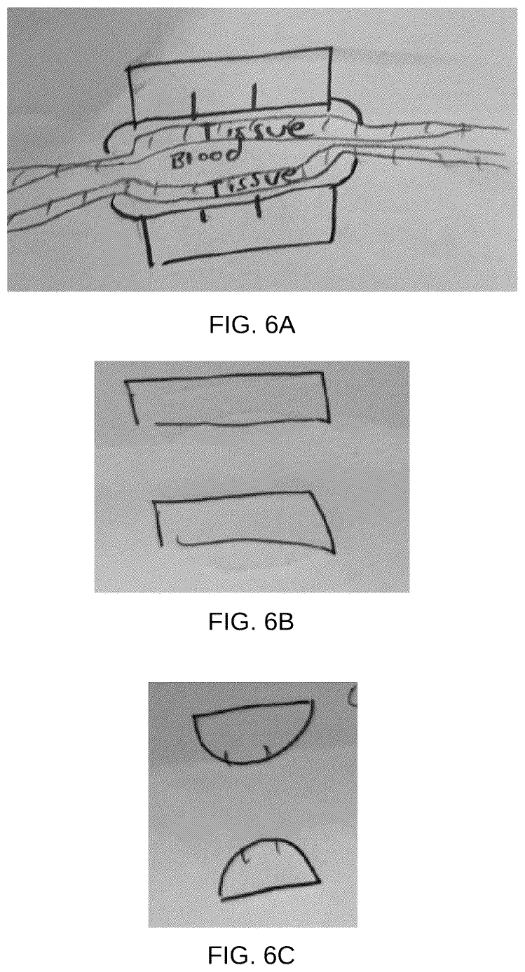

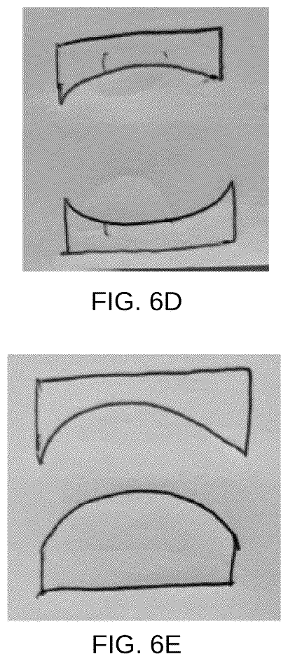

Referencing FIG. 6A, some example embodiments configured for static closure may utilize a fixed distance set. That is, the jaws are closed to a pre-determined jaw spacing, regardless of the closing force necessary to achieve such spacing. In some example embodiments employing a fixed distance set, a PFA device may utilize both clamping and vacuum tissue engagement features. For example, FIG. 6A depicts a simplified section view of a vacuum-clamp configuration, according to at least some aspects of the present disclosure. In the illustrated embodiment, the jaws are positioned around the target tissue and are moved to the closed configuration. Vacuum is applied to the jaws to maintain or increase desired tissue contact with the jaws.

Some example embodiments may be configured for hybrid set distance/dynamic closure operation. For example, an initial closure of a clamp may be performed to a set distance. This may facilitate a PFA, such as at a fixed or known V/cm. Then, the clamp may be closed dynamically, such as in preparation for RF ablation. Some example embodiments may include closure mechanisms that provide such sequences of operations, or that may be switchable (e.g., user-selectable) between such modes of operation. In some example embodiments, a closure mechanism mode of operation (e.g., dynamic vs. fixed distance) may be determined in connection with selecting an output of an electrosurgical generator (e.g., PFA vs. RF ablation). In alternative embodiments, dynamic closure may be performed first, followed by set distance closure, such as to perform RF ablation followed by PFA.

Some example embodiments may utilize a variable distance set. That is, the jaws may be closed to a particular distance, such as may be decided by a user and/or indicated by detents or a visible scale, but that distance may vary from ablation to ablation.

Referring to FIGS. 6B-6E, some example embodiments may utilize opposed jaws including cooperating tissue engagement features. For example, insulator portions of jaws proximate the electrodes may be configured with various shapes for engaging the target tissue. FIG. 6B illustrates a baseline example embodiment including substantially flat opposed tissue engagement surfaces; FIG. 6C illustrates an example embodiment including opposed convex tissue engagement surfaces; FIG. 6D illustrates an example embodiment including opposed concave tissue engagement surfaces; FIG. 6E illustrates an example embodiment including a convex tissue engagement surface opposing a cooperating concave tissue engagement surface; all according to at least some aspects of the present disclosure.

In some example embodiments, the tissue engagement surfaces (e.g., insulators) may be substantially rigid. That is, the insulators do not substantially deform under design loads. In some example embodiments, the tissue engagement surfaces (e.g., insulators) may be substantially compliant. That is, the insulators may be configured to deform, such as to conform to the target tissue, when subjected to design loads. In some example embodiments, the tissue engagement surfaces may be partially rigid and/or partially compliant, as may be suitable to achieve desired tissue contact.

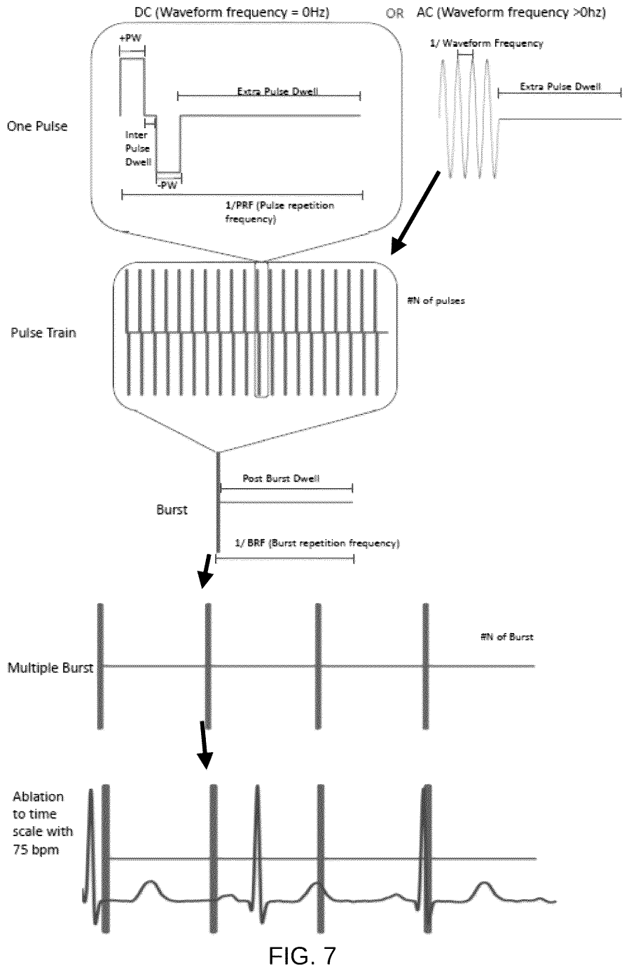

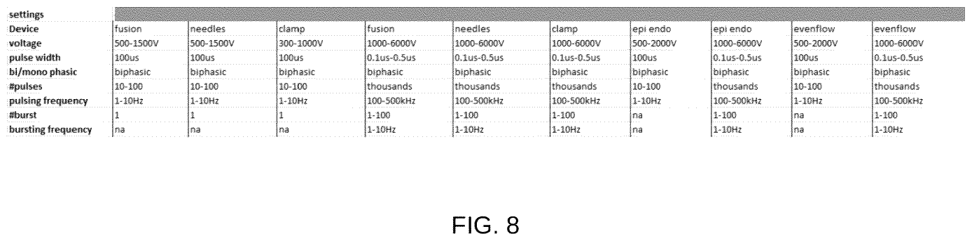

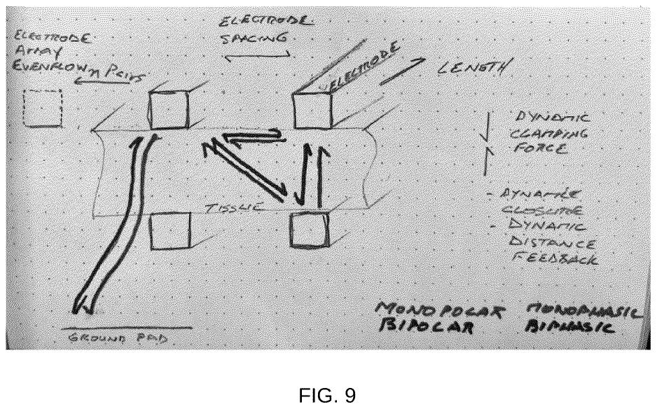

Turning to FIG. 7, a graphical illustration of the composition of an example multiple burst PFA signal is depicted, according to at least some aspects of the present disclosure. And FIG. 8 depicts a table listing example PFA signal parameters that may be used in connection with a variety of PFA devices, according to at least some aspects of the present disclosure. In FIG. 8, “fusion” refers to a device similar to PFA device 300 shown in FIG. 3, “needles” refers to a device similar to PFA device 400 shown in FIGS. 4A-4C, “clamp” refers to a device similar to PFA device 200 shown in FIG. 2, “epi endo” refers to a PFA system comprising an epicardially positioned PFA device operated in cooperation with an endocardially positioned PFA device, and “evenflow” refers to a PFA device comprising a plurality of electrodes operated at predetermined, different voltages. The listed parameters are merely examples and should not be considered limiting in any way.

In various example embodiments, PFA signals may comprise monophasic pulses and/or biphasic pulses. Individual pulses may comprise square waves and/or voltage may vary over time. For example, an individual pulse may comprise a generally sinusoidal waveform. Individual pulses may be delivered in a burst (e.g., a pulse train). A series of multiple bursts may be delivered. Some example embodiments may deliver pulses at particular predetermined times relative to a patient's heartbeat.

Pulse characteristics may be varied and selected to achieve a desired result. For example, alternating current (“AC”) or direct current (“DC”) waveform, pulse amplitude, number of pulses in a pulse train, number of bursts, pulse repetition frequency, burst repetition frequency, pulse width (e.g., nanosecond or greater), etc., may be varied. In some example embodiments, some or all of the characteristics may remain substantially constant. In some example embodiments, one or more characteristics may change, such as during the course of an ablation. For example, some characteristics may be programmed to change with time.

In some example embodiments, operation of a PFA system may be configured to measure one or more parameters, in real-time or time delayed, associated with an ablation operation and/or to utilize data pertaining to such parameters in connection with controlling the delivery of PFA energy. In some example embodiments, based at least in part upon detection and/or measurement of some parameters, some aspects of the PFA energy delivery may be enabled and/or inhibited. In some example embodiments, one or more aspects of a PFA energy delivery may be adjusted and/or controlled based at least in part upon detection and/or measurement of one or more parameters.

In some example embodiments, contact force, or a parameter associated with the contact force, between an end effector and a target tissue may be measured. For example, in an embodiment employing vacuum stabilization, vacuum level may be measured. In an embodiment employing magnetic attraction, the magnetic attraction force may be measured.

In some example embodiments in which the spacing between electrodes may vary, such spacing may be measured as described elsewhere herein.

In some example embodiments, one or more temperatures may be measured. For example, one or more end effector temperatures, electrode temperatures, and/or tissue temperatures may be measured.

In some example embodiments, tissue conductance may be measured. For example, tissue conductance may be measured using the same electrodes as may be used to deliver PFA energy. Alternatively, additional electrodes, different from those used to deliver PFA energy, may be used to measure tissue conductance. Tissue conductance measurements may be assessed as absolute conductance values and/or in view of a change in conductance, such as a percent conductance change resulting from an ablation. In some circumstances, tissue conductance may increase due to PFA, so such measurements may facilitate assessment of ablation effectiveness and/or progress.

In some example embodiments, current delivered in connection with PFA may be measured.

In some example embodiments, ablation time may be measured.

The present disclosure contemplates that tissue selectivity, which may refer to the ability to target destruction of specific tissues with minimal damage to other non-targeted nearby tissues, may be a relevant consideration when ablating tissues. For example, ablation of myocardial tissue may occur near the phrenic nerve, the esophagus, and coronary arteries.

The present disclosure contemplates that tissue selectivity of PFA may be influenced by various factors, including duration and intensity of the electric field, shape and size of electrodes, and electrical properties of the target tissue. Generally, PFA may be more selective for tissues with higher electrical conductivity, such as myocardium, and less selective for tissues with lower conductivity, such as fatty or fibrous tissue. In some circumstances, PFA energy delivered via electrical signals with certain characteristics may be substantially destructive to myocardium and/or may be minimally destructive to nerve tissue and/or blood vessels.

The present disclosure contemplates that, generally, electric field strength (E) (also referred to as applied electric field) is proportional to the voltage (V) applied and inversely proportional to the electrode spacing (d), as given by the following equation:

$$E = \frac{V}{d}$$

The electrode spacing (d) is defined as the distance between electrodes used to deliver the high-voltage electric pulses to the target tissue. In some example embodiments, IRE may be produced by electric field strengths of about 2500 V/cm to 10,000 V/cm.

The present disclosure contemplates that the electrode spacing may affect the spatial distribution of the electric field within the tissue. Specifically, as the electrode spacing increases, the electric field may become less concentrated given its distribution over a larger area, while at smaller electrode spacings, the electric field may become more focused given its concentration in specific regions of the tissue.

The present disclosure contemplates that electrode exposure may also affect the spatial distribution of the electric field within the tissue. Electrode exposure refers to the amount of electrode surface area that is in direct contact with the tissue being treated. Generally, a larger electrode exposure may result in a more uniform electric field distribution across tissue, which can lead to more effective destruction of the targeted tissue. In contrast, a smaller electrode exposure may result in a more localized electric field distribution within a smaller tissue footprint. The electrode exposure can be controlled by adjusting the size and shape of the electrode and/or by varying the distance between the electrode and the tissue. In some cases, multiple electrodes (greater than two) may be used to achieve a larger electrode exposure and/or a more uniform electric field distribution.

The present disclosure contemplates that pulse width may play an important role in determining the effectiveness of a PFA treatment. Pulse width may refer to the time during which the electric field is applied to the tissue. Generally, a longer pulse width may correspond to increased likelihood of causing IRE. However, in some circumstances, too short of a pulse width may deliver insufficient energy to the tissue to produce the desired effect.

The present disclosure contemplates that dwell may determine the amount of energy delivered to the tissue during PFA and thus may affect the extent of tissue damage. Dwell may refer to the time between individual pulses and/or the time between a packet or group of pulses.

The present disclosure contemplates that pulse repetition frequency may affect the duration and/or frequency at which the tissue is exposed to the electrical field, which may impact efficacy, selectivity, and/or safety, in some circumstances. Generally, pulse repetition frequency refers to the frequency at which electric pulses are delivered during PFA. In some circumstances, increasing the pulse repetition frequency may increase selectivity and reduce the likelihood or magnitude of undesired muscle stimulation.

The present disclosure contemplates that the number of pulses delivered to the tissue may affect the extent of tissue destruction and/or the effectiveness of the treatment. The number of pulses may refer to the total number of discrete times that high-voltage current is applied to a target tissue during a particular treatment. Generally, reducing the number of pulses for a given high-voltage current may reduce the potential for undesired heating of tissue.

Referring to FIG. 9, the following description of bipolar/monopolar configurations and biphasic/monophasic signals according to at least some aspects of the present disclosure is explained.

The terms “bipolar” and “monopolar” may refer to the electrical configuration of the electrodes used to deliver the high-voltage electric pulses to the tissue being treated. Generally, in a monopolar configuration, a single active electrode (or group of electrodes) may be used to deliver the electric pulses to the tissue, while another electrode and/or grounding pad is typically placed elsewhere on the patient's body to complete the circuit. Such a configuration may result in a less controlled electric field distribution and/or may potentially damage healthy tissue in the vicinity of the treatment area. In a bipolar configuration, two active electrodes (or groups of electrodes) may be placed near the tissue being treated, with the high-voltage electric pulses being delivered between the two electrodes. Such a configuration may result in a more localized electric field distribution, which may reduce the risk of damage to healthy tissue outside the treatment area. Although both bipolar and monopolar configurations have been used in PFA, in some circumstances, bipolar configurations may provide some potential safety and/or effectiveness advantages. For example, in some circumstances, bipolar configurations may provide improved electric field control and/or more controlled lesion formation and/or may cause less skeletal muscle stimulation.

The terms “biphasic” and “monophasic” may refer to the waveform of electric pulses used in PFA treatment. A monophasic pulse may include a single, high-voltage electric field that is applied to the tissue for a short duration. Such a pulse may be thought of as a unidirectional wave that propagates through the tissue. A biphasic pulse may include two pulses of opposite polarity that are applied in succession. The polarity of the electric field is reversed between the two pulses, resulting in a bidirectional waveform that oscillates back and forth through the tissue. In some circumstances, biphasic pulses may be more effective in disrupting the cell membranes of some tissues, as compared to monophasic pulses.

The present disclosure contemplates that, in some circumstances, delivering electrical energy to bodily tissues may result in muscle contraction. In some circumstances, muscle tissue may contract due to direct stimulation by the electrical energy. In some circumstances, muscle tissue may contract due to stimulation of nervous tissue by the electrical energy. In the context of electrical ablation of cardiac tissue, electrical energy may cause stimulation of cardiac muscle and/or stimulation of non-cardiac, skeletal muscle. For example, such stimulation may include involuntary contractions and/or twitching.

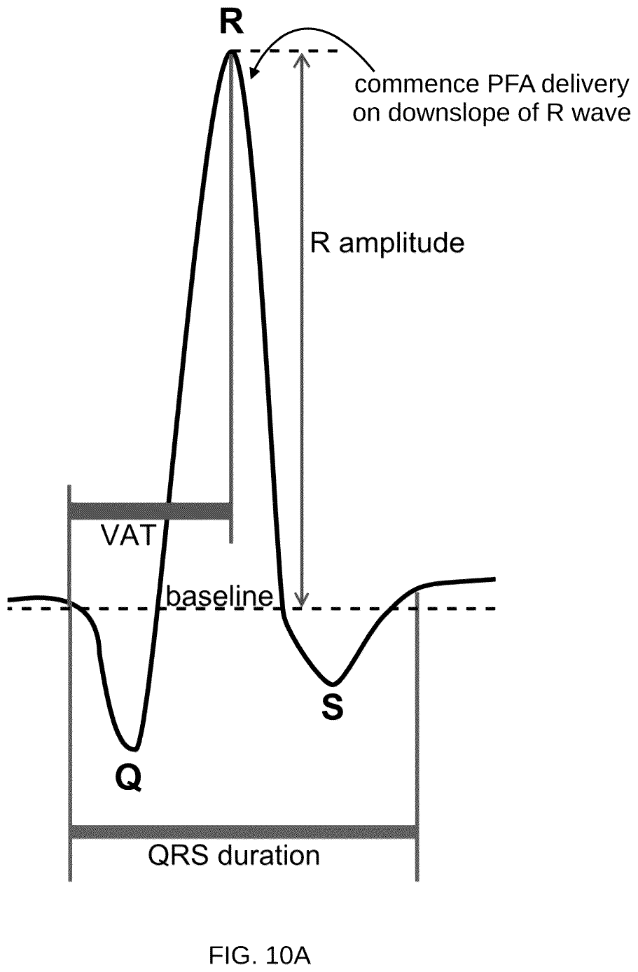

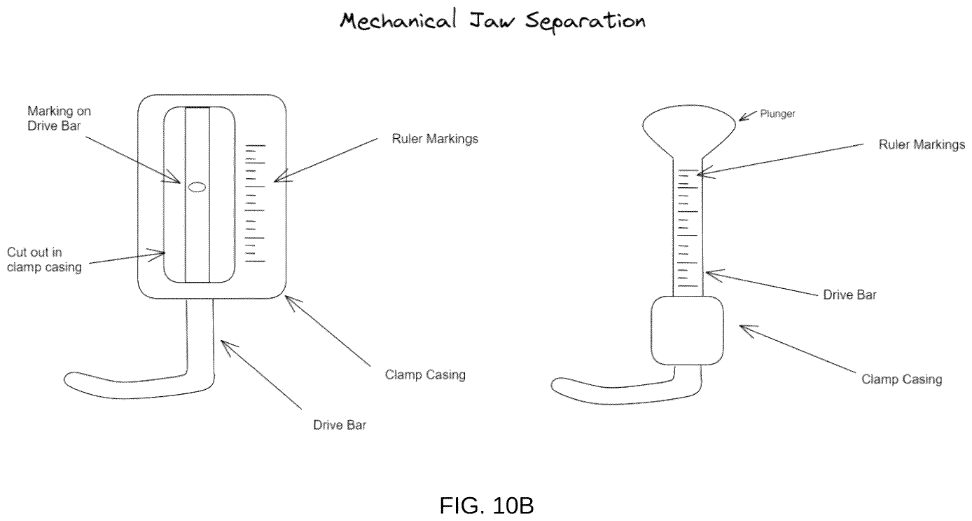

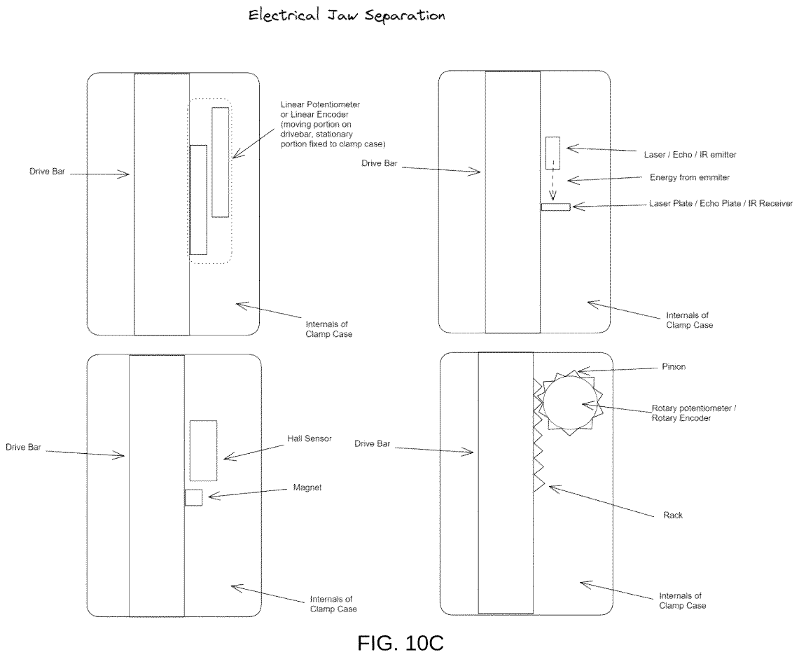

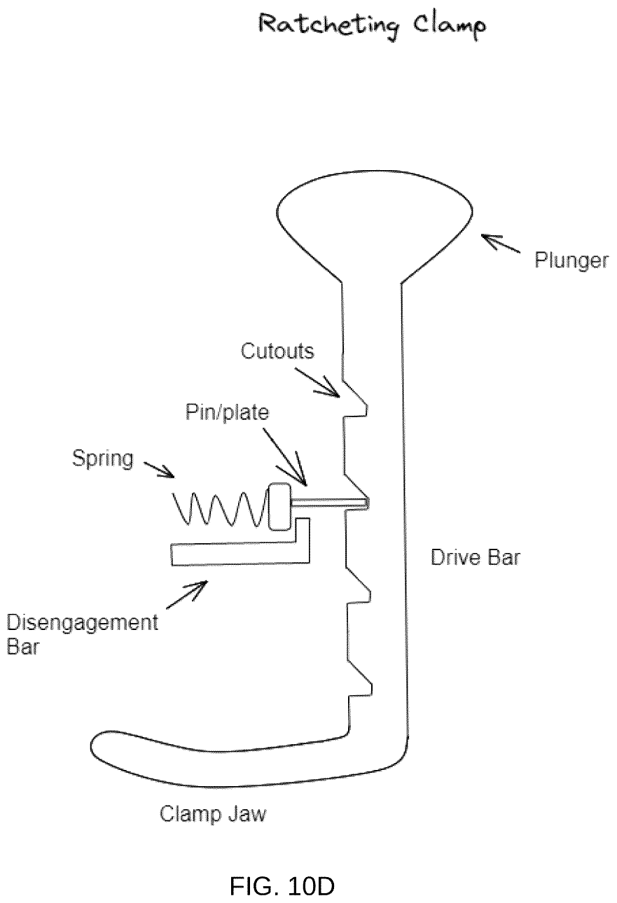

In some example embodiments, delivery of PFA energy to cardiac tissue may be timed to coordinate with the patient's heartbeat. For example, delivery of PFA energy may be timed to align with specific portions of the cardiac cycle and/or not to align with specific portions of the cardiac cycle. For example, electrical energy may be applied beginning when the heart is in its refractory period, which may reduce the likelihood of muscle spasm. For instance, as depicted in FIG. 10, a plot of an example ECG trace according to at least some aspects of the present disclosure is disclosed. In some embodiments, PFA energy delivery may commence on the downslope of the R-wave, which may reduce the likelihood of undesired, abnormal heart stimulation and/or arrythmia. Specifically, on the downslope of the R-wave, the cells have already depolarized, so they are generally unable to react to the PFA signal. In some circumstances, by applying the first of a series of pulses on the downslope of the R-wave, subsequent pulses may be applied at differing frequencies without causing adverse effects. In particular, regardless of the frequency of the subsequent pulses, the heart may be stimulated to beat at a maximum rate of about five beats per second and is likely to return to normal sinus rhythm after the ablation.

In some example embodiments employing pacing signals to drive the heart at a known rate, PFA energy delivery may be timed to coordinate with pacing signals.

In some example embodiments, PFA energy delivery parameters may be selected to reduce the likelihood of undesired cardiac and/or skeletal muscle stimulation. For example, delivering electrical energy at frequencies of about 100 kHz or greater may result in less muscle stimulation.

The present disclosure contemplates that arcing may occur between electrodes at some voltages used for PFA, leading to undesired tissue burns, cardiac arrest, hearing loss, blindness, nerve damage, and/or death depending upon the placement of PFA electrodes. In other PFA circumstances, arcing may occur between an electrode of a PFA device and the patient's tissue (e.g., target tissue or non-target tissue). Accordingly, mitigation of unintended arcing may be a consideration for the design and operation of PFA systems.

In some example embodiments, arcing may be reduced by ensuring sufficient contact force or pressure between a PFA electrode and a target tissue. For example, some embodiments may utilize vacuum stabilization to increase contact pressure. The vacuum pod may include fluid flow with conductive fluid to ensure tissue to electrode coupling. Some embodiments may utilize a clamp-type configuration to increase contact pressure. Some embodiments may utilize an expandable structure to increase contact pressure.

In some example embodiments, arcing may be reduced by covering, irrigating, and/or submerging electrodes and/or tissues in a dielectric fluid, such as deionized water.

In some example embodiments comprising a plurality of electrodes, one or more electrodes may be selectively activated and/or disabled. For example, one or more electrodes in contact with a target tissue may be activated and/or one or more electrodes not in contact with the target tissue may be disabled/deactivated in connection with a particular application of PFA energy. For example, one or more electrodes not contacting any tissue and/or one or more electrodes in contact with tissue other than the target tissue may be disabled. The electrodes may be manually disabled by the user or automated by electrical contact testing with applied voltage to confirm tissue contact or by any combination thereof. In some example embodiments, some electrodes may be selectively utilized for particular ablation modalities (e.g., PFA, RF).

In some example embodiments including PFA devices comprising two or more electrodes, a PFA device may be constructed so that the spacing between adjacent electrodes is sufficient to avoid arcing at a desired voltage. For example, given a maximum voltage differential between two adjacent electrodes, a minimum spacing may be determined that avoids arcing. In some embodiments, such spacing may be fixed when the device is constructed by rigidly disposing the electrodes at a desired spacing. In some embodiments, such as clamp-type devices with electrodes disposed on opposed jaws, mechanical controls may be incorporated that limit the closing movement of the jaws to a minimum separation distance providing sufficient electrode spacing to avoid arcing, and/or incorporation of electrical controls to inhibit operation of the electrodes if the electrode spacing is insufficient. Such controls may be disposed in the end effector, acting on or close to one or more jaws, and/or in a handle, acting on or close to a user-operated actuation element, and/or in the PFA unit 102 as physical circuitry and/or programming code.

In some example embodiments, potential arcing conditions may be detected and/or prevented. For example, a PFA device and/or PFA unit may be configured to prevent delivery of PFA energy when potential arcing conditions are detected. Alternatively, a PFA device and/or PFA unit may be configured to adjust its operation based upon detection of potential arcing conditions. For example, electrodes not in contact with tissue may be disabled, thus allowing application of PFA energy only through electrodes in substantial contact with tissue. In some example embodiments, parameters associated with arcing conditions may be detected and energy delivery may be terminated. For example, if voltage, current, conductivity, impedance, or other electrical parameters associated with arc initiation are detected, an example PFA unit 102 may terminate energy delivery to the PFA device.

Some example embodiments allowing variable electrode spacing (e.g., at least one electrode that is repositionable relative to at least one other electrode) may be configured to measure the electrode spacing, such as before PFA energy is delivered to the electrodes. In some clamp-type embodiments, determining the jaw separation distance may correlate with the electrode spacing. For example, if it is determined that the electrode spacing is insufficient to prevent arcing at a desired voltage, the PFA system may prevent delivery of PFA energy to the electrodes. In some example embodiments, a maximum voltage delivered to the electrodes may be adjusted based at least in part upon a detected or determined electrode spacing. That is, for example, the maximum voltage may be lower when a closer electrode spacing is detected or determined and/or the maximum voltage may be higher when a farther electrode spacing is detected or determined.

In some clamp-type PFA devices, jaw separation may be controlled and/or determined mechanically and/or electrically. Example mechanical configurations may include mechanisms configured to measure the distance between the opposed jaws, a ratcheting mechanism associated with the distance between the jaws, a window cut-out on the shaft or handle indicating the separation between the jaws, and/or ruler marks on the shaft, for example. Example electrical configurations may include magnet and Hall sensor devices, linear potentiometers, laser, echo, infrared, and/or tissue impedance.

Turning to FIG. 10B, two example embodiments configured to mechanically measure the distance between opposed jaws are illustrated. As illustrated, in some embodiments, ruler markings may be provided on a component that is stationary relative to a moving drive bar, or may be provided on a moving drive bar, for example.

Referring to FIG. 10C, four example embodiments configured to electrically and/or electronically measure the distance between opposed jaws are illustrated. As illustrated, some embodiments may include linear potentiometers (or linear encoders), Hall sensors, laser/echo/or IR distance measurement, and/or rotary potentiometers (or encoders), for example.

The present disclosure contemplates that voltages associated with PFA may be substantially greater than voltages associated with RF ablation. Accordingly, PFA devices may utilize increased electrical insulation as compared to RF-only devices. For example, it may be advantageous to construct PFA devices from nonconductive materials and/or to insulate (or increase insulation on) conductive elements of PFA devices. As an example, it may be advantageous to electrically insulate a tubular metal shaft extending between a handle and an end effector.

In some example embodiments, the material(s) from which electrodes are constructed may be selected to reduce the likelihood of arcing. For example, some electrodes may be constructed entirely of a single material having certain characteristics. In some example embodiments, a portion of an electrode (e.g., a main body portion) may be constructed from a first material and may be at least partially covered (e.g., plated or coated) with a second material, different than the first material. Example electrode materials include, without limitation, copper, gold, and nickel.

In some example embodiments, one or more electrodes may be formed to reduce the likelihood of arcing. For example, in some embodiments, relatively large radius curved edges may be less likely to arc than sharp corners and/or pointed projections. In some example embodiments, an array of a plurality of relatively smaller electrodes may be less likely to arc than a single, relatively larger electrode.

Turning to FIG. 10D, various clamp-type PFA devices according to at least some aspects of the present disclosure may utilize a ratcheting clamp mechanism. One such example of a ratcheting clamp mechanism is depicted. In the illustrated embodiment, the mechanism, specifically the interaction between the cutouts and the pin/plate, may be configured to allow the plunger, drive bar, and clamp jaw to move in a closing direction, while preventing movement in an opening direction. The disengagement bar may be used to withdraw the pin/plate from the cutouts, thus allowing the clamp jaw to be moved in the opening direction, when desired.

In some example embodiments, a PFA device may include one or more insulator and/or electrode arrangements configured to reduce the likelihood of arcing. Although the following example features are illustrated and described individually in the context of a clamp-type PFA device 200, one or more similar features may be utilized in connection with any PFA device configuration, including those generally similar to minimally invasive PFA device 300.

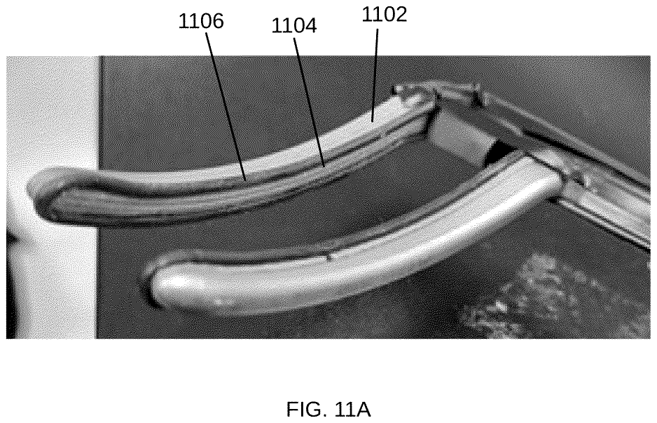

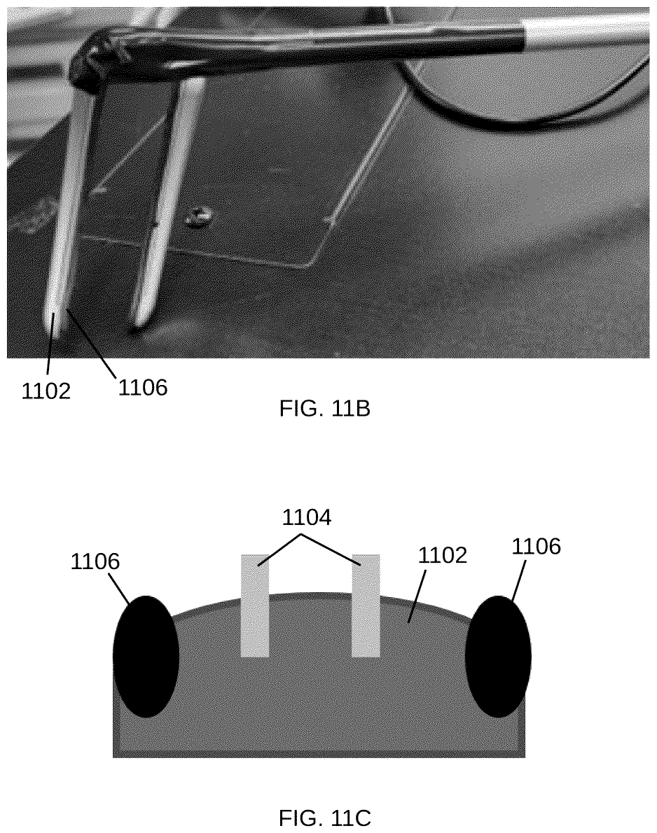

Referring to FIGS. 11A and 11B, an example insulator configuration comprising a compressible insulator at least partially circumscribing one or more electrodes is depicted. FIG. 11C is a simplified section view of the embodiment of FIGS. 11A and 11B. In the illustrated embodiment, a jaw 1102 may include one or more electrodes 1104, configured to deliver PFA energy, disposed on a tissue-engaging surface. One or more compressible insulators 1106 may at least partially circumscribe the electrodes 1104. In the illustrated embodiment, the compressible insulator 1106 may be formed from flexible, compressible, insulative tubing affixed to the perimeter of the jaw 1102. When an object (e.g., tissue) is clamped between the jaws 1102, the compressible insulators 1106 are deformed (see FIG. 11C) so that the electrodes 1104 effectively protrude from the jaw substrate and make direct contact with the object clamped. In contrast, when an object is not clamped between the jaws 1102, the compressible insulators 1106 protrude from the jaw substrate and extend outward beyond the reach of the electrodes 1104 to mitigate accidental electrical discharge to unintended objects. Insulators 1106 may be set to a height to ensure that the electrodes 1104 are never in direct contact, or to ensure a minimum electrode separation is maintained. The durometer of 1106 may be selected to achieve a specific rate of compression, that could be relatively stiffer or softer than the tissue clamped between the opposing jaws.

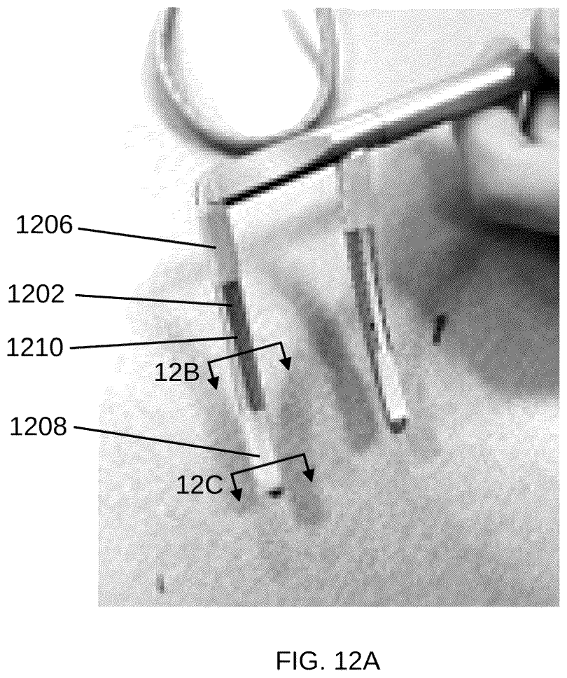

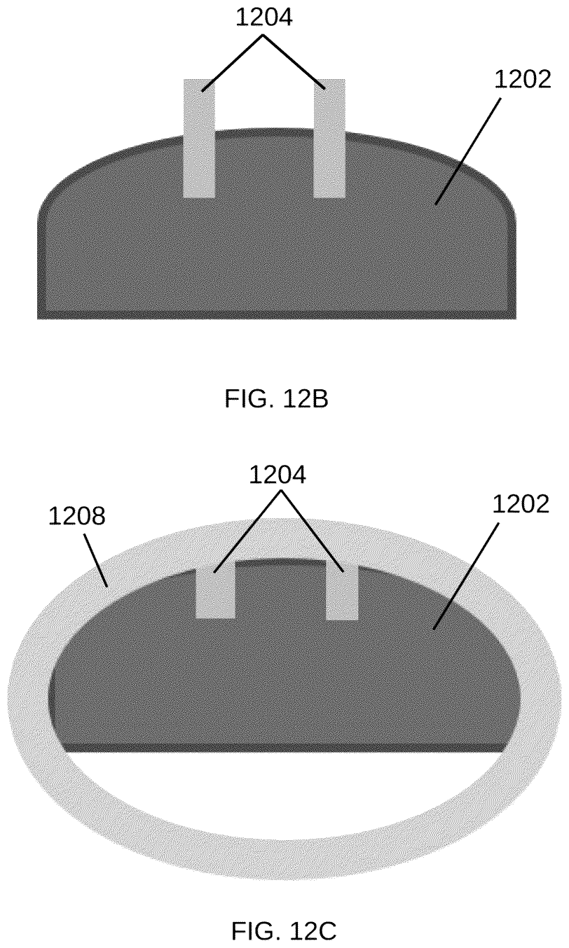

Turning to FIG. 12A, an example insulator configuration forming a shortened electrode exposure region is depicted. FIGS. 12B and 12C are simplified section views of the embodiment of FIG. 12A. In the illustrated embodiment, a jaw 1202 may include one or more electrodes 1204 configured to deliver PFA energy disposed on a tissue-contacting surface. One or more portions of the electrodes 1204 may be at least partially covered by a fixed or adjustable (sliding) insulator so that the tissue-contacting length of the electrodes is reduced. In the illustrated embodiment, a first insulator 1206 may cover a portion of the jaw 1202 proximate the heel and/or a second insulator 1208 may cover a portion of the jaw 1202 proximate the toe. Accordingly, a generally central portion 1210 of the jaw 1202 may remain uncovered, thus allowing contact between the electrodes and the target tissue. In the illustrated embodiment, the first and second insulators 1206, 1208 may be formed from silicone tape wrapped around the jaw 1202. It should be noted that insulators other than silicone may be utilized to inhibit direct contact between a portion of the electrodes 1204 and tissue.

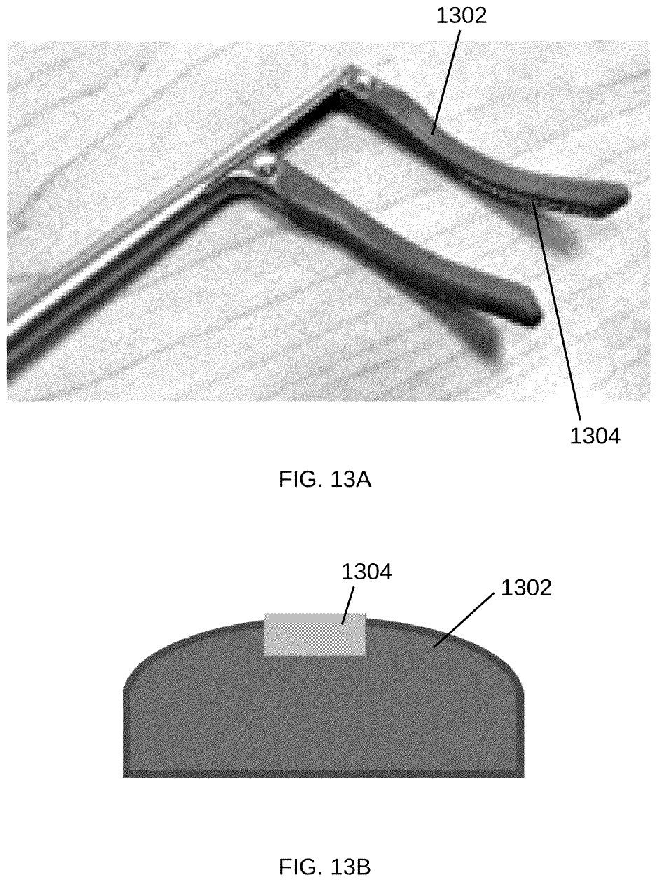

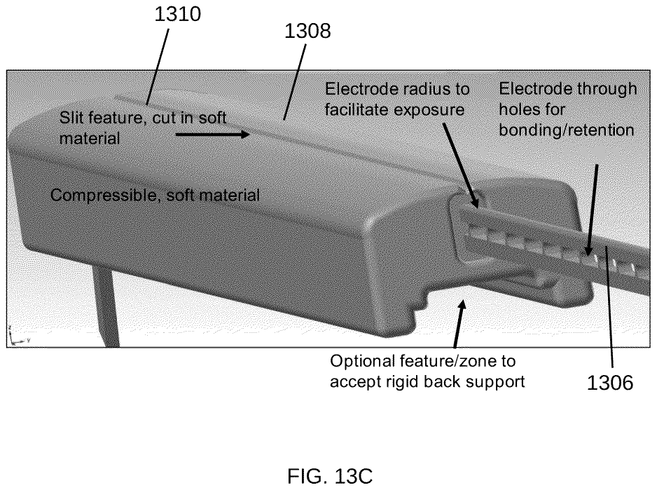

Referring to FIG. 13A, an example configuration comprising an insulated jaw is depicted. FIG. 13B is a section view of the embodiment of FIG. 13A. In the illustrated embodiment, a jaw 1302 may be constructed from non-conductive (e.g., insulative) material(s). An electrode 1304 may be embedded therein and/or disposed thereon. Thus, in contrast to embodiments in which a jaw includes externally exposed conductive material(s), the risk of arcing between an electrode and the jaw may be reduced.

Some example embodiments according to at least some aspects of the present disclosure may include one or selectively exposed electrodes. For example, some embodiments may include one or more electrodes that may be at least partially covered by one or more relatively soft, deformable insulators. In some example embodiments, one or more insulators may be configured to elastically deform and/or move to at least partially expose one or more electrodes to allow contact between the electrodes and a target tissue. Generally, in some embodiments, the electrodes may remain at least partially covered by the insulators when not in contact with the target tissue. In some example embodiments, insulators may be constructed from soft, compressible materials. The material properties may be selected so that the material moves (e.g., at least partially exposing an electrode) when clamped on tissue. In some alternative embodiments, insulators may be constructed from materials with self-healing properties.

Some example embodiments, may be constructed with one or more slits configured to facilitate elastic movement and/or electrode exposure. In various embodiments, electrodes may have any shape, including shapes configured to facilitate exposure. For example, some electrodes may be generally round, generally rectangular, generally tear-drop shaped, generally parabolic, etc.

In some example embodiments, insulators may be overmolded onto electrodes. For example, an insulator may be overmolded onto and/or bonded to a generally smooth, wire electrode. In some example embodiments, an electrode may include a coupling feature, such as a transverse through-opening, configured to facilitate bonding and retention between the insulator and the electrode. In some example embodiments, electrodes may be inserted into openings in insulators.

Some example embodiments may include a relatively rigid backing support provided within or proximate a relatively soft insulator. For example, a backing may be in the form of a flat plate and/or a grooved block, which may reduce electrode rolling and/or twisting. Some example embodiments may include intermittent post supports configured to support an electrode relative to an underlying, relatively rigid structure (e.g., jaw).

Referencing FIG. 13C, an alternative embodiment in which an electrode 1306 is selectively insulated is illustrated. In some example embodiments, the configuration of FIG. 13C may be utilized in place of the exposed electrode configuration of FIG. 13B. In the illustrated embodiment, an insulator 1308, which may be constructed of an insulative, deformable material, may be arranged to at least partially cover the electrode 1306. In a manner as generally as described below with reference to other selectively insulated electrodes, the insulator 1308 may be deformable to at least partially expose the electrode 1306, such as upon engagement with a target tissue. In the illustrated embodiment, the insulator 1308 may include a generally longitudinal slit feature 1310, which may facilitate selective exposure of the electrode 1306.

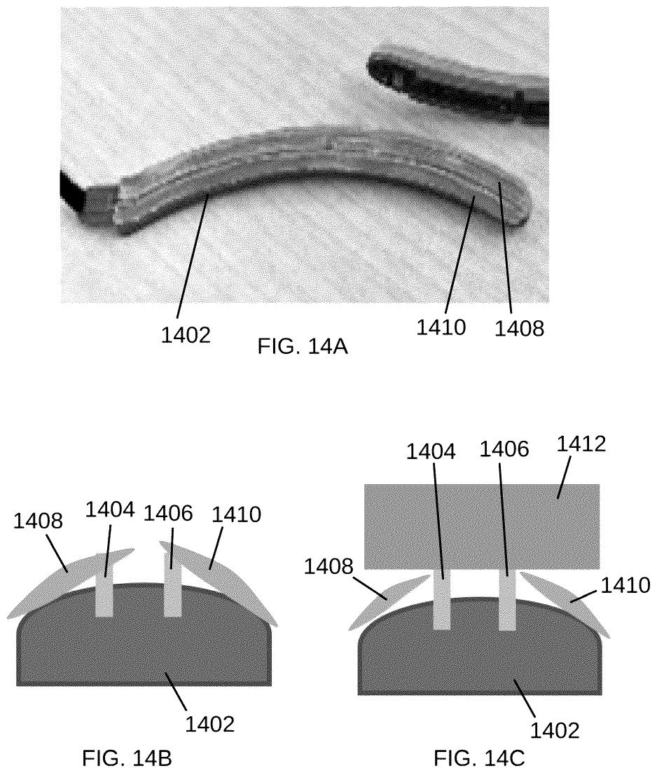

Turning to FIG. 14A, an example configuration comprising a jaw with selectively insulated electrodes is illustrated. FIGS. 14B and 14C are section views of the embodiment of FIG. 14A with the electrodes in contact with the target tissue. In the illustrated embodiment, a jaw 1402 may include one or more electrodes 1404, 1406 on a tissue-engaging surface. Referring to FIG. 14B, one or more deformable insulators 1408, 1410 may at least partially cover the one or more electrodes 1404, 1406 when the jaw 1402 is not in contact with tissue. In some example embodiments, the insulators 1408, 1410 may be constructed from flexible silicone or other suitable materials, for example. In the illustrated embodiment, each insulator 1408, 1410 substantially covers the entire length of a respective electrode 1404, 1406, with a central slit running along the length of the jaw 1402 between the inner aspects of the insulators 1408, 1410. Referring to FIG. 14C, when the jaw is placed into contact with the target tissue 1412, the target tissue 1412 may deform the insulators 1408, 1410 (e.g., generally laterally) to expose the electrodes 1404, 1406, thus allowing the target tissue 1412 to contact the electrodes 1404, 1406. If the target tissue extends substantially the full length of the jaw, substantially the full lengths of the electrodes 1404, 1406 may be exposed for contact with the target tissue 1412. If the target tissue 1412 is not in contact with the full length of the jaw 1402 only portions of the electrodes 1404, 1406 in proximity with the target tissue 1412 may be exposed. That is, portions of the electrodes 1404, 1406 not in contact with the target tissue 1412 or in close proximity to the target tissue 1412 may remain substantially covered by the insulators 1408, 1410. Accordingly, the insulators 1408, 1410 may reduce the likelihood of arcing associated with portions of the electrodes 1404, 1406 that are not in contact with the target tissue 1412.

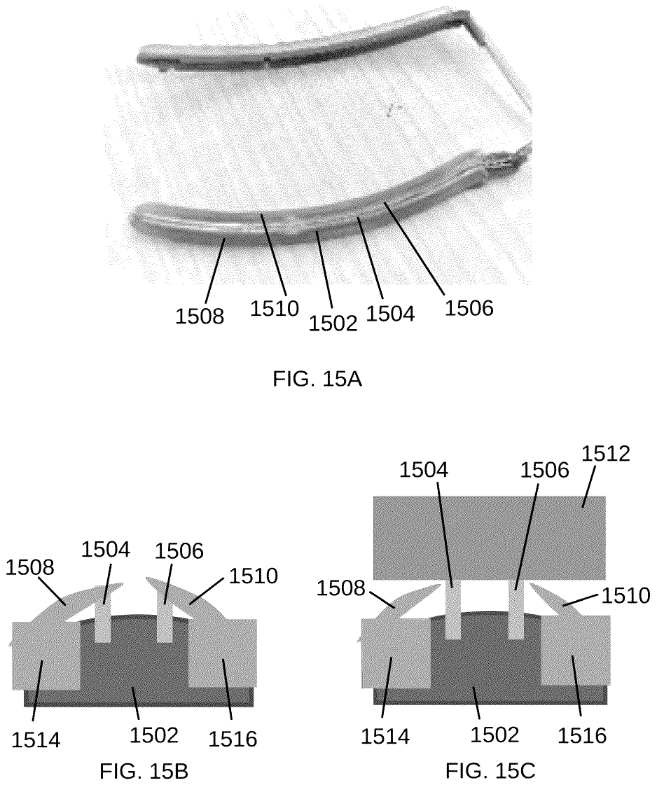

Referring to FIG. 15A, an alternative example configuration comprising a jaw with selectively insulated electrodes is illustrated. FIGS. 15B and 15C are section views of the embodiment of FIG. 15A with the electrodes in contact with the target tissue. In the illustrated embodiment, a jaw 1502 may include one or more electrodes 1504, 1506 on a tissue-engaging surface. Referring to FIG. 15B, one or more deformable insulators 1508, 1510 may at least partially cover the one or more electrodes 1504, 1506 when the jaw 1502 is not in contact with tissue. In some example embodiments, the insulators 1508, 1510 may be constructed from flexible silicone or other suitable materials. In the illustrated embodiment, each insulator 1508, 1510 substantially covers the entire length of a respective electrode 1504, 1506, with a central slit running along the length of the jaw 1502 between the inner aspects of the insulators 1508, 1510. Referring to FIG. 15C, when the jaw is placed into contact with the target tissue 1512, the target tissue 1512 may deform the insulators 1508, 1510 (e.g., generally laterally) to expose the electrodes 1504, 1506, thus allowing the target tissue 1512 to contact the electrodes 1504, 1506. If the target tissue extends substantially the full length of the jaw, substantially the full lengths of the electrodes 1504, 1506 may be exposed for contact with the target tissue 1512. If the target tissue 1512 is not in contact with the full length of the jaw 1502 only portions of the electrodes 1504, 1506 in proximity with the target tissue 1512 will be exposed. That is, portions of the electrodes 1504, 1506 not in contact with the target tissue 1512 or in close proximity to the target tissue 1512 may remain substantially covered by the insulators 1508, 1510. Accordingly, the insulators 1508, 1510 may reduce the likelihood of arcing associated with portions of the electrodes 1504, 1506 that are not in contact with the target tissue 1512. In the illustrated embodiments, the insulators 1508, 1510 of FIGS. 15A-15C differ from the insulators 1408, 1410 of FIGS. 14A-14C in that the insulators 1508, 1510 are disposed on respective, elongated base portions 1514, 1516, which may extend lengthwise along the lateral edges of the jaw 1502. In the illustrated embodiment, the insulators 1508, 1510 may be formed integrally with the respective base portions 1514, 1516. In some example embodiments, a main body of the jaw 1502 may be constructed from relatively rigid materials and/or the base portions 1514, 1516 and/or insulators 1508, 1510 may be constructed from relatively flexible, deformable materials.

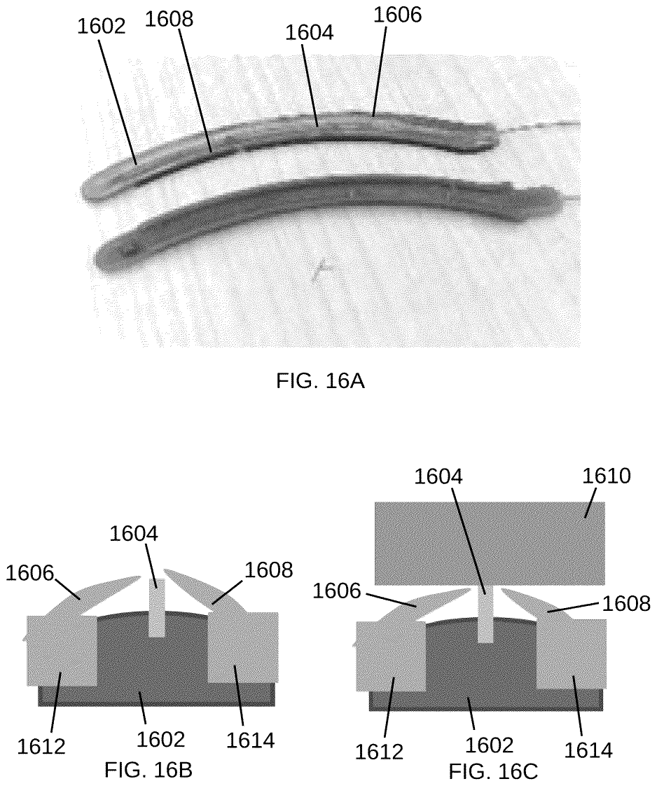

Referring to FIG. 16A, an alternative example configuration comprising a jaw with selectively insulated electrodes is illustrated. FIGS. 16B and 16C are section views of the embodiment of FIG. 16A with the electrode(s) in contact with the target tissue. In the illustrated embodiment, a jaw 1602 may include one or more electrodes 1604 on a tissue-engaging surface. Referring to FIG. 16B, one or more deformable insulators 1606, 1608 may at least partially cover the one or more electrodes 1604 when the jaw 1602 is not in contact with tissue. In some example embodiments, the insulators 1606, 1608 may be constructed from flexible silicone or other suitable materials, for example. In the illustrated embodiment, each insulator 1606, 1608 substantially covers the entire length of the electrode 1604 with a central slit running along the length of the jaw 1602 between the inner aspects of the insulators 1606, 1608. Referring to FIG. 16C, when the jaw is placed into contact with the target tissue 1610, the target tissue 1610 may deform the insulators 1606, 1608 (e.g., generally laterally) to expose the electrode 1604, thus allowing the target tissue 1610 to contact the electrode 1604. If the target tissue extends substantially the full length of the jaw, substantially the full length of the electrode 1604 may be exposed for contact with the target tissue 1610. If the target tissue 1610 is not in contact with the full length of the jaw 1602, only portions of the electrode 1604 in proximity with the target tissue 1610 may be exposed. That is, portions of the electrode 1604 not in contact with the target tissue 1610 or in close proximity to the target tissue 1610 may remain substantially covered by the insulators 1606, 1608. Accordingly, the insulators 1606, 1608 may reduce the likelihood of arcing associated with portions of the electrode 1604 that are not in contact with the target tissue 1610. In some example embodiments, the insulators 1606, 1608 may be disposed on respective, elongated base portions 1612, 1614, which may extend lengthwise along the lateral edges of the jaw 1602. In the illustrated embodiment, the insulators 1606, 1608 may be formed integrally with the respective base portions 1612, 1614. In some example embodiments, a main body of the jaw 1602 may be constructed from relatively rigid materials and/or the base portions 1612, 1614 and/or insulators 1606, 1608 may be constructed from relatively flexible, deformable materials. In the illustrated embodiments, the configuration of FIGS. 16A-16C may differ from the configuration of FIGS. 15A-15C in that it may include one elongated electrode 1604, rather than a pair of generally parallel, elongated electrodes 1504, 1506.

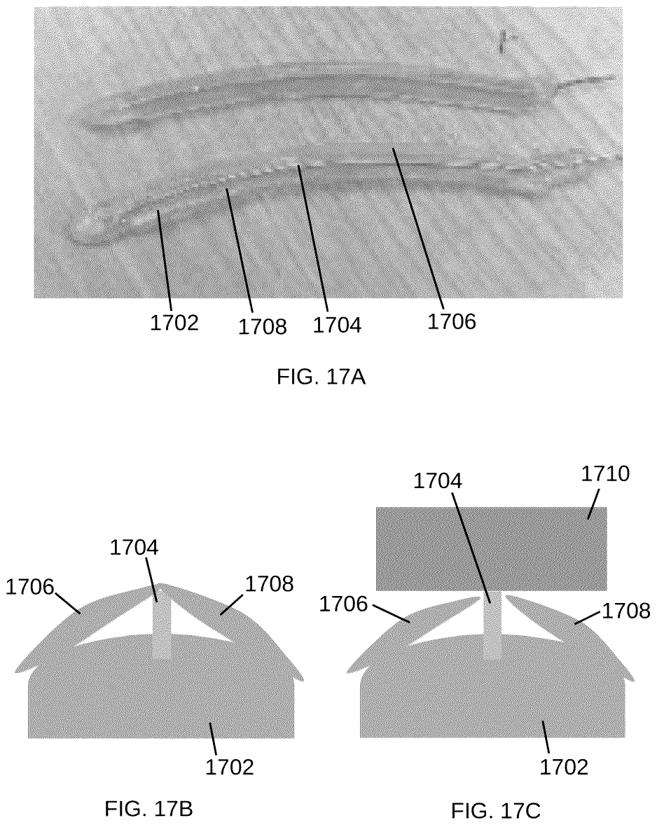

Referencing FIG. 17A, an alternative example configuration comprising a jaw with selectively insulated electrodes is illustrated. FIGS. 17B and 17C are section views of the embodiment of FIG. 17A with the electrodes in contact with the target tissue. In the illustrated embodiment, a jaw 1702 may include one or more electrodes 1704 on a tissue-engaging surface and one or more deformable insulators 1706, 1708, generally similar to the corresponding components described with reference to FIGS. 16A-16C. In the embodiment illustrated in FIGS. 17A-17C, a main body of the jaw 1702 may be formed integrally with the insulators 1706, 1708. In some embodiments, the main body of the jaw 1702 may be formed of the same material as the insulators 1706, 1708. Thus, in some embodiments, the main body of the jaw 1702 may be deformable. In some example embodiments, the main body of the jaw 1702 may be mounted to a rigid or more rigid jaw backer when assembled into a clamp-type configuration.

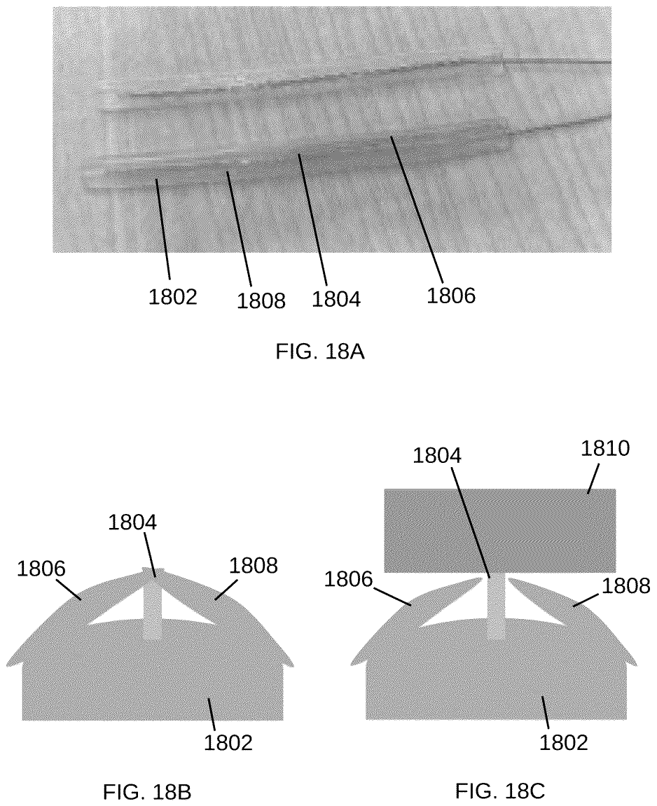

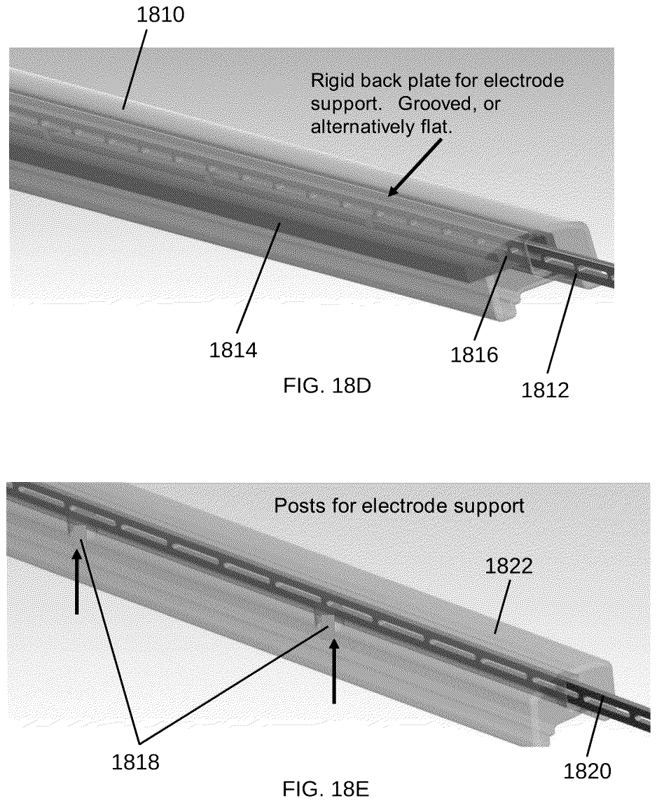

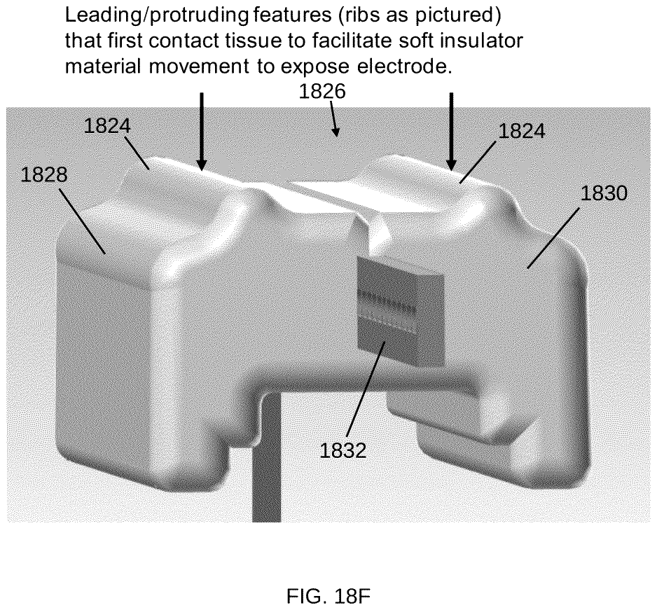

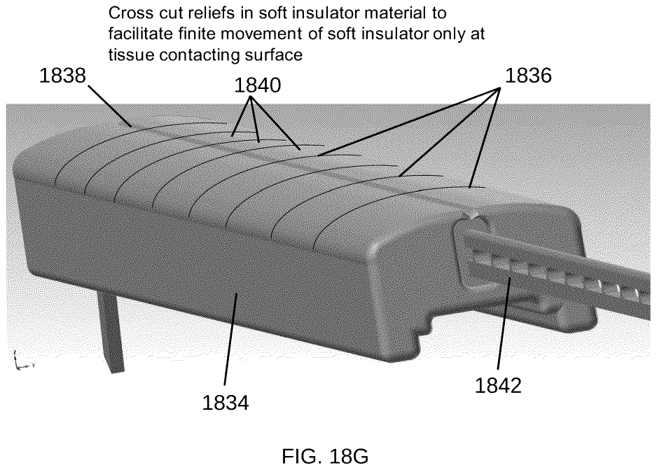

Turning to FIG. 18A, an alternative example configuration comprising a jaw with selectively insulated electrodes is illustrated. FIGS. 18B and 18C are section views of the embodiment of FIG. 18A with the electrodes in contact with the target tissue. In the illustrated embodiment, a jaw 1802 may include one or more electrodes 1804 on a tissue-engaging surface and one or more deformable insulators 1806, 1808, generally similar to the corresponding components described with reference to FIGS. 16A-16C. In the illustrated embodiment, the insulators 1806, 1808 may be at least partially overlapped, such as proximate the electrode 1804. In the illustrated embodiment, a main body of the jaw 1802 may be formed integrally with the insulators 1806, 1808. In some embodiments, the main body of the jaw 1802 may be formed of the same material as the insulators 1806, 1808. Thus, in some embodiments, the main body of the jaw 1802 may be deformable. In some example embodiments, the main body of the jaw 1802 may be mounted to a rigid or more rigid jaw backer when assembled into a clamp-type configuration. In the illustrated embodiments, the configuration of FIGS. 17A-17C also differs from the configuration of FIGS. 18A-18C in that jaw 1702 may be generally longitudinally curved, while jaw 1802 may be generally longitudinally straight.

FIG. 18D illustrates an example embodiment including a deformable insulator 1810 disposed about an electrode 1812 disposed on a rigid back plate 1814. In the illustrated embodiment, the back plate may include one or more longitudinal grooves 1816, such as to receive the electrode therein. In some alternative embodiments, the back plate 1814 may be generally flat (e.g., without grooves).

FIG. 18E illustrates an example embodiment including spaced-apart posts 1818 supporting an electrode 1820 within a deformable insulator 1822. The posts 1818 may be mechanically coupled to a relatively rigid portion support element, such a jaw structure of a clamp-type PFA device. In some embodiments, the insulator 1822 may include one or more openings configured to receive the posts 1818 therethrough.

Some example embodiments may include one or more leading, raised features configured to facilitate movement of insulator material, such as to expose an electrode. For example, as illustrated in FIG. 18F, one or more raised features 1824 on a tissue-contacting surface 1826. These features may be arranged to contact a target tissue before other portions of the insulators 1828, 1830, thus causing the insulators 1828, 1830 to move away from and exposing the electrode 1832.

FIG. 18G illustrates an alternative example embodiment in which a deformable insulator 1834 may be segmented. In the illustrated embodiment, one or more generally lateral cross cuts 1836 (e.g., slits) are provided in addition to a longitudinal slit 1838. Accordingly, segments 1840 of the insulator 1834 in contact with tissue may more easily move away from the electrode 1842, while segments 1840 of the insulator 1834 not in contact with tissue may remain in place (e.g., at least partially covering the electrode 1842).

The present disclosure contemplates that microbubbles may be formed when high-voltage electrical pulses are delivered during PFA. Generally, microbubbles may include a plurality of thin spheres of liquid respectively encapsulating a small pocket of gas. Microbubbles may be formed by liquid vaporization, cavitation, and/or electrolysis, for example. Generally, microbubble formation may be undesirable because, after being formed, the microbubbles may travel through the bloodstream and block small blood vessels, potentially leading to unintended tissue damage and/or organ dysfunction. Microbubbles may also cause silent cerebral events, such as brain injuries that occur during a medical procedure or intervention without producing any noticeable symptoms.

The present disclosure contemplates that although PFA is generally considered non-thermal in that it does not rely on high temperatures to ablate tissue, in some circumstances, application of PFA energy may result in tissue heating. Generally, the duration, intensity, and/or frequency of the PFA signal may affect the extent of tissue heating. In some circumstances, the composition and/or structure of the tissue may affect heating, such as how quickly heat may be transmitted and dissipated. In some circumstances, cooling methods may be utilized to reduce tissue temperature. For example, irrigation with a cooling fluid such as saline and/or use of a heat sink or cooling catheter may reduce the likelihood of undesired heating. Alternatively, in vacuum pod embodiments, fluid flow through the vacuum pod may be employed to cool the electrode and tissue surface. Electrode pair alternating switching may be employed for on/off electrode duty cycling.

The present disclosure contemplates that PFA and RF ablation may be associated with different mechanisms of action and/or potentially different advantages and/or disadvantages. In some example embodiments according to at least some aspects of the present disclosure, these differences may be utilized to facilitate a desired outcome. For example, some illustrative embodiments may be configured to conduct both PFA and RF ablation on a particular target tissue. In some example embodiments, PFA and RF ablation may be performed using at least one electrode in common. In some example embodiments PFA and RF ablation may be performed using different electrodes. In general, any system, device, electrode, insulation configuration, etc., described herein may be used in connection with delivery of either or both RF and PFA.

For example, some embodiments may be user-selectable between PFA-only and RF-only modes. Accordingly, a user may select a desired ablation modality for a particular ablation. For example, a surgeon may select PFA and/or RF ablation based at least in part upon the location of the ablation (e.g., proximity to sensitive, non-target tissues) and/or the target tissue type.

Some example embodiments may be configured to create lesions using both PFA and RF ablation modalities. For example, in some circumstances, it may be advantageous to perform a PFA endocardially in connection with an epicardial RF ablation. The resulting ablation may be partially PFA and partially RF at any mixed ratio meeting within the tissue thickness to create a full thickness lesion. PFA may extend from one surface into the tissue thickness while RF extends from the opposite surface. Alternatively, the RF lesion may partially or wholly be formed centrally within the thickness of the tissue with PF completing the lesion outward to the tissue surfaces. Such a mixed modality approach may create a transmural lesion in the target tissue while benefiting from advantages associated with each individual modality. For example, in some circumstances, using PFA endocardially may avoid some potential disadvantages of using RF ablation in close proximity to blood and/or using RF ablation epicardially may facilitate thermal ablation of some targeted autonomic nervous tissues. In some example embodiments, PFA may be applied bidirectionally (e.g., endocardially and epicardially), and RF ablation may be applied unidirectionally (e.g., epicardially only). In some example embodiments, both PFA and RF ablation may be conducted substantially across a full tissue thickness.

In some example embodiments, PFA may be conducted before RF ablation, which may facilitate faster RF ablation due to the increased tissue conductivity caused by the PFA. In some example embodiments, RF ablation may be conducted before PFA. In some example embodiments, the RF and PF ablations may be performed simultaneously with interrupted alternating delivery and/or overlapping delivery.

The present disclosure contemplates that PFA and cryoablation may be associated with different mechanisms of action and/or different advantages and disadvantages. In some example embodiments according to at least some aspects of the present disclosure, these differences may be utilized to facilitate a desired outcome. For example, some illustrative embodiments may be configured to conduct both PFA and cryoablation, not necessarily at the same time.

For example, some embodiments may be user-selectable between PFA-only and cryoablation-only modes. Accordingly, a user may select a desired ablation modality for a particular ablation. For example, a surgeon may select PFA and/or cryoablation based at least in part upon the location of the ablation and/or the target tissue type.

Some example embodiments may be configured to create lesions using both PFA and cryoablation modalities. Such a mixed modality approach may create a transmural lesion in the target tissue while benefiting from advantages associated with each individual modality. In some example embodiments, PFA may be conducted before cryoablation. In some example embodiments, cryoablation may be conducted before PFA. In some example embodiments, PFA may be conducted during cryoablation at any point in the cryo delivery or for the entire duration. A temperature measurement or setpoint may or may not be employed as feedback. In some example embodiments, the cryoablation may be applied at a therapeutic level. That is, the cryoablation, itself, may be sufficient to cause permanent lesion formation in the target tissue. In other embodiments, the cryoablation may be applied at a sub-therapeutic level. That is, the cryoablation, itself, may affect the tissue in a substantially reversible manner. In some circumstances, performing cryoablation before PFA may affect (e.g., improve or enhance) the subsequent PFA. For example, cryo-treated target tissue either fully rewarmed or still cooled below body temperature may more efficiently receive PFA energy, or the PFA energy may be conducted through the target tissue differently, or the lower tissue temperature effectuated using cryoablation may offset or reduce the thermal temperature increase effectuated from using PFA.

The present disclosure contemplates that, in some circumstances, a PFA-created lesion may not be readily visible on the target tissue immediately or shortly after delivery of the PFA energy. In some cases, a PFA lesion may not be readily visible or detectable for days to weeks, as cell death and tissue response occurs. Accordingly, the existence, location, and/or extent of a PFA lesion may not be readily apparent to a user or detectable during an ablation procedure. The present disclosure contemplates that this lack of immediate visibility and/or detectability may increase the difficulty of creating elongated, continuous lesions formed by conducting multiple, overlapping ablations.

Turning back to FIG. 1, in some example embodiments according to at least some aspects of the present disclosure, a PFA device 104 may be operated to create an immediately visible, thermal lesion in connection with creating a PFA lesion. For example, a PFA lesion may be formed and then a corresponding thermal lesion may be formed using RF ablation and/or cryoablation (e.g., without moving the end effector 132 between the PFA and RF or cryo ablation). Alternatively, high-voltage PFA may be utilized to create a localized thermal lesion in addition to ablating the surrounding tissue via irreversible electroporation. Alternatively, PFA may be conducted, followed by a high-voltage pulse or pulse train that may not electrically porate the tissue but may create a thermal lesion in the tissue. In some example embodiments, a thermally induced lesion may be advantageous as it may ablate surface plexuses.

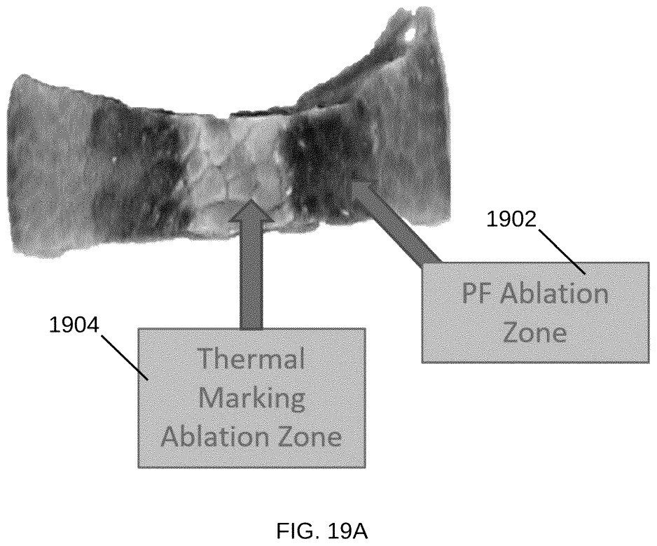

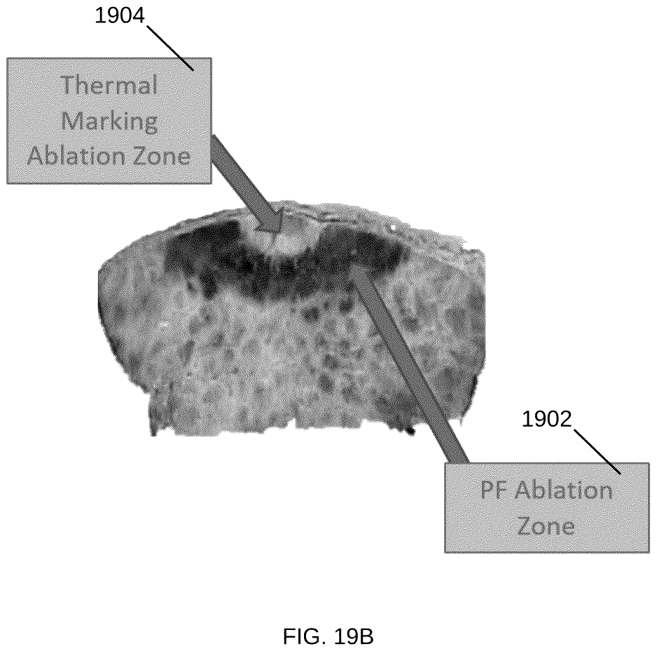

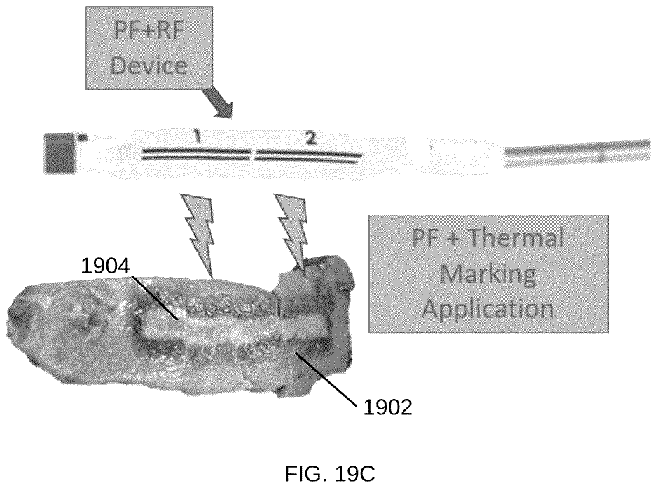

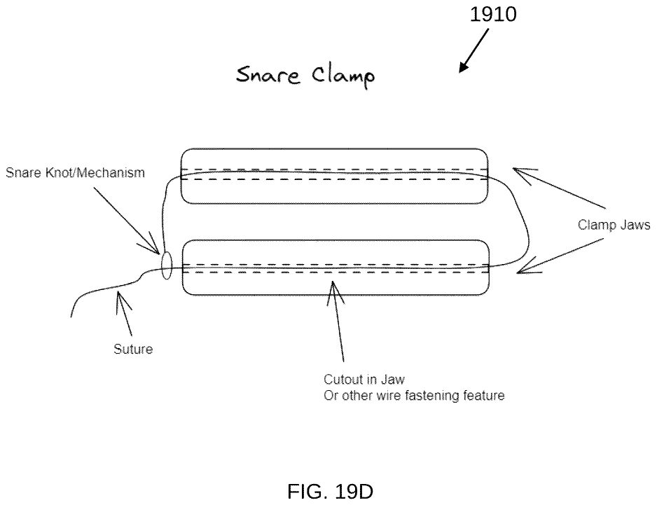

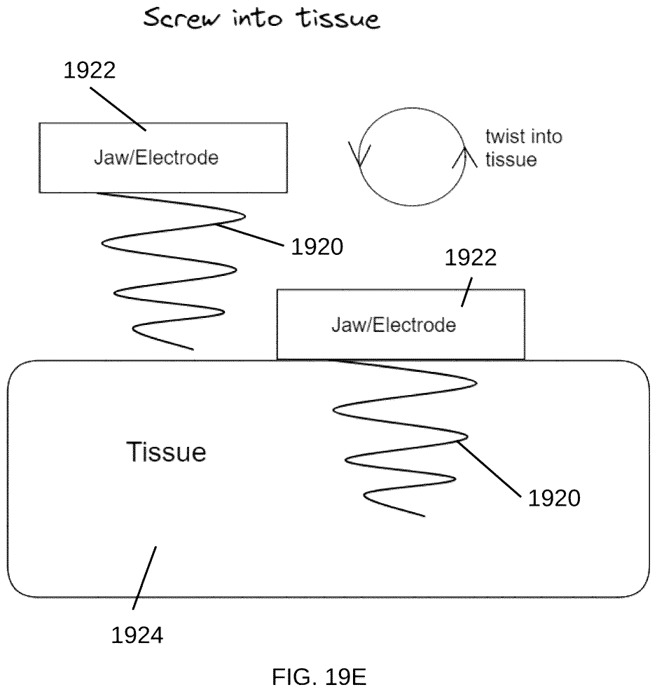

Referencing FIG. 19A, an example lesion including a PFA zone 1902 and a thermal ablation zone 1904 is depicted. FIG. 19B is a section view of the lesion of FIG. 19A.

FIG. 19C is a top view of an example lesion created using a PFA+RF device, as shown having a PFA zone 1902 and a thermal ablation zone 1904, according to at least some aspects of the present disclosure.

Example embodiments according to at least some aspects of the present disclosure may utilize various tissue contact configurations.

Some example embodiments may be configured to utilize operator-applied contact forces to cause the desired tissue contact. For example, pen-type configurations may be manually held against the target tissue by an operator during application of PFA energy. Similarly, a surgical robot may be used to apply a PFA device against a target tissue.

Some embodiments utilizing mechanical configurations may be constructed generally in the form of a clamp. See, for example, the embodiment of FIG. 2. Some embodiments utilizing mechanical configurations may utilize a snare. FIG. 19D illustrates an example snare clamp 1910.

Some example embodiments may utilize a screw for tissue engagement. FIG. 19E illustrates an example generally helical screw engagement element 1920 for a jaw or electrode 1922 configured to penetrate the target tissue 1924.

Some example embodiments may utilize cooperating magnetic elements for tissue engagement. For example, various embodiments described in International Application No. PCT/US2022/082057, filed Dec. 20, 2022, published as International Publication No. WO2023129842 on Jul. 6, 2023, titled “MAGNETICALLY COUPLED ABLATION COMPONENTS”, which is incorporated herein by reference, may be utilized in connection with embodiments according to at least some aspects of the present disclosure.

Some example embodiments may be configured to pierce tissue. See, for example, the needle-type embodiments of FIGS. 4A-4C.

Some example embodiments may be configured to utilize vacuum for tissue engagement. See, for example, the embodiments of FIGS. 3A and 3B.

Some example embodiments may utilize tissue freezing for tissue engagement. For example, an embodiment including cryogenic capabilities may be placed into contact with a target tissue. The tissue may be at least partially frozen or significantly cooled, therapeutically or sub-therapeutically, which may cause a tissue engaging probe to adhere to the target tissue. While the probe is adhered to the target tissue, thus maintaining contact, PFA energy may be applied. After the desired PFA and/or cryogenic effects have been achieved, the tissue may be thawed or heated and the probe may be removed from the tissue.

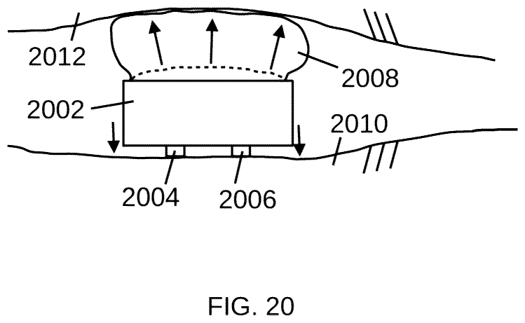

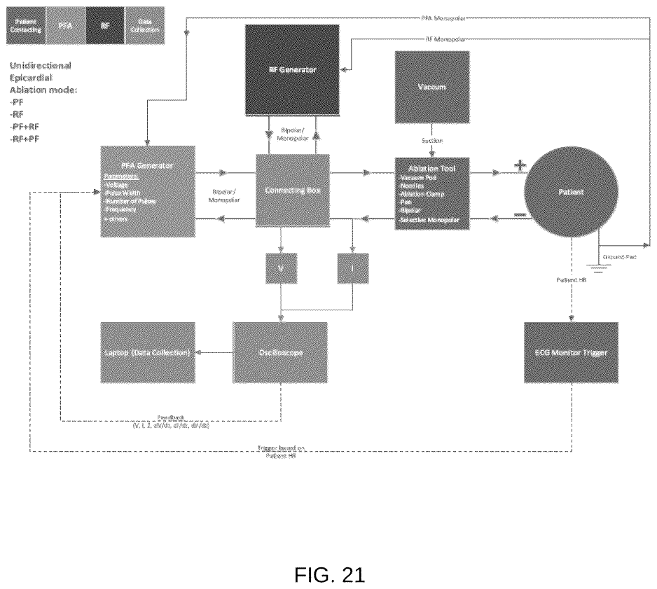

Referring to FIG. 20, some example embodiments may utilize one or more expandable structures to generate a tissue contacting force. In the illustrated embodiment, a PFA device 2002 may include one or more electrodes 2004, 2006 on a tissue-engaging surface and/or one or more expandable structures 2008. In the illustrated embodiment, the expandable structure 2008 may be disposed generally opposite the electrodes 2004, 2006. In operation, the PFA device 2002 may be positioned between a target tissue 2010 and an opposed tissue 2012. The expandable structure 2008 may be expanded from a collapsed configuration (dashed line) to an expanded configuration (solid line). Expanding the expandable structure 2008 may cause it to contact the opposed tissue 2012, thereby pressing the tissue-engaging surface comprising the electrodes 2004, 2006 against the target tissue 2010. Expansion of the expandable structure 2008 may be controlled as necessary to achieve the desired contact between the electrodes 2004, 2006 and the target tissue 2010. Upon completion of one or more ablations, the expandable structure 2008 may be collapsed. In some example embodiments, the expandable structure may be inflatable, such as by a fluid (e.g., gas and/or liquid). In an example embodiment, the target tissue 2010 may comprise a myocardium and the opposed tissue 2012 may comprise a pericardium.

Some example embodiments according to at least some aspects of the present disclosure may include a plurality of electrodes. Generally, any embodiment described herein may be provided with one or multiple electrodes, unless explicitly stated otherwise. While some embodiments may have been described in connection with particular exemplary uses of particular individual electrodes, it is to be understood that any electrode on any embodiment may be used for any purpose, regardless of how it may be described in a specific example. For example, an electrode described as an ablation electrode may be used for pacing, stimulating, mapping, and/or sensing in some circumstances. Similarly, an electrode described as a pacing, stimulating, mapping, and/or sensing electrode may be used for ablation in certain circumstances. Further, whether or not specifically described herein in connection with a particular example embodiment, it is to be understood that any embodiment according to at least some aspects of the present disclosure may include additional electrodes, such as for pacing, stimulating, mapping, and/or sensing.

Some example embodiments according to at least some aspects of the present disclosure may be used in connection with procedures directed to treatment of various arrythmias. For example, ablations may be performed on various target tissues comprising the cardiac autonomic nervous system (e.g., ganglionated plexuses, nodes, and/or conduction pathways) and/or cardiac substrate tissues (e.g., atria and/or ventricles).

Generally, it is within the scope of this disclosure to conduct procedures involving any portions of the heart using apparatus and/or methods disclosed herein. For example, procedures involving the right atrium may be performed in connection with treatment for inappropriate sinus tachycardia (e.g., crista line, inferior vena cava, and/or superior vena cava), atrial fibrillation (e.g., Cox maze lesions—right side), and/or Wolff-Parkinson-White Syndrome. Procedures involving the right ventricle may be performed in connection with treatment for ventricular tachycardia (e.g., right ventricle posterior wall, right ventricle lateral free wall, right ventricle anterior, septum, right ventricle papillary muscles, and/or right ventricle outflow tract), partial ventricular contractors (e.g., right ventricle outflow tract septum, basal right ventricle, and/or right ventricle outflow tract free wall), and/or Brugada Syndrome (e.g., right ventricle outflow tract), for example. Procedures involving the left atrium may be performed in connection with treatment for atrial fibrillation (e.g., ligament of Marshall, roof and floor lines, left atrium posterior wall, isthmus line, and/or autonomics (ganglionated plexus)) and/or left atrial appendage isolation (e.g., left atrial appendage ostium). Procedures involving the left ventricle may be performed in connection with syncope (e.g., autonomics (ganglionated plexus)), atrial tachycardia (e.g., anywhere in the left ventricle), atrial flutter (e.g., mitral valve), Wolff-Parkinson-White Syndrome (e.g., atrioventricular groove), partial ventricular contractions (e.g., left ventricle outflow tract and/or aortic root), hypertension (e.g., anywhere in the left ventricle), and/or ventricular tachycardia (e.g., left ventricle posterior wall, left ventricle lateral free wall, left ventricle anterior, septum, left ventricle papillary muscles, and/or left ventricle summit), for example. Procedures involving the right ventricle/left ventricle septum may be performed in connection with ventricular tachycardia (e.g., combined right ventricle and left ventricle lesion), for example. It will be understood that the foregoing list is merely exemplary and is not to be considered limiting.

Some example embodiments according to at least some aspects of the present disclosure may be used in connection with nerve block procedures. For example, peripheral nerves may be ablated to create a temporary yet fully recoverable loss of sensory nerve function. Ablation may cause axonotmesis, a level of nerve injury according to Seddon's classification in which the axons and the myelin are disrupted but at least some of the surrounding tubular structures, such as the endoneurium, perineurium, fascicle, and/or epineurium, remain intact. The ensuing Wallerian degeneration, a process in which the entire length of the nerve segment distal to the ablation lesion is dismantled, may take approximately 1 week. Regeneration of the nerve begins from the proximal segment and continues at an average rate of 1-3 mm/day, following the intact structural components until the tissue is reinnervated. This process can take weeks to months depending on how significant the ablation lesion is on the tissue. Because it preserves the structure of the nerve, such procedures may not be associated with development of neuromas.

Local analgesia to a nerve (e.g., intercostal nerve) is intended for managing pain due to incision, surgical muscle disruption, discomfort from nerve impingement by the surgical equipment (e.g., retractors) and surgical retainers (e.g., sutures), and for any opening created by a tube or trocar site. In exemplary form, one exemplary process comprises nerve ablation for post-thoracotomy pain that includes ablation of the intercostal nerves. What follows is an exemplary procedure for conducting a nerve block responsive to a thoracotomy that is effective for pain management and may be applied to any nerve within an animal body.

It may be recommended to perform the nerve ablation procedure as early as possible in the surgical procedure, such as prior to or immediately following creation of the thoracotomy. The target nerve, such as an intercostal nerve, may be located in the incisional intercostal space (e.g., between the ribs), preferably at the margin of the innermost intercostal muscle and the membranous portion of the internal intercostal muscle. A location may be chosen that is proximal to the lateral cutaneous branch but at least 2 cm from the ganglia and/or at least 4 cm from the spine.

The ablation device may be placed directly on top of the nerve, optionally with a slight angulation that assures the nerve is directly under the ablation element. Prior to ablation, the ablation device may be pressed into the costal groove with enough pressure to create compression of the tissue for stability and reduced local perfusion. Adequate pressure may be pressure sufficient to create blanching if depressed against the skin. In some example embodiments, a needle-type PFA device may be used. In some example embodiments, a pen-type PFA device may be used. In some example embodiments, a minimally invasive PFA device may be used, such as one providing vacuum stabilization capability.

Post locating the ablation device to contact the nerve or in close proximity thereto, the ablation device may be activated to ablate the nerve. The ablation sequence may be repeated at another location of the same nerve (or at a different location of a different nerve) and repeated as necessary to achieve the proper pain management result. In general, some exemplary nerve ablation procedures as described above may be repeated on the intercostal nerves located in each of the third to ninth intercostal spaces.

In some example methods according to at least some aspects of the present disclosure, nerve ablation may be provided in connection with amputation of a limb and/or an extremity. The present disclosure contemplates that, in some circumstances, nerve ablation may be performed at some time after an amputation procedure is performed (e.g., weeks, months, or years), such as after the occurrence of significant pain for the patient.

In some example methods according to at least some aspects of the present disclosure, nerve ablation may be performed concomitant with the amputation procedure. For example, during an amputation procedure, a nerve may be identified. The nerve may be dissected, separating it from adjacent tissues, such as nearby blood vessels. A nerve section location may be determined. In some cases, the nerve may be retracted distally. A nerve ablation location may be determined, such as proximal to the nerve transection location. The nerve may be engaged at the ablation location, such as using an ablation device. Ablation of the nerve may be performed using the ablation device either by contact with a pen like device or capturing between a clamp like or grasper like device. In some cases, one or more ablation cycles may be performed. The ablation device may be removed from the nerve. The nerve may be sectioned at the nerve section location.

Thus, the mechanical injury to the nerve (e.g., the transection of the nerve at the nerve section location) may be some distance distal to the nerve ablation location. Because the nerve may slowly regenerate distally from the ablation location towards the nerve section location, it may take substantial time until the regenerated nerve reaches the nerve section location. During this time, injured tissues proximate the nerve section location may heal. As a result, when the regenerated nerve reaches the nerve section location, the nerve may be surrounded by relatively healed tissue, thus reducing the likelihood and/or severity of neuroma formation. Additionally, during the time it takes for the nerve to regenerate, pain and other sensations from locations distal to the ablation location may be reduced, thus reducing the need for other post-operative pain management.

Some example embodiments according to at least some aspects of the present disclosure may be utilized in connection with ablation of target tissues other than cardiac tissue and nervous tissue. For example, some embodiments may be used in connection with ablation of tissues including liver tissue, kidney tissue, and/or brain tissue.

Turning to FIG. 21, a simplified block diagram of an example equipment configuration, which may be used, for example, for using various PFA and/or RF ablation devices and/or algorithms according to at least some aspects of the present disclosure is illustrated.

Referencing FIG. 22, an example PFA device 2200 may be configured for vacuum-stabilization and/or monopolar energy delivery. The illustrated embodiment may be constructed and/or operated generally similar to those described in U.S. Pat. No. 10,413,355, issued Sep. 17, 2019, titled “VACUUM COAGULATION PROBES,” which is incorporated by reference herein. Referring to FIGS. 5R and 22, some example embodiments may include a generally helical electrode 2202 disposed within a vacuum pod, which may engage the target tissue using vacuum and/or which may be supplied with saline solution, which may facilitate cooling and/or coupling).

Referring to FIG. 23, in some example embodiments according to at least some aspects of the present disclosure, one or more PFA energy parameters, such as those described herein, may be varied based at least in part upon one or more measured parameters. For example, prior to delivering PFA energy, one or more measurements may be conducted, and such measured values may be used to determine one or more PFA energy parameters that will be delivered. In some example embodiments, one or more measurements may be conducted in connection with delivery of PFA energy (e.g., during and/or between PFA pulses), and such measured values may be used to determine whether to continue or stop PFA energy delivery and/or may be used to determine whether to adjust one or more PFA energy parameters.

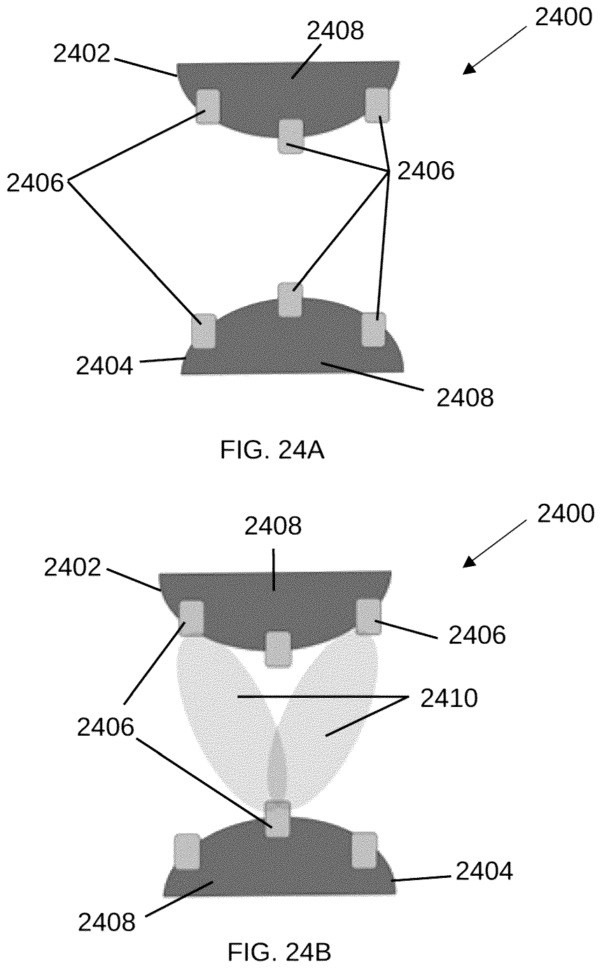

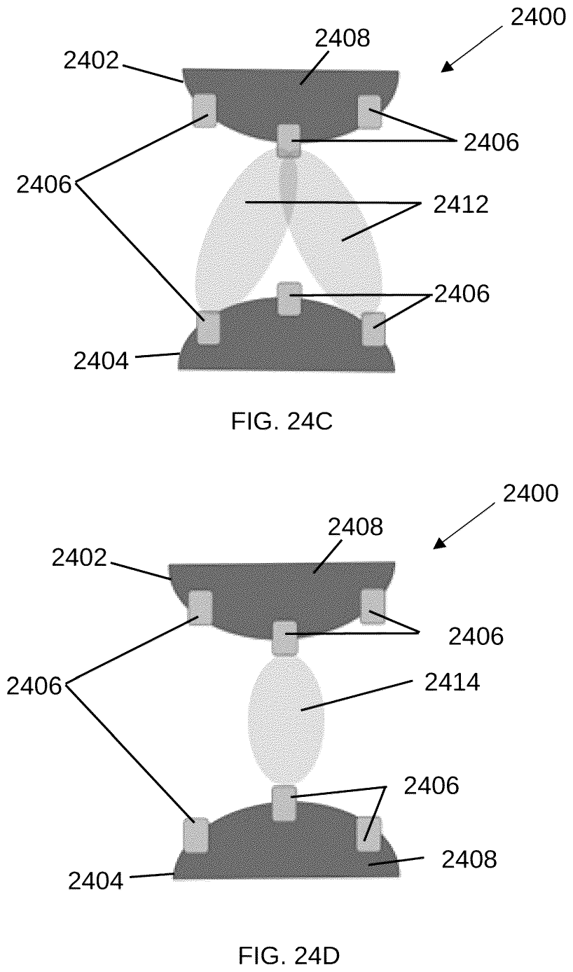

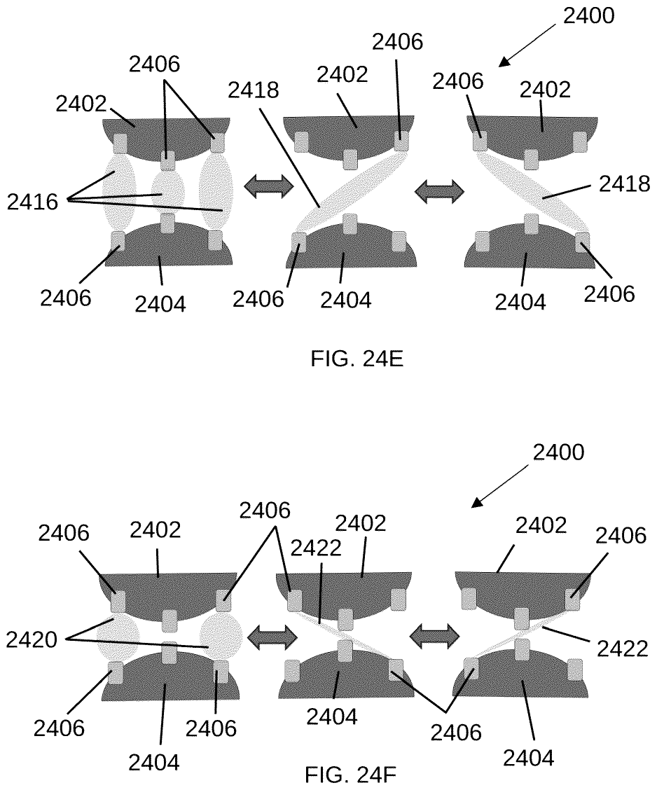

Referring to FIGS. 24A-24D, in some example embodiments according to at least some aspects of the present disclosure, a clamp style electrode configuration 2400 may include a first jaw 2402 opposite a second jaw 2404, where the jaws are selectively opened and closed to vary the distance between electrodes 2406 on the jaws. In exemplary form, the electrodes 2406 may be embedded in an insulating substrate 2408 and include associated leads (not shown) in order to provide electrical communication with a PFA device (such as PFA device 104 as disclosed herein). By way of further example, the electrodes 2406 may extend along substantially the entire longitudinal length of the jaws 2402, 2404 or may extend for some distance shorter than the longitudinal length of the jaws. It should also be understood that the electrodes 2406 may comprise any number of segments distributed longitudinally. Moreover, the electrodes 2406 may have uniform widths, or may have widths that vary depending upon the position of the electrodes. For example, the widths of electrodes 2406 on the outer periphery may be wider than the widths of the electrodes toward the longitudinal center of the jaws 2402, 2404. Conversely, the widths of electrodes 2406 on the outer periphery may be narrower than the widths of the electrodes toward the longitudinal center of the jaws 2402, 2404. Moreover, the heights of electrodes 2406 extending above the insulating substrate 2408 on the outer periphery may be greater than the heights of the electrodes toward the longitudinal center of the jaws 2402, 2404. Further, the shape of electrodes 2406 extending above the insulating substrate 2408 may embody a rectangular cross-section along the longitudinal dimension, through various shapes of electrodes may be utilized. Still further, the shape of the electrodes 2406 (such as having rounded or bulbous longitudinal cross-section) on the outer periphery may be different from the shape of the electrodes (such as having a rectangular longitudinal cross-section) toward the longitudinal center of the jaws 2402, 2404. It is to be understood that the jaws 2402, 2404 may be used in lieu of the jaws 210, 212 as discussed herein and depicted in FIG. 2 for the exemplary PFA device 200 as part of clamping tissue between the jaws.

In a first exemplary configuration, the insulating substrate 2408 of each jaw 2402, 2404 may exhibit a convex profile (longitudinal cross-section) that may be semi-spherical or have a bulbous shape so that at least one electrode 2406 extends outward from the insulating substrate. Having jaws 2402, 2404 with convex profiles that face one another (as shown in FIG. 24A) may be advantageous in order to push excess fluid out of tissue clamped between the jaws. By reducing the fluid content of tissue clamped between the jaws 2402, 2404, the probability of steam pops and tissue perforation is reduced when the electrodes are operated in a radio frequency (RF) mode.

In exemplary form, each jaw 2402, 2404 may include one or more electrodes 2406 (one, two, three, four, five, etc.), with multiple electrodes being spaced apart from one another with respect to an outer perimeter across the convex profile. By way of example, FIGS. 24A-24D exhibits a pair of jaws 2402, 2404, with each jaw including three electrodes 2406. It has been determined that as the distance(s)/gap(s) between corresponding electrodes 2406 increases, the critical electric field for PFA extends further laterally from the jaws 2402, 2404. In other words, as the distance(s)/gap(s) between corresponding electrodes 2406 increases, the width of a corresponding PFA lesion will increase. In certain instances, increased PFA lesion width may be undesirable because it limits the position of the PFA device, particularly in proximity to critical anatomical structures. Simply put, a surgeon should provide a sufficient lateral margin between the jaws 2402, 2404 and critical anatomical structures to avoid ablating these critical structures. The instant inventors have determined that application of PFA can still be safe and less sensitive to critical anatomical structures by making use of multiplexing.

Looking specifically at FIGS. 24B-24D, jaws 2402, 2404 incorporating multiple electrodes 2406 allow one to multiplex pulses across multiple electrodes as part of PFA. For example, as depicted in FIG. 24B, outer electrodes 2406 of the first jaw 2402 may be paired with a central electrode of the second jaw 2404, which is operative to generate elongated energy fields 2410 that do not exhibit excess lateral creep and correspondingly constrain the area to be ablated as being between the jaw electrodes 2406. PFA may be preformed for a predetermined period using this first electrode multiplexing scheme. Alternatively, or in addition, as depicted in FIG. 24C, outer electrodes 2406 of the second jaw 2404 may be paired with the central electrode of the first jaw 2402, which is operative to generate elongated energy fields 2412 that do not exhibit excess lateral creep and correspondingly constrain the area to be ablated as being between the jaw electrodes 2406. PFA may be preformed for a predetermined period using this second electrode multiplexing scheme. Alternatively, or in addition to the first and/or second multiplexing scheme, as depicted in FIG. 24D, central electrode 2406 of the first jaw 2402 may be paired with the central electrode of the second jaw 2404, which is operative to generate an elongated energy fields 2414 that does not exhibit excess lateral creep and correspondingly constrains the area to be ablated as being between the jaw electrodes 2406. In accordance with the instant disclosure, the one or more of the foregoing multiplexing schemes may be carried out sequentially or simultaneously.

Turning to FIG. 24E, in a circumstance where the distance(s)/gap(s) between corresponding electrodes 2406 of the jaws 2402, 2404 is less than depicted in FIGS. 24A-24D, indicative of the jaws clamping moderately thinner tissue, a further alternative electrode scheme may also be utilized in lieu of the foregoing schemes or in addition to the foregoing schemes. By way of example, this alternate exemplary scheme may include conducting PFA vertically across counterpart electrodes 2406 of the jaws 2402, 2404 simultaneously or consecutively to create the energy fields 2416, with the number of elongated energy fields, optionally corresponding to the paired number of electrodes. This same scheme may include conducting PFA diagonally (vertically offset) across counterpart electrodes 2406 of the jaws 2402, 2404 simultaneously or consecutively to create the elongated energy fields 2418, with the number of elongated energy fields, optionally corresponding to the paired number of electrodes.

Turning to FIG. 24F, in a circumstance where the distance(s)/gap(s) between corresponding electrodes 2406 of the jaws 2402, 2404 is less than depicted in FIG. 24E, indicative of the jaws clamping even thinner tissue, a still further alternative electrode scheme may also be utilized in lieu of the foregoing schemes or in addition to the foregoing schemes. By way of example, this further alternate exemplary scheme may include conducting PFA vertically across counterpart electrodes 2406 of the jaws 2402, 2404 simultaneously or consecutively to create the energy fields 2420. This same scheme may include conducting PFA diagonally (vertically offset) across counterpart electrodes 2406 of the jaws 2402, 2404 simultaneously or consecutively to create the elongated energy fields 2422, with the number of elongated energy fields, optionally corresponding to the paired number of electrodes.

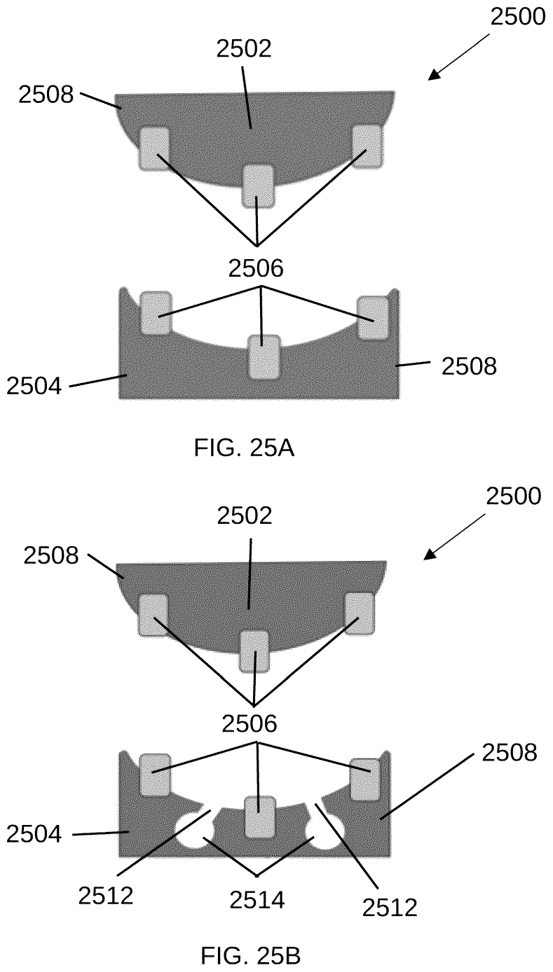

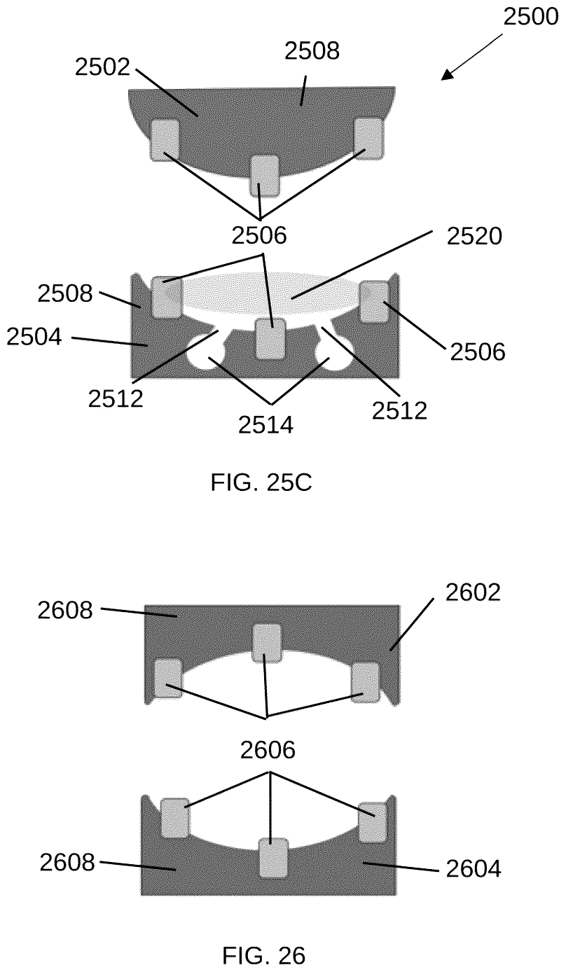

Referring to FIGS. 25A-25C, in some example embodiments according to at least some aspects of the present disclosure, a clamp style electrode configuration 2500 may include a first jaw 2502 opposite a second jaw 2504, where the jaws are selectively opened and closed to vary the distance between electrodes 2506 on the jaws. In exemplary form, the electrodes 2506 may be embedded in an insulating substrate 2508 and include associated leads (not shown) in order to provide electrical communication with a PFA device (such as PFA device 104 as disclosed herein). By way of further example, the electrodes 2506 may extend along substantially the entire longitudinal length of the jaws 2502, 2504 or may extend for some distance shorter than the longitudinal length of the jaws. It should also be understood that the electrodes 2506 may comprise any number of segments distributed longitudinally. Moreover, the electrodes 2506 may have uniform widths, or may have widths that vary depending upon the position of the electrodes. For example, the widths of electrodes 2506 on the outer periphery may be wider than the widths of the electrodes toward the longitudinal center of the jaws 2502, 2504. Conversely, the widths of electrodes 2506 on the outer periphery may be narrower than the widths of the electrodes toward the longitudinal center of the jaws 2502, 2504. Moreover, the heights of electrodes 2506 extending above the insulating substrate 2508 on the outer periphery may be greater than the heights of the electrodes toward the longitudinal center of the jaws 2502, 2504. Further, the shape of electrodes 2506 extending above the insulating substrate 2508 may embody a rectangular cross-section along the longitudinal dimension, through various shapes of electrodes may be utilized. Still further, the shape of the electrodes 2506 (such as having rounded or bulbous longitudinal cross-section) on the outer periphery may be different from the shape of the electrodes (such as having a rectangular longitudinal cross-section) toward the longitudinal center of the jaws 2502, 2504. It is to be understood that the jaws 2502, 2504 may be used in lieu of the jaws 210, 212 as discussed herein and depicted in FIG. 2 for the exemplary PFA device 200 as part of clamping tissue between the jaws.

In a first exemplary configuration, the insulating substrate 2508 of the first jaw 2502 may exhibit a convex profile (longitudinal cross-section) that may be semi-spherical or have a bulbous shape so that at least one electrode 2506 extends outward from the insulating substrate. And the insulating substrate 2508 of the second jaw 2504 may exhibit a concave profile (longitudinal cross-section) that may be arcuate so that at least one electrode 2506 extends outward from the arcuate shaped insulating substrate. In this fashion, the jaws 2502, 2504 may have complementary longitudinal profiles that may facilitate nesting of one jaw 2502 with respect to the other jaw 2504 when clamping tissue therebetween. These complementary shaped jaws 2502, 2504 may be advantageous to align the jaws laterally with respect to one another when tissue is clamped therebetween so that the longitudinal aspects of the jaws correctly align and overlap one another. Moreover, having complementary shaped jaws 2502, 2504 (one convex, one concave) may be advantageous to allow for lateral pressure gradients between the tissue and jaws. More specifically, to the extent that the jaws 2502, 2504 exhibit complementary concave and convex insulating substrates 2508, it may be advantageous for the radius of curvature of the convex profile to be less than the radius of curvature of the concave profile. Alternatively, the jaws 2502, 2504 may exhibit complementary concave and convex insulating substrates 2508 having an identical or nearly identical radius of curvature of the convex profile and of the concave profile as a means to provide lesser or no lateral pressure gradients (i.e., uniform lateral force profiles) between the tissue and jaws. In certain circumstances, it may be advantageous to apply higher clamping forces on tissue sandwiched between the jaws 2502, 2504 where the lateral pressure gradients are small.

In exemplary form, each jaw 2502, 2504 may include one or more electrodes 2506 (one, two, three, four, five, etc.), with multiple electrodes being spaced apart from one another with respect to an outer perimeter across the convex profile. This second exemplary clamp style electrode configuration 2500 may be operated in a manner similar to or identical to that described for the foregoing clamp style electrode configuration 2400. Accordingly, for purposes of brevity, it is understood that the multiplex pulses and firing schemes discussed with respect to the electrodes of FIGS. 24B-24D is equally applicable to jaws 2502, 2504 incorporating multiple electrodes 2506.

Referencing specifically FIGS. 25B and 25C, though not required, one or both of the exemplary jaws 2502, 2504 may incorporate one or more ports 2512 in fluid communication with one or more embedded channels 2514, where the embedded channels are in fluid communication with at least one of a vacuum source (not shown) and an aspirating fluid source (not shown). By way of example, the ports 2512 may comprise one or more openings located in between adjacent electrodes 2506. When the embedded channels 2514 are in fluid communication with a vacuum source, suction may be applied via the ports as a means to pull the tissue against the electrodes 2406 and insulating substrate 2508. Moreover, applying vacuum to the tissue may be advantageous to draw bodily fluid away from the ablation site as a means to prohibit or reduce electrode arcing. In exemplary form, when both exemplary jaws 2502, 2504 include ports 2512 and embedded channels 2514 in communication with a vacuum source, the application of vacuum may be the primary means of drawing the jaws toward one another and clamping the tissue therebetween.

Likewise, the concave jaw 2504 may be utilized independent of the convex jaw 2502 to conduct PFA between counterpart electrodes 2506 with tissue interposing these electrodes to form one or more energy fields 2520.

In addition to operating the electrodes in accordance with the PFA protocols and schemes disclosed herein, it is also within the scope of the disclosure to utilize bidirectional ablation energy including radio frequency ablation before or after PFA. U.S. Pat. No. 9,924,998 is incorporated herein by reference. This patent describes utilization of opposing clamp jaws powered by a bidirectional energy source as a means to effectuate ablation of tissue. One can power the electrodes 2506 of the jaws as taught by U.S. Pat. No. 9,924,998, which is a form of ablation that results from tissue heating. Accordingly, this tissue heating may be managed by using the exemplary jaws 2502, 2504 so that cooling fluid from an aspirating fluid source may be directed through the embedded channels 2514 and egress through the ports 2512 in order to actively manage the temperature of the tissue between the jaws 2502, 2504. As those skilled in the art will realize, fluid egressing through the ports 2512 may be concurrently operative to cool not only the jaws themselves, but also the tissue interposing the jaws. In this manner, lateral heating resulting from bidirectional ablation extending to adjacent tissue that is not intended to be ablated may be mitigated or eliminated using active cooling.

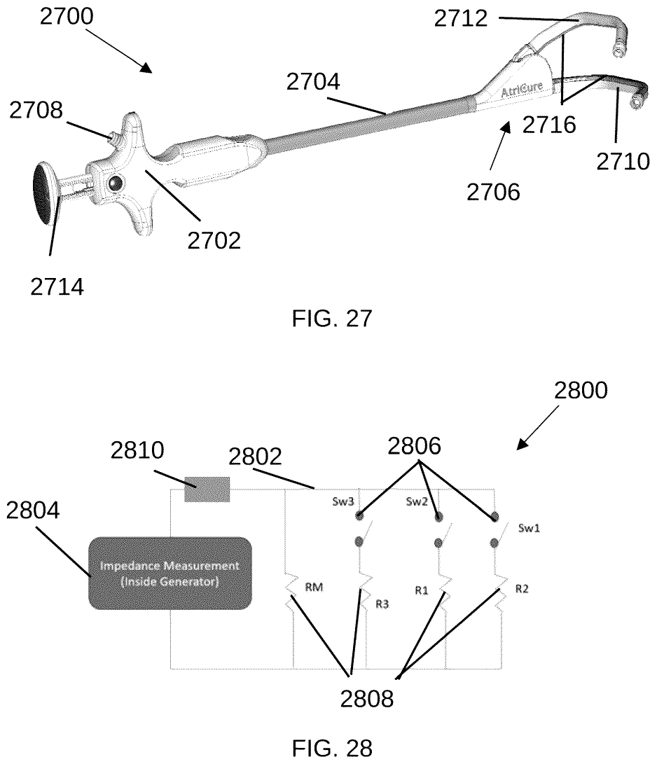

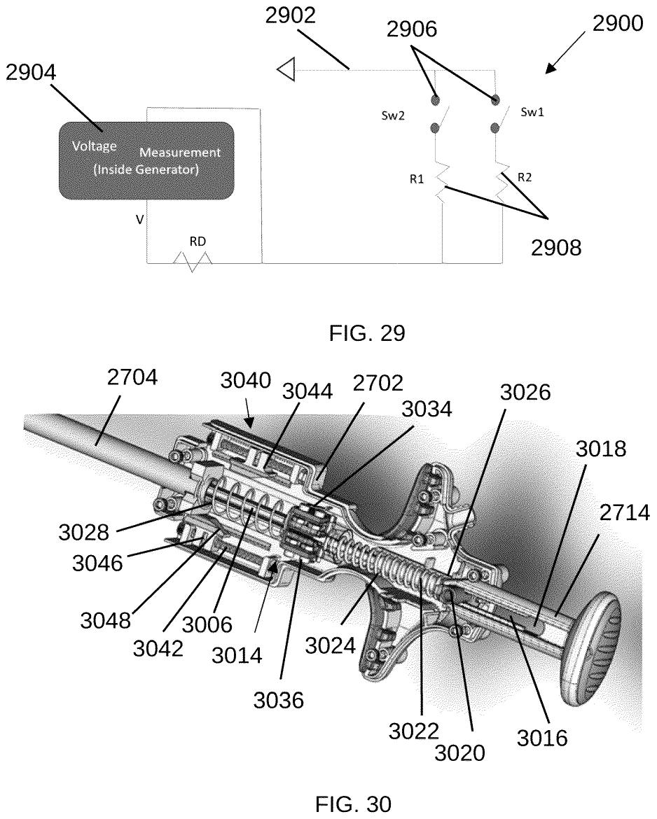

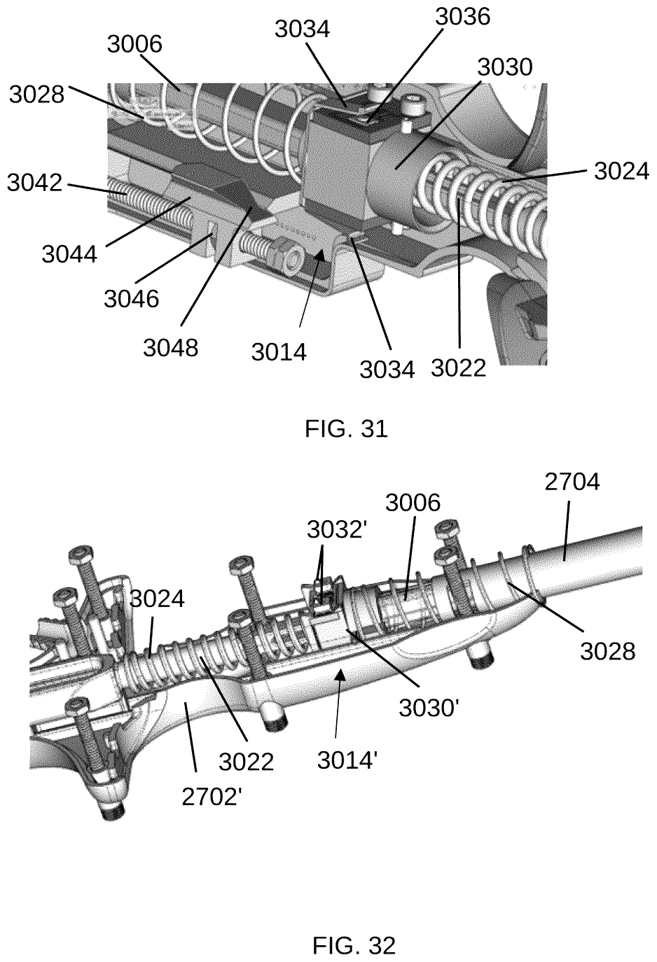

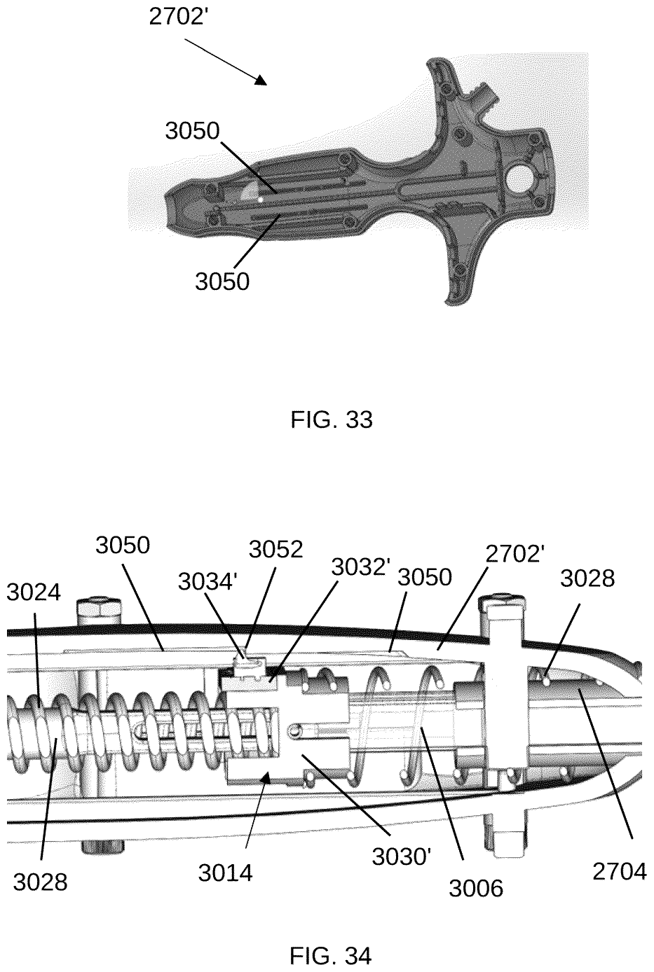

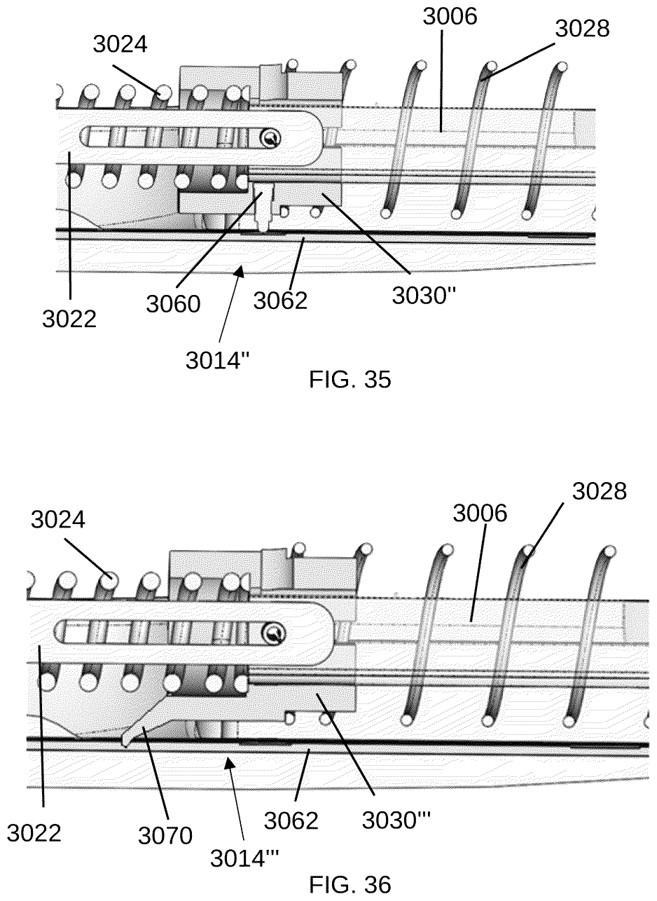

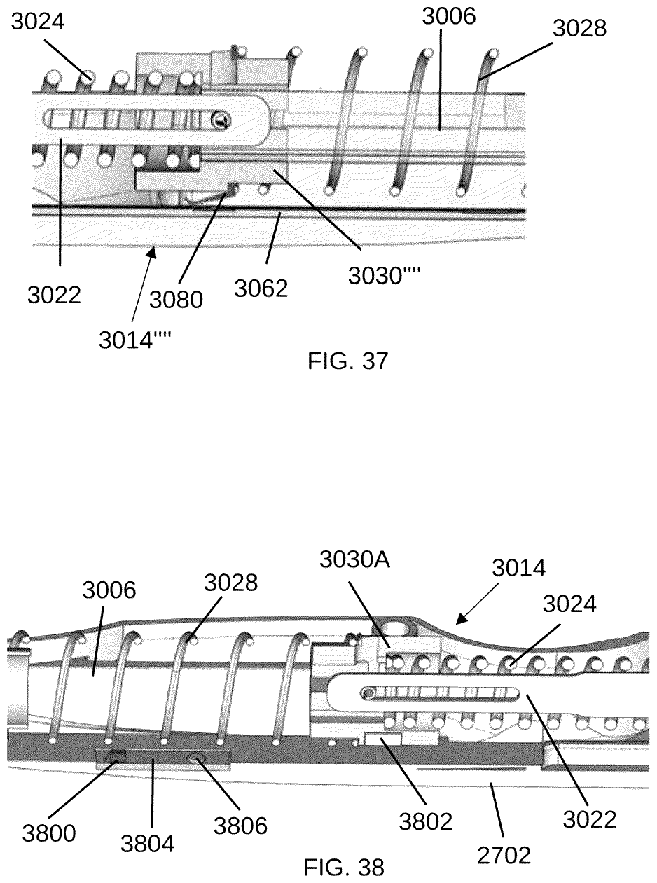

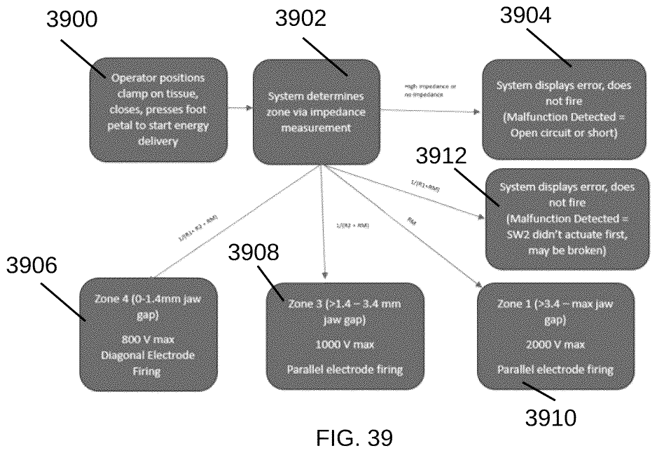

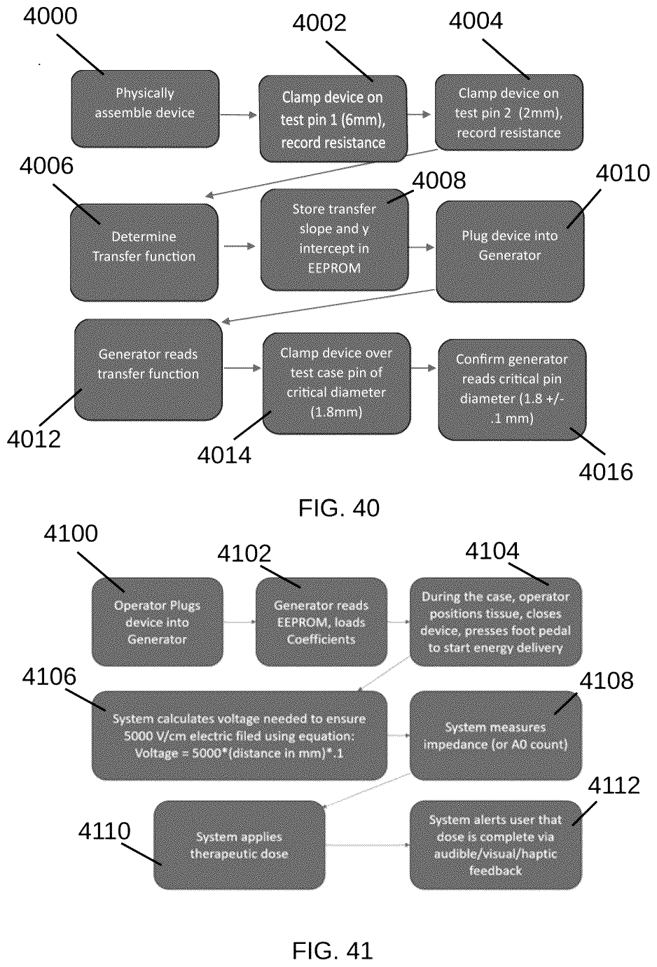

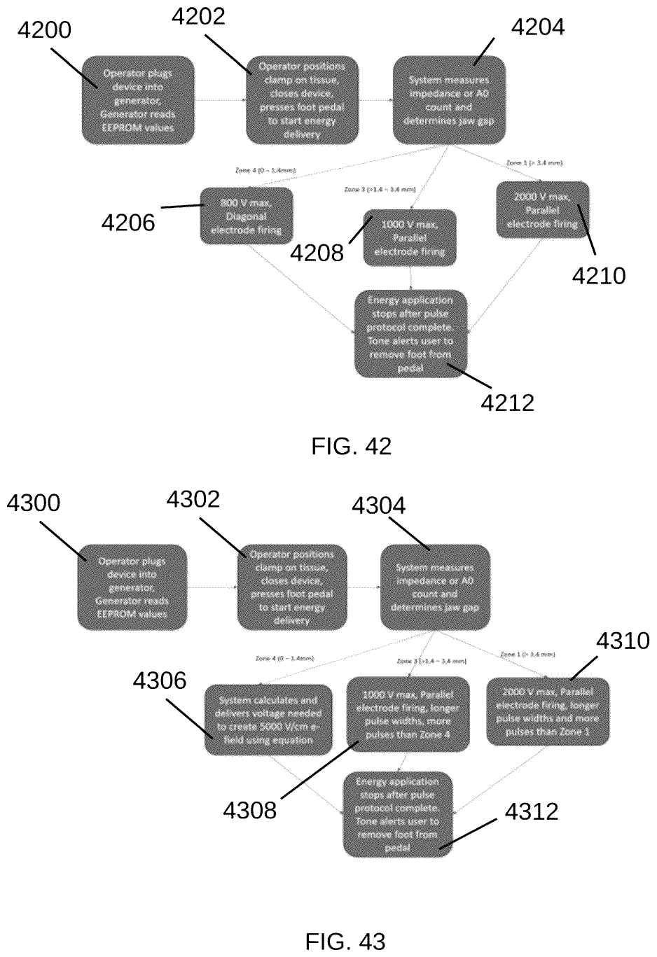

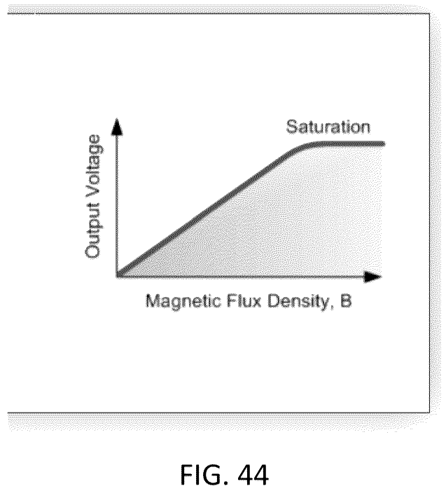

Referring to FIG. 26, in a still further example embodiment according to at least some aspects of the present disclosure, a clamp style electrode configuration 2600 may include a first jaw 2602 opposite a second jaw 2604, where the jaws are selectively opened and closed to vary the distance between electrodes 2606 on the jaws. In exemplary form, the electrodes 2606 may be embedded in an insulating substrate 2608 and include associated leads (not shown) in order to provide electrical communication with a PFA device (such as PFA device 104 as disclosed herein). By way of further example, the electrodes 2606 may extend along substantially the entire longitudinal length of the jaws 2602, 2604 or may extend for some distance shorter than the longitudinal length of the jaws. It should also be understood that the electrodes 2606 may comprise any number of segments distributed longitudinally. Moreover, the electrodes 2606 may have uniform widths, or may have widths that vary depending upon the position of the electrodes. For example, the widths of electrodes 2606 on the outer periphery may be wider than the widths of the electrodes toward the longitudinal center of the jaws 2602, 2604. Conversely, the widths of electrodes 2606 on the outer periphery may be narrower than the widths of the electrodes toward the longitudinal center of the jaws 2602, 2604. Moreover, the heights of electrodes 2606 extending above the insulating substrate 2608 on the outer periphery may be greater than the heights of the electrodes toward the longitudinal center of the jaws 2602, 2604. Further, the shape of electrodes 2606 extending above the insulating substrate 2608 may embody a rectangular cross-section along the longitudinal dimension, through various shapes of electrodes may be utilized. Still further, the shape of the electrodes 2606 (such as having rounded or bulbous longitudinal cross-section) on the outer periphery may be different from the shape of the electrodes (such as having a rectangular longitudinal cross-section) toward the longitudinal center of the jaws 2602, 2604. It is to be understood that the jaws 2602, 2604 may be used in lieu of the jaws 210, 212 as discussed herein and depicted in FIG. 2 for the exemplary PFA device 200 as part of clamping tissue between the jaws.

In an exemplary configuration, the insulating substrate 2608 of each jaw 2602, 2604 may exhibit a concave profile (longitudinal cross-section) that may be arcuate or have another indented shape so that at least one electrode 2606 extends outward from the insulating substrate. Having jaws 2602, 2604 with concave profiles that face one another may be advantageous in order to provide ports (not shown, but see FIG. 25C, for example) in communication with at least one of a vacuum source and an aspirating fluid source. This configuration may also be advantageous for providing a dedicated dimensioned space to accommodate tissue even when the jaws are fully pushed toward one another.

In exemplary form, each jaw 2602, 2604 may include one or more electrodes 2606 (one, two, three, four, five, etc.), with multiple electrodes being spaced apart from one another with respect to an outer perimeter across the concave profile. This exemplary clamp style electrode configuration 2600 may be operated in a manner similar to or identical to that described for the foregoing clamp style electrode configurations 2400, 2500. Accordingly, for purposes of brevity, it is understood that the multiplex pulses and firing schemes discussed with respect to the electrodes of FIGS. 24B-25C is equally applicable to jaws 2602, 264 incorporating multiple electrodes 2606.

In addition, the jaws 2602, 2604 may be operated with accordance with the operation of the jaws 2502, 2504 as described herein in the context of utilizing vacuum and/or cooling aspirating fluid.

Turning to FIGS. 27-38, a further exemplary clamp-type PFA device 2700 may include a proximally disposed handle 2702, a shaft 2704 extending distally from the handle 2702, and/or an end effector 2706 disposed distally on the shaft 2704. Generally, some example PFA devices 2700 may be similar to the “Isolator Synergy Encompass” surgical ablation device available from AtriCure, Inc. of Mason, Ohio, and/or the devices described in U.S. Pat. No. 11,678,928, issued Jun. 20, 2023, titled “SURGICAL CLAMP,” which is incorporated herein by reference in its entirety.

Generally, any handle described herein with reference to any exemplary embodiment may be configured to be grasped by a human user (e.g., surgeon) and/or engaged by a non-human, mechanical and/or robotic device (e.g., a surgical robot). More generally, any handle described herein may comprise any structure that may be configured to be secured, held, and/or manipulated to position and/or restrain a PFA device, regardless of whether it may be held by a human (e.g., surgeon or assistant), robot, mechanical device, etc.

In the illustrated embodiment, a proximally extending connecting element 2708 and associated line (not shown) may electrically couple the PFA device 2700 to the PFA unit 102 (see FIG. 1). In some embodiments utilizing vacuum and/or cryogenics, the connecting element 2708 may include suitable conduits. The end effector 2706, corresponding to end effector 132, may comprise a distal repositionable or fixed jaw 2710 and/or a movable proximal jaw 2712. A plunger 2714 or other actuator, which may be disposed proximally on the handle 2702, may allow the operator to reposition one or both jaw 2710, 2712 to clamp the target tissue 10 (see FIG. 1) between the jaws 2710, 2712. In the illustrated embodiment, one or both of the jaws 2710, 2712 may include one or more electrodes 2716, corresponding to the various electrodes disclosed herein, which may be utilized to deliver PFA energy to the target tissue 10. In embodiments including one or more electrodes, the electrode(s) may be positioned on either or both jaws 2710, 2712. In the context of using electrodes on opposing jaws and performing PFA, it is important to know the distance between the electrodes of the jaws and the jaws themselves. What follows are a number of exemplary solutions to determining the spacing between the jaws and jaw electrodes of a PFA device.

FIG. 28 depicts an exemplary circuit diagram 2800 utilizing a plurality of continuity switches to determine the distance between the jaws of a PFA clamp device (including any of the foregoing disclosed PFA clamp devices herein) after the jaws have clamped tissue therebetween. Once the distance between the clamp device jaws is determined, the voltage, number of pulses, dwell times, pulse durations, sequence of electrode activation, etc. (collectively referred to as the dose) may be implemented by the PFA unit 102 as described herein. In a preferred dose, tissue efficacy is maximized while avoiding potential failure modes such as arcing or undesirable thermal impact to the tissue. As used herein, a “zone” of operation corresponds a particular distance range between jaws of a PFA device. In exemplary form, software running on the PFA unit 102 may interpret which zone the jaws of the PFA device are in and thereafter determine what dose will be implemented. By way of example, in a case of seven zones, the zones denote progressively different spacing between the jaws with zone 1 being the largest distance between the jaws, while zone 7 is the smallest distance between the jaws, with the zones therebetween being consecutively numbered as the spacing incrementally changes. It should be noted that the circuit diagram may incorporate fewer switches than three or more switches than three, and likewise incorporate fewer resistors and corresponding legs. Based upon the following description, those skilled in the art will understand how the exemplary circuit diagram 2800 can be scaled up or scaled down depending upon the number of zones desired.

The exemplary circuit diagram 2800 represents one or more printed circuit boards (PCBs) 2802 in electrical communication with an impedance monitoring circuit 2804 associated with the PFA unit 102, where the PCBs 2802 include electrical continuity switches 2806 in series with resistors 2808 of known resistance, where the switches and resistors are placed in parallel with one another. While three resistors 2808 in series with corresponding switches 2806 are shown in FIG. 28, it should be noted that more than three or fewer than three switch-resistor series combinations may be utilized. As the number of switch-resistor series combinations is increased, so too is the number of zones increased. And as the number of zones is increased, so too is the precision of the jaw distance measurements because each zone corresponds to a small range of motion (i.e., smaller distance range between the jaws). It should also be noted that as the number of switch-resistor series combinations increase, so too should the resistance of each resistor be distinct from one another so that no two resistances are indistinguishable for purposes of impedance measurement.

In exemplary form, as depicted in Table 1 below, if the PFA unit 102 receives no signal from the PCBs 2802 (or analog to digital converter 2810), the PFA clamp device is either off (not powered) or, if on, a fault has occurred (likely an open circuit). In such a circumstance, the PFA unit 102 will be unable to determine the spacing between the jaws. Similarly, if the PFA unit 102 receives a signal and determines the impedance is beyond a predetermined measurement threshold, then a fault condition is present and the PFA unit 102 will be unable to determine the spacing between the jaws of the PFA clamp device. In a circumstance where the PFA unit 102 does receive a signal from the PCBs 2802 (or analog to digital converter 2810), the PFA unit will utilize this signal to determine the spacing between the jaws of the PFA clamp device.

TABLE 1

Malfunction Detection: No impedance in system (short)
Malfunction Detection: High Impedance (open circuit)
Zone 1: Impedance = RM (No Switches closed)
Zone 2: Impedance = 1/(R1 + RM)
Zone 3: Impedance = 1/(R2 + RM)
Zone 4: Impedance = 1/(R3 + RM)
Zone 5: Impedance = 1/(R1 + R2 + RM)
Zone 6: Impedance = 1/(R1 + R3 + RM)
Zone 7: Impedance = 1/(R2 + R3 + RM)

By way of example, as depicted in Table 1 above, if the PFA unit 102 receives a signal from the PCBs 2802 (or analog to digital converter 2810) indicative of the PFA clamp device being powered, but none of the switches 2806 being closed, the signal will be changed by the impedance of the closed circuit. In such a case, a zone 1 condition is present, indicative of the jaws being fully open or nearly fully open and tissue is clamped therebetween, with the impedance of the circuit being determined by the resistance of resistor RM. In case where a zone 2 condition is present, the jaws are closer together than zone 1, but farther apart in comparison to zones 3-7. When a zone 2 condition is present, the PFA unit 102 receives a signal from the PCBs 2802 (or analog to digital converter 2810) indicative of the PFA clamp device being powered and switch SW2 being closed. In this fashion, the current travels in parallel through the RM leg of the circuit as well as through the resistor R1 and switch SW2 leg of the circuit. And the impedance is calculated just as one would calculate resistance through a parallel circuit as being the inverse of the resistance of each leg of the circuit. In a circumstance where a zone 3 condition is present, the jaws are closer together than zone 2, but farther apart in comparison to zones 4-7. When a zone 3 condition is present, the PFA unit 102 receives a signal from the PCBs 2802 (or analog to digital converter 2810) indicative of the PFA clamp device being powered and switch SW1 being closed. In this fashion, the current travels in parallel through the RM leg of the circuit as well as through the resistor R2 and switch SW1 leg of the circuit. And the impedance is calculated through these parallel legs of the circuit. In a state where a zone 4 condition is present, the jaws are closer together than zone 3, but farther apart in comparison to zones 5-7. When a zone 4 condition is present, the PFA unit 102 receives a signal from the PCBs 2802 (or analog to digital converter 2810) indicative of the PFA clamp device being powered and switch SW3 being closed. In this fashion, the current travels in parallel through the RM leg of the circuit as well as through the resistor R3 and switch SW3 leg of the circuit. And the impedance is calculated through these parallel legs of the circuit. But the calculation is slightly different for zones 5-7.

By way of further example, as depicted in Table 1 above, if the PFA unit 102 receives a signal from the PCBs 2802 (or analog to digital converter 2810) indicative of the PFA clamp device being powered, and zone 5 condition is present, indicative of the jaws being closer together than zone 4, but farther apart in comparison to zones 6 and 7, the impedance of the circuit was determined using the resistance of resistors RM, R1, R2 because the switches SW1 and SW2 were closed. In case zone 5 condition, the impedance is calculated just as one would calculate resistance through a parallel circuit as being the inverse of the resistance of each of the three legs of the circuit. Similarly, if the PFA unit 102 receives a signal from the PCBs 2802 (or analog to digital converter 2810) indicative of the PFA clamp device being powered, and zone 6 condition is present, indicative of the jaws being closer together than zone 5, but farther apart in comparison to zone 7, the impedance of the circuit was determined using the resistance of resistors RM, R1, R3 because the switches SW2 and SW3 were closed. Finally, if the PFA unit 102 receives a signal from the PCBs 2802 (or analog to digital converter 2810) indicative of the PFA clamp device being powered, and zone 7 condition is present, indicative of the jaws being as closely spaced from one another with tissue therebetween, but farther apart in comparison to zone 6, the impedance of the circuit was determined using the resistance of resistors RM, R2, R3 because the switches SW1 and SW3 were closed.

Referencing FIG. 29, it is also within the scope of the disclosure to measure impedance across one or more PCBs 2902 using a voltage measurement utilizing a plurality of continuity switches to determine the distance between the jaws of a PFA clamp device (including any of the foregoing disclosed PFA clamp devices herein) after the jaws have clamped tissue therebetween. Once the distance between the clamp device jaws is determined, the voltage, number of pulses, dwell times, pulse durations, sequence of electrode activation, etc. (collectively referred to as the dose) may be implemented by the PFA unit 102 as described herein.

The exemplary circuit diagram 2900 represents one or more printed circuit boards (PCBs) 2902 in electrical communication (and in series) with an impedance monitoring circuit 2904 with its own resistor RD associated with the PFA unit 102, where the PCBs 2902 include electrical continuity switches 2906 in series with resistors 2908 of known resistance, where the switches and resistors are placed in parallel with one another. While two resistors 2908 in series with corresponding switches 2906 are shown in FIG. 29, it should be noted that more than three switch-resistor series combinations may be utilized. As the number of switch-resistor series combinations is increased, so too is the number of zones increased. And as the number of zones is increased, so too is the precision of the jaw distance measurements because each zone corresponds to a small range of motion (i.e., smaller distance range between the jaws). It should also be noted that as the number of switch-resistor series combinations increase, so too should the resistance of each resistor be distinct from one another so that no two resistances are indistinguishable for purposes of impedance measurement.

In exemplary form, as depicted in Table 2 below, if the PFA unit 102 receives no voltage from the PCBs 2802, the PFA clamp device is either off (not powered) or, if on, a fault has occurred (likely an open circuit). In such a circumstance, the PFA unit 102 will be unable to determine the spacing between the jaws. Conversely, if the PFA unit 102 receives a voltage, the PFA unit will utilize this voltage to determine the spacing between the jaws of the PFA clamp device.

TABLE 2

Malfunction Detection: No Voltage (open circuit)
Zone 1: Voltage = V [symbol] (No switch active)
Zone 2: Voltage = V * R1/(R1 + RD)
Zone 3: Voltage = V * R2/(R2 + RD)
Zone 4: Voltage = V * [1/(R1 + R2)]/([1/(R1 + R2)] + RD)

By way of example, as depicted in Table 2 above, if the PFA unit 102 receives a voltage from its own circuit indicative of the PFA clamp device being powered, but none of the switches 2906 being closed, the voltage V will be unchanged as the current is only traveling through resistor RD. In such a case, a zone 1 condition is present, indicative of the jaws being fully open or nearly fully open and tissue is clamped therebetween. In case where a zone 2 condition is present, the jaws are closer together than zone 1, but farther apart in comparison to zones 3-73 and 4. When a zone 2 condition is present, the PFA unit 102 receives voltage from its internal circuit and from the PCBs 2902 indicative of the PFA clamp device being powered and switch SW2 being closed. In this fashion, the current travels in series through the internal circuit as well as through the resistor R1 and switch SW2 portion of the circuit. And the voltage is calculated just as one would calculate voltage through a series circuit as being the voltage V multiplied by the resistance of resistor R1, divided by the total resistance (R1+RD). In a circumstance where a zone 3 condition is present, the jaws are closer together than zone 2, but farther apart in comparison to zone 4. When a zone 3 condition is present, the PFA unit 102 receives voltage from its internal circuit and from the PCBs 2902 indicative of the PFA clamp device being powered and switch SW1 being closed. And the current travels in series through the internal circuit as well as through the resistor R2 and switch SW1 portion of the circuit. Voltage is calculated just as one would calculate voltage through a series circuit as being the voltage V multiplied by the resistance of resistor R2, divided by the total resistance (R2+RD). Finally, when a zone 4 condition is present, the PFA unit 102 receives voltage from its internal circuit and from the PCBs 2902 indicative of the PFA clamp device being powered and switches SW1 and SW2 being closed. And the current travels in series through the internal circuit as well as in parallel through the resistor R2 and switch SW1 and resistor R1 and switch SW2 portion of the circuit. Voltage is calculated just as one would calculate voltage through a series and parallel circuit as being the voltage V multiplied by the inverse of resistance of resistors R1 and R2, divided by the sum of the inverse of resistors R1 and R2 and the resistance of resistor RD.

Turning to FIGS. 30 and 31, implementation of the foregoing alternatives to determine jaw spacing can be carried out in a multitude of acceptable fashions. FIG. 30 provides a view of the internal components of the handle 2702 in a first exemplary manner. An interior of the handle 2702 may house an actuator mechanism 3014. In this example embodiment, the plunger 2714 may be used to articulate one or more of the jaws of a PFA device, such as those disclosed herein. The plunger 2714 may be generally aligned with the shaft 2704. With the plunger 2714 in the fully retracted or proximal position, the jaws may be in a fully open position (see, e.g., FIG. 27). When the plunger 2714 is depressed in the distal direction, at least one of the jaws of the PFA device may move toward the other jaw and toward a closed or clamping position.

In some example embodiments, the actuator mechanism 3014 may include a locking mechanism. For example, the plunger 2714 may include a generally longitudinal slot 3016 with a widened proximal opening 3018. When the jaws of the PFA device are in the closed position, the opening 3018 may align with a lock button 3020, which may be spring-biased to drive the lock button into the opening 3018, thereby preventing the plunger 2714 from moving proximally and maintaining the jaws in the closed position. Depressing the lock button 3020 may disengage the lock button 3020 from the opening 3018, thereby releasing the plunger 2714 and allowing it to move proximally to open the jaws.

In some example embodiments, the actuator mechanism 3014 may be configured to control and/or limit the amount of force that may be applied by the jaws of the PFA device when the plunger 2714 is depressed. For example, the actuator mechanism 3014 may include a relief rod 3022 and a force limiting spring 3024. The relief rod 3022 may be slidable with respect to the actuator linkage 3006, while the force limiting spring 3024 may be arranged to apply a distal force to the actuator linkage 3006. As the plunger 2714 is depressed, the force limiting spring 3024 may compressed between a step 3026 on the plunger 2714 and the actuator linkage 3006. Accordingly, depressing the plunger 2714 imparts a load on the force limiting spring 3024 that is transferred to the actuator linkage 3006, which moves the actuator linkage 3006 distally. If the jaw clamping load exceeds the desired maximum while the plunger 2714 continues to be depressed, the force limiting spring 3024 is further compressed and the relief rod 3022 moves distally without moving the actuator linkage 3006. Thus, the force limiting spring 3024 substantially limits the maximum jaw clamping load. One with ordinary skill in the art will recognize that the tissue clamping pressure may be a function of the jaw clamping force and the tissue area being clamped. The actuator mechanism 3014 may include a return spring 3028 that may be operative to move the actuator linkage 3006 proximally upon releasing the plunger 2714.

In exemplary form, the actuator mechanism 3014 may include a housing 3030 mounted to and receiving a distal end of the force limiting spring 3024 and concurrently mounted to a proximal end of the actuator linkage 3006. On the top and bottom of the housing 3030 are mounted opposing mechanical switches 3032, where each mechanical switch includes a flexible arm 3034 biased away from an electrical contact 3036. When the flexible arm 3034 comes into contact with the electrical contact 3036, the switch is closed and allows electrical contact through the switch. While not shown, each switch 3032 includes leads to establish electrical communication with a PFA unit 102 when the switch is closed.

Inside the handle 2702 are two top and bottom cavities interposed by the actuator linkage 3006 and the return spring 3028. These cavities house respective interference features 3040, where each interference feature includes a threaded bolt 3042, a carriage 3044, and a nut 3046. By way of example the handle 2702 includes retainers that the threaded bolt 3042 is seated within and that allow the threaded bolt to rotate in place. The carriage 3044 includes a pair of legs that are interposed by the nut 3046. In this fashion, the pair legs delineate opening that receive the threaded bolt. In exemplary form, these openings may be threaded to obviate the nut, or may be non-threaded. In any event, the openings of the legs receive the nut 3046 therebetween and the legs and nut receive the threaded bolt 3042 so that rotation of the threaded bolt is operative to reposition the nut and correspondingly the carriage 3044 longitudinally along the threaded bolt. In exemplary form, each carriage includes a sloped surface 3048 that is configured to face a respective flexible arm 3034 of a respective switch 3032. Depending upon when (at what depth of distal repositioning of the plunger 2714) the designer intends to have the respective switches 3032 closed, upon assembly of the PFA device, the designer may rotate the threaded bolts to the properly longitudinally position each carriage 3044. In this manner, as the plunger 2714 is repositioned distally, and causes the housing 3030 to correspondingly be repositioned distally, and with it the switches 3032, at least one of the flexible arms 3034 is intended to be contacted by a sloped surface 3048 of a carriage 3044 and eventually compress the flexible arm to make contact with its corresponding electrical contact 3036 and close the switch in question. After passing beyond the sloped surface distally, the carriage includes a top plateau that retains the switch in a closed position for a predetermined range of distal travel of the plunger 2714. It is anticipated that the carriages 3044 are staggered longitudinally so that successive switches are closed and/or opened in sequence.

As depicted in FIGS. 32-34, an alternate exemplary actuator mechanism 3014′ may include a housing 3030′ mounted to and receiving a distal end of the force limiting spring 3024 and concurrently mounted to a proximal end of the actuator linkage 3006. On the top of the housing 3030′ are mounted two mechanical switches 3032′, where each mechanical switch includes a repositionable fin 3034′ biased away from an internal electrical contact (not shown). Mechanical switches 3034′ of this sort are commercially available as the C&K HDT0001 Switch, part number HDT0001, stock number 70417475, from RS Americas, Inc. (https://us.rs-online.com/). When the repositionable fin 3034′ comes into contact with the internal electrical contact, the switch is closed and allows electrical contact through the switch. While not shown, each switch 3032′ includes leads to establish electrical communication with a PFA unit 102 when the switch is closed.

Inside the handle 2702′ are two grooves 3050, each configured to align with one of the responsible fins 3034′. These grooves 3050 include step changes in height so that when a repositionable fin 3034′ is seated therein, the fin may be upstanding as shown in FIG. 34. But when the housing 3030′ is longitudinally repositioned beyond a predetermined point, a step change 3052 in the groove forces a respective fin 3034 to become repositioned and makes contact with its internal electrical contact, thereby closing the switch 3032′. Depending upon when (at what depth of distal repositioning of the plunger 2714, see FIG. 27)) the designer intends to have the respective switches 3032 closed, designer configures the handle 2702′ grooves 3050 and the step change 3052 to correspond to a predetermined position of the plunger. In this manner, as the plunger 2714 is repositioned distally, and causes the housing 3030′ to correspondingly be repositioned distally, and with it the switches 3032′, at least one of the repositionable fins 3034′ is intended to come into eventual contact with the step change 3052, thereby repositioning the fin 3034′ to make contact with its corresponding electrical contact and close the switch in question. After passing beyond the step change 3052 distally, the repositionable fin 3034′ may continue to be depressed or may transition to another area where further distal motion allows the fin to extend and no longer make contact with the internal electrical contact, thereby opening the switch. This same procedure is carried out for each switch 3032′ and corresponding groove 3050, where the depth and position of the step change(s) 3052 varies longitudinally depending upon when the designer intends the switch to be closed based upon the longitudinal position of the plunger.

Referencing FIGS. 35-37, it is also within the scope of the disclosure to determine the spacing between jaws of a PFA device using at least one sealed linear membrane potentiometer. Sealed membrane potentiometers are superior to other potentiometer designs because they are completely enclosed systems. Sealed membrane potentiometers are also not subject to shorting via fluid ingress or sensor drift via dust/humidity.

In exemplary form, three further alternate exemplary actuator mechanism 3014″, 3014′″, 3014″″ may include respective housings 3030″, 3030′″, 3030′″ that are mounted to and receive a distal end of the force limiting spring 3024 and concurrently mounted to a proximal end of the actuator linkage 3006. A first alternate exemplary housing 3030″ includes a pogo pin 3060 seated therein and extending downward to engage a linear potentiometer 3062. A second alternate exemplary housing 3030′″ includes a cantilever finger 3070 integrally formed with the housing and extending downward to engage a linear potentiometer 3062. A third alternate exemplary housing 3030′″ includes a spring arm 3080 mounted t the underside of the housing and extending downward to engage a linear potentiometer 3062. In each of the foregoing alternate exemplary embodiments, the linear potentiometer 3062 includes electrical leads in communication with the PFA unit 102. As the pogo pin 3060, cantilever finger 3070, or spring arm 3080 engages the linear potentiometer 3062, engagement with the linear potentiometer in different longitudinal locations results in different voltages generated by the linear potentiometer, which can be correlated to the position of the plunger 2714 and, correspondingly, to the spacing of the jaws of the PFA device from one another.

Referring to FIG. 38, it is also within the scope of the disclosure to determine the spacing between jaws of a PFA device 2700 using at least one Halls Effect sensor 3800. Those skilled in the art are familiar with Halls Effect sensors. By way of explanation and reference to FIG. 44, Halls Effect sensors 3800 can be described using the following equation,

$${V}_{H} = {R}_{H} ( \frac{I}{t} \times B )$$

where V_{H} is the Hall voltage in volts, Ru is the Hall Effect coefficient, I is the current in amperes, t is the thickness of the sensor in millimeters, and B is the magnetic flux in Teslas. In exemplary form, the Halls Effect sensor 3800 may include at least three conductors communicating with the PFA unit 102 (see FIG. 1), where these three conductors include a ground conductor, an input voltage conductor, and an output voltage conductor. By way of example, the PFA unit 102 supplies a fixed input voltage to the Halls Effect sensor 3800 via the input voltage conductor and monitors the output voltage from the Halls Effect sensor via the output voltage conductor.

In order to use the Halls Effect sensor 3800 to determine spacing between the jaws 2710, 2712, for example) of a PFA device (e.g., 2700, see FIG. 27), the interior of the handle 2702 houses an actuator mechanism 3014 that may be configured to control and/or limit the amount of force that may be applied by the jaws of the PFA device when the plunger 2714 (see FIG. 27) is depressed. While this alternate exemplary embodiment uses a Halls Effect sensor 2800 and other components in place of those disclosed herein to determine the distance between jaws of a PFA device, common reference numerals refer to common elements and should be understood as such.

For example, the actuator mechanism 3014 may include a relief rod 3022 and a force limiting spring 3024. The relief rod 3022 may be slidable with respect to the actuator linkage 3006, while the force limiting spring 3024 may be arranged to apply a distal force to the actuator linkage 3006. As the plunger 2714 is depressed, the force limiting spring 3024 may compressed between a step on the plunger 2714 and a housing 3030A. Accordingly, depressing the plunger 2714 imparts a load on the force limiting spring 3024 that is transferred to the actuator linkage 3006 via the housing 3030A, which moves the actuator linkage 3006 distally. If the jaw clamping load exceeds the desired maximum while the plunger 2714 continues to be depressed, the force limiting spring 3024 is further compressed and the relief rod 3022 moves distally without moving the actuator linkage 3006. Thus, the force limiting spring 3024 substantially limits the maximum jaw clamping load. One with ordinary skill in the art will recognize that the tissue clamping pressure may be a function of the jaw clamping force and the tissue area being clamped. The actuator mechanism 3014 may include a return spring 3028 that may be operative to move the actuator linkage 3006 proximally upon releasing the plunger 2714.

The actuator mechanism 3014 includes the housing 3030 mounted to and receiving a distal end of the force limiting spring 3024 and concurrently mounted to a proximal end of the actuator linkage 3006. On the bottom of the housing 3030A is mounted a permanent magnet 3802 that is fixed in position with respect to the housing, but traverses proximally and distally as the housing traverses. A printed circuit board (PCB) 3804 may be fixedly mounted to an interior of the handle 2702, where the PCB includes an opening configured to receive an alignment post 3806 extending from the interior of the handle 2702. This alignment post ensures the PCB 3804 remains stationary and sufficiently distal to the range of motion of the housing 3030A. More specifically, in exemplary form, the Halls Effect sensor 2800 is mounted to the PCB 3804 in a position sufficiently distal to ensure that the magnet 3802 approaches the Halls Effect sensor 3800 from a single direction (i.e., the magnet does not approach, pass over, and continue beyond the Halls Effect sensor in a singular direction). Simply put, the location of the Halls Effect sensor 3800 may be more distal than the distal-most range of travel of the magnet 3802.

In exemplary form, as the distance between the magnet 3802 and the Halls Effect sensor 3800 changes, so too will the output voltage from the Halls Effect sensor change. In this manner, the PFA unit 102 monitors the output voltage from the Halls Effect sensor via the output voltage conductor and calculates the change in distance between the magnet and the Halls Effect sensor, which is directly related to the change in distance between the jaws 2710, 2712 of the PFA device 2700. In particular, the voltage changes output from the Halls Effect sensor 3800 are proportional to the distance changes occurring between the jaws of the PFA device.

It is also within the scope of the disclosure to switch the positions of the Halls effect sensor 3800 and the magnet 3802. Specifically, the magnet 3802 may be fixedly mounted to the interior of the handle 2702 in a position more distal than the distalmost range of travel of the housing 3030A to which the Halls Effect sensor 3800 is fixedly mounted.

Referring to FIGS. 27 and 40, in accordance with the instant disclosure, it may be advantageous to conduct a calibration sequence to calibrate the continuity switches 2806, 2906, the mechanical switches 3032 (including the prime alternatives), the potentiometer 3062, and/or the Halls Effect sensor 3800. With the PFA device 700 fully assembled (completion of step 4000), the device jaws 2710, 2712 can be clamped on two or more gauge pins each having a known diameter. For the sake of this example, one can use a first pin having a 6 millimeter diameter and second pin having a 2 millimeter diameter. In this manner, the PFA device 2700 is operated to reposition the jaws 2710, 2712 to clamp the first test pin in step 4002 and record the resistance of the potentiometer 3062, or record the output voltage of the Hall Effect sensor 3800, or record the impedance or the voltage for the continuity switches 2806, 2906, 3032. Thereafter, the PFA device 2700 is operated to reposition the jaws 2710, 2712 to clamp the second test pin in step 4004 and record the resistance of the potentiometer 3062, or record the output voltage of the Hall Effect sensor 3800, or record the impedance or the voltage for the continuity switches 2806, 2906, 3032.

Regardless of the methods and structures utilized to determine the distance or spacing between the jaws 27110, 2712 of the PFA device 2700, one may utilize or generate a transfer function in step 4006 that correlates the distance between the jaws and the output from the continuity switches 2806, 2906, the mechanical switches 3032 (including the prime alternatives), the potentiometer 3062, and/or the Halls Effect sensor 3800. Specifically, because the change in output has a linear relationship (or other known relationship) to a change in distance between the jaws, a mathematical representation that describes this relationship may be established from outputs from steps 4002, 4004. After determining the transfer function in step 4006, the slope and Y-intercept may be stored in the electrically erasable programmable read-only memory (EEPROM) of the PFA device 2700 in step 4008. Similarly, in circumstances where the relationship is or is not linear, the transfer function may be stored on the EEPROM in step 4008.

After programming or storing the transfer function into the EEPROM of the PFA device 2700 in step 4008, the PFA device may be connected to the PFA unit 102 in step 4010. Thereafter, the PFA unit 102 may access the EEPROM of the PFA device 2700 in step 4012 to obtain the transfer function (or other mathematical relationship information stored providing a correlation between jaw 2710, 2712 distance and outputs from the PFA device meter/switch/sensor). This allows the PFA unit 102 to predict the jaw gap very accurately for that specific device despite the positional tolerances of the jaws and meter/switch/sensor associated with the PFA device 2700. This predicted jaw gap may be tested in step 4014 by having the PFA device 2700 clamp a third test pin having a 1.8 millimeter diameter. In step 4016, the PFA unit 102 uses the transfer function or stored mathematical information to and the output from the PFA device 2700 sensor/meter/switch (2806, 2906, 3032, 3062, 3800) to calculate the clamped jaw distance/gap and compare this calculated distance against the known test distance of 1.8 millimeters. Presuming the calculated distance is within plus or minus 0.1 millimeters of the 1.8 millimeter known distance, the PFA device 2700 is considered calibrated and ready for final packaging. In the alternative, however, the calculated distance is outside of plus or minus 0.1 millimeters of the 1.8 millimeter known distance, a new transfer function is generated based upon the outputs generated from the PFA device 2700 in steps 4002, 4004, 4014. Thereafter, the new transfer function is utilized by the PFA unit 102 to predict the jaw gap output(s) for a jaw distance of 1.8 millimeters. The PFA device 2700 than clamps its jaws around the 1.8 millimeter pin and determines the output(s) from the PFA device. If these outputs reflect a jaw distance of 1.8 millimeters plus or minus 0.1 millimeters, the PFA device is considered calibrated and ready for final packaging. If not, the PDF device may undergo additional testing to determine whether components of the PFA device 2700 are functioning correctly and, if so, additional revisions to the transfer function may be made until the outputs of the PFA device reflect the test gap of 1.8 millimeters within acceptable tolerances.

Turning to FIG. 39, as discussed herein, after the distance between the PFA device 2700 jaws 2710, 2712 is determined (with the tissue clamped therebetween and occupying this distance), the PFA parameters/dose may be determined that include, without limitation, the voltage, number of pulses, dwell times, pulse durations, sequence of electrode activation, etc. Even the energy modality may change based on the PFA device 2700 jaw gap. For example, tissue that results in jaw gaps greater than 6.5 mm, compressed, may be treated with RF energy instead of PFA due to the probability of fat. The goal is to select a dose that maximizes tissue efficacy while avoiding potential failure modes such as arcing or undesirable thermal impact to the tissue.

Turning to FIG. 39, a first exemplary scheme to maximize efficacy, while minimizing the risk of arcing, may include dynamically altering the maximum voltage based on the determined jaw gap from tissue clamped therebetween. In step 3900, the operator establishes communication between the PFA unit 102 and the PFA device 2700, clamps tissue between the PFA device, and begins the flow of energy to the electrodes. The PFA unit 102 may then calculate impedance measurements between counterpart electrodes in step 3902. If, for whatever reason, the impedance measurement is not completed for a given set of counterpart electrodes, the system determines a malfunction has occurred with respect to those counterpart electrodes in step 3904. But for counterpart electrodes where impedance can be measured, and it is determined that the gap between the jaws of the device is no more than 1.4 millimeters, step 3906 includes the PFA unit 102 powering a first set of electrodes corresponding to a distinct zone 4 using diagonal electrode firing, 800 volts maximum. Again, for counterpart electrodes where impedance can be measured, and it is determined that the gap between the jaws of the device is greater than 1.4 millimeters and no more than 3.4 millimeters, step 3908 includes the PFA unit 102 powering a second set of electrodes corresponding to a distinct zone 3 using parallel electrode firing, 1000 volts maximum. And for counterpart electrodes where impedance can be measured, and it is determined that the gap between the jaws of the device is greater than 3.4 millimeters, step 3910 includes the PFA unit 102 powering a third set of electrodes corresponding to a distinct zone 1 using parallel electrode firing, 2000 volts maximum. In a case where a corresponding fourth set of electrodes, corresponding to zone 2, is able to be used for an impedance measurement, but the electrodes do not fire or some other issue occurs that precludes delivery of energy to the electrodes, then the PFA unit 102 in step 3912 returns an error signal and notifies the operator that the ablation of zone 2 was ineffective. This signal may be audible, visual, and/or haptic.

Referring to FIG. 41, an alternative scheme to maximize efficacy, while minimizing the risk of arcing, may include dynamically altering the maximum voltage based on the determined jaw gap from tissue clamped therebetween. In step 4100, the operator establishes communication between the PFA unit 102 and the PFA device 2700. The PFA unit 102 may then read the memory of the PFA device 2700 to obtain the transfer function or other mathematical relationship information to correlate outputs from the PFA device sensor/meter/switch (2806, 2906, 3032, 3062, 3800) into distance determinations (i.e., jaw spacing) in step 4102. During medical use in step 4104, the PFA device 2700 is utilized to clamp tissue between the jaws 2710, 2712 and energy delivery to the electrodes of the jaws commences. In step 4106, the PFA unit 102 may calculate the voltage necessary to accomplish a 5000 Volts/centimeter electric field, based on the assumption that 5000 V/cm is the nominal electric field target and 2000 V is the maximum that the PFA device 102 can deliver. Thereafter, in step 4108, the PFA unit 102 may calculate impedance (or AO count if an analog to digital converter is being utilized), changes in voltage, etc., and from this calculation, in step 4110 the PFA unit may deliver energy to the PFA device in a dose sufficient to ablate the tissue clamped therebetween. When the dose is delivered sufficiently to ablate the tissue clamped therebetween, the PFA unit 102 may alert an operator of the PFA device 2700 in the form of an audible, visual, and/or haptic feedback in step 4112.

Referencing FIG. 42, a further alternate scheme is represented to maximize efficacy, while minimizing the risk of arcing is to use the resolution of the measurement, but operate in three or more tissue ablation zones. In step 4200, the operator establishes communication between the PFA unit 102 and the PFA device 2700 so as to allow the PFA unit 102 to read the memory of the PFA device 2700 to obtain the transfer function or other mathematical relationship information to correlate outputs from the PFA device sensor/meter/switch (2806, 2906, 3032, 3062, 3800) into distance determinations (i.e., jaw spacing). During medical use in step 4202, the PFA device 2700 is utilized to clamp tissue between the jaws 2710, 2712 and energy delivery to the electrodes of the jaws commences based upon zones of the electrodes and tissue to be ablated. In step 4204, the PFA unit 102 calculates impedance (or AO count if an analog to digital converter is being utilized), changes in voltage, etc., and from this calculation, determines the gap between the jaws 2710, 2712 of the PFA device 2700. In the context of using ablation electrode zones, a first zone in step 4206 is powered so diagonal electrodes fire and 800 volts maximum is delivered, a second zone in step 4208 is powered so parallel electrodes are fired and a maximum of 1000 volts is delivered, and in a third zone in step 4210, parallel electrodes are fired with a maximum of 2000 volts. It should be noted that steps 4206, 4208, 4210 may be carried out sequentially, at the same time, or at various non-overlapping times. When the dose is delivered sufficiently to ablate the tissue clamped therebetween for a particular zone, the PFA unit 102 may alert an operator of the PFA device 2700 in the form of an audible, visual, and/or haptic feedback in step 4212.

Referencing FIG. 43, a still further alternate scheme is represented to maximize efficacy, while minimizing the risk of arcing is to use the resolution of the measurement, but operate in three or more tissue ablation zones. In step 4300, the operator establishes communication between the PFA unit 102 and the PFA device 2700 so as to allow the PFA unit 102 to read the memory of the PFA device 2700 to obtain the transfer function or other mathematical relationship information to correlate outputs from the PFA device sensor/meter/switch (2806, 2906, 3032, 3062, 3800) into distance determinations (i.e., jaw spacing). During medical use in step 4302, the PFA device 2700 is utilized to clamp tissue between the jaws 2710, 2712 and energy delivery to the electrodes of the jaws commences based upon zones of the electrodes and tissue to be ablated. In step 4304, the PFA unit 102 calculates impedance (or AO count if an analog to digital converter is being utilized), changes in voltage, etc., and from this calculation, determines the gap between the jaws 2710, 2712 of the PFA device 2700. In the context of using ablation electrode zones, a first zone in step 4306, the PFA unit 102 may calculate the voltage necessary to accomplish a 5000 Volts/centimeter electric field, based on the assumption that 5000 V/cm is the nominal electric field target and 2000 V is the maximum that the PFA device 102 can deliver, and thereafter deliver the necessary energy. In step 4308, parallel electrodes of a second zone are fired with relatively longer pulse widths and more pulses than zone 1, with a 1000 volt maximum. In step 4310, parallel electrodes of a third zone are fired with relatively longer pulse widths and more pulses than zone 1, with a 2000 volt maximum. It should be noted that steps 4306, 4308, 4310 may be carried out sequentially, at the same time, or at various non-overlapping times. When the dose is delivered sufficiently to ablate the tissue clamped therebetween for a particular zone, the PFA unit 102 may alert an operator of the PFA device 2700 in the form of an audible, visual, and/or haptic feedback in step 4312.

In some embodiments involving a clamp-type PFA device, one or more parameters associated with the distance between the jaws may be used to determine, at least in part, one or more PFA energy parameters. For example, one or more PFA energy parameters may be controlled as a function of a parameter associated with a distance between opposed jaws. For example, in one embodiment, the distance between the jaws may be measured and may be used to determine the PFA energy potential (e.g., maximum voltage delivered). In some such embodiments, a greater measured jaw distance may result in a greater PFA energy potential. Accordingly, in some embodiments, a desired cellular transmembrane voltage potential may be achieved across a range of jaw closure distances.

In some example embodiments, at least one PFA energy parameter may increase as a measured parameter increases. In some example embodiments, at least one PFA energy parameter may decrease as a measured parameter decreases. In some example embodiments, at least one PFA energy parameter may increase as a measured parameter decreases. In some example embodiments, at least one PFA energy parameter may decrease as a measured parameter increases.

In some example embodiments, at least one PFA energy parameter may vary substantially linearly with a measured parameter. In some example embodiments, at least one PFA energy parameter may vary substantially non-linearly with a measured parameter.

For example and without limitation, one or more PFA energy parameters, such as those described herein, may be determined and/or may be varied based at least in part upon one or more measurements of voltage, current, inductance, impedance, conductivity, resistance, or temperature.

In some example embodiments according to at least some aspects of the present disclosure, one or more PFA energy parameters, such as those described herein, may be varied based at least in part upon one or more selected parameters. Such selected parameters may be preset when a device or unit is constructed for a particular end use, or may be selected by a user before and/or during use. For example, tissue type, cell density, and/or tissue compression/compressibility may be used to determine, at least in part, one or more PFA energy parameters.

In some example embodiments, two or more measured and/or selected parameters may be used in combination to determine one or more PFA energy parameters. For example, a selected tissue type (which may be associated with known tissue compressibility and/or cell density values, for example), a measured jaw closure distance, and/or a measured jaw closure force may be used, in combination, to determine, at least in part, at least one PFA energy parameter, such as maximum potential. In some embodiments, two or more measured and/or selected parameters may be equally weighted in determining at least one PFA energy parameter. In some embodiments, two or more measured and/or selected parameters may be unequally weighted in determining at least one PFA energy parameter. In some example embodiments, different selected and/or measured parameters may be used to determine, at least in part, different PFA energy parameters. In some example embodiments, different selected and/or measured parameters may be weighted differently in connection with determining, at least in part, different PFA energy parameters.

Generally, any one or more PFA energy parameters described herein may be determined and/or varied based at least in part upon selection and/or measurement of any parameter or condition described herein.

The following patent references may provide context for the present disclosure and are incorporated by reference herein in their entireties: U.S. Pat. No. 9,072,518, issued Jul. 7, 2015, titled “HIGH-VOLTAGE PULSE ABLATION SYSTEMS AND METHODS”; U.S. Pat. No. 9,474,574, issued Oct. 25, 2016, titled “STABILIZED ABLATION SYSTEMS AND METHODS”; U.S. Pat. No. 11,628,007, issued Apr. 18, 2023, titled “CRYOPROBE”; U.S. Pat. No. 10,413,355, issued Sep. 17, 2019, titled “VACUUM COAGULATION PROBES”; U.S. Patent Application Publication No. 2022/0133400, published May 5, 2022, titled “ABLATION DEVICES AND METHODS OF USE”; U.S. Patent Application Publication No. 2019/0159835, published May 30, 2019, titled “CRYOPAD”; and International Application No. PCT/US2022/082057, filed Dec. 20, 2022, published as International Publication No. WO2023129842 on Jul. 6, 2023, titled “MAGNETICALLY COUPLED ABLATION COMPONENTS.” Generally, any features or improvements described herein may be used in connection with embodiments described in these patent references, and any features, elements, or methods described in these patent references may be used in connection with any embodiments described herein.

Unless specifically indicated, it will be understood that the description of any structure, function, and/or methodology with respect to any illustrative embodiment herein may apply to any other illustrative embodiments. More generally, it is within the scope of the present disclosure to utilize any one or more features of any one or more example embodiments described herein in connection with any other one or more features of any other one or more other example embodiments described herein. Accordingly, any combination of any of the features or embodiments described herein is within the scope of this disclosure.

Following from the above description and invention summaries, it should be apparent to those of ordinary skill in the art that, while the methods and apparatuses herein described constitute example embodiments according to the present disclosure, it is to be understood that the scope of the disclosure contained herein is not limited to the above precise embodiments and that changes may be made without departing from the scope as defined by the following claims. Likewise, it is to be understood that it is not necessary to meet any or all of the identified advantages or objects disclosed herein in order to fall within the scope of the claims, since inherent and/or unforeseen advantages may exist even though they may not have been explicitly discussed herein.

What is claimed is:

1. A surgical device, comprising:
a shaft; and
a first jaw and a second jaw operatively coupled to a distal end of the shaft, wherein the first jaw is repositionable with respect to the second jaw;
wherein each of the first jaw and the second jaw comprises at least three electrodes exposed with respect to an insulating material and spaced radially across a longitudinal cross-section thereof;
wherein each of the first jaw and the second jaw terminates at a respective tip.

2. The surgical device of claim 1, wherein:
the longitudinal cross-section of the first jaw includes the insulating material exhibiting a convex profile; and
the longitudinal cross-section of the second jaw includes the insulating material exhibiting a convex profile.

3.-7. (canceled)

8. The surgical device of claim 1, wherein:
the longitudinal cross-section of the first jaw includes the insulating material exhibiting a convex profile; and
the longitudinal cross-section of the second jaw includes the insulating material exhibiting a concave profile.

9.-17. (canceled)

18. The surgical device of claim 1, wherein:
the longitudinal cross-section of the first jaw includes the insulating material exhibiting a concave profile; and
the longitudinal cross-section of the second jaw includes the insulating material exhibiting a concave profile.

19.-41. (canceled)

42. A surgical device, comprising:
a first jaw and a second jaw, wherein the first jaw is repositionable with respect to the second jaw;
wherein each of the first jaw and the second jaw comprises an electrode extending beyond an insulating material;
wherein each of the first jaw and the second jaw terminates at a respective tip; and
wherein at least one of a potentiometer, a Hall effect sensor, and a plurality of continuity switches is operatively coupled to the first jaw and is configured to vary an output voltage as a function of a position of the first jaw with respect to the second jaw.

43. The surgical device of claim 42, wherein at least one of the potentiometer, the Hall effect sensor, and the plurality of continuity switches is configured to be in electrical communication with a pulse frequency ablation controller.

44. The surgical device of claim 42, further comprising:
a handle,
a shaft coupled to the handle, wherein:
the potentiometer is operatively coupled to the first jaw;
the first jaw and the second jaw are operatively coupled to a distal end of the shaft;
a first component of the potentiometer is fixedly mounted to the handle; and
a second component of the potentiometer is fixedly mounted to a repositionable linkage within the handle, the repositionable linkage operatively coupled to the first jaw.

45. The surgical device of claim 44, wherein:
the first component of the potentiometer comprises dual terminals of a resistive element; and,
the second component of the potentiometer comprises at least one of a pogo pin, a cantilever finger, and a spring arm.

46. The surgical device of claim 44, wherein:
the first component of the potentiometer comprises at least one of a pogo pin, a cantilever finger, and a spring arm; and,
the second component of the potentiometer comprises dual terminals of a resistive element.

47. The surgical device of claim 44, wherein the handle houses an actuator mechanism that includes a return spring biasing a housing operatively coupled to the repositionable linkage.

48. The surgical device of claim 47, wherein the actuator mechanism further includes a force limiting spring and a relief rod operatively coupled to the housing.

49. The surgical device of claim 48, wherein the relief rod is operatively coupled to a plunger repositionably mounted to the handle.

50. The surgical device of claim 42, wherein:
the potentiometer is operatively coupled to the first jaw; and
the potentiometer comprises a linear potentiometer.

51. The surgical device of claim 50, wherein the potentiometer comprises a sealed linear membrane potentiometer.

52.-53. (canceled)

54. The surgical device of claim 42, further comprising:
a handle,
a shaft coupled to the handle, wherein:
the Hall effect sensor is operatively coupled to the first jaw;
the first jaw and the second jaw are operatively coupled to a distal end of the shaft;
the Hall effect sensor is fixedly mounted to the handle; and
a magnet is fixedly mounted to a repositionable linkage within the handle, the repositionable linkage operatively coupled to the first jaw.

55. The surgical device of claim 52, wherein:
a magnet is fixedly mounted to the handle; and
the Hall effect sensor is fixedly mounted to a repositionable linkage within the handle, the repositionable linkage operatively coupled to the first jaw.

56. The surgical device of claim 54, wherein the magnet is at least one of a permanent magnet and an electromagnet.

57. The surgical device of claim 54, wherein the handle houses an actuator mechanism that includes a return spring biasing a housing operatively coupled to the repositionable linkage.

58. The surgical device of claim 57, wherein the actuator mechanism further includes a force limiting spring and a relief rod operatively coupled to the housing.

59. The surgical device of claim 58, wherein the relief rod is operatively coupled to a plunger repositionably mounted to the handle.

60.-61. (canceled)

62. The surgical device of claim 42, further comprising:
a handle,
a shaft coupled to the handle, wherein:
the plurality of continuity switches is operatively coupled to the first jaw;
the first jaw and the second jaw are operatively coupled to a distal end of the shaft;
a fixture configured to close at least one of the continuity switches is fixedly mounted to the handle; and
at least one of the plurality of continuity switches is fixedly mounted to a repositionable linkage within the handle, the repositionable linkage operatively coupled to the first jaw.

63. The surgical device of claim 42, further comprising:
a handle,
a shaft coupled to the handle, wherein:
the plurality of continuity switches is operatively coupled to the first jaw;
the first jaw and the second jaw are operatively coupled to a distal end of the shaft;
at least one of the plurality of continuity switches is fixedly mounted to the handle; and
a fixture configured to close at least one of the continuity switches is fixedly mounted to a repositionable linkage within the handle, the repositionable linkage operatively coupled to the first jaw.

64. The surgical device of claim 42, further comprising:
a handle,
a shaft coupled to the handle, wherein:
the plurality of continuity switches is operatively coupled to the first jaw;
the first jaw and the second jaw are operatively coupled to a distal end of the shaft;
a plurality of fixtures, configured to close multiples of the plurality of continuity switches, is fixedly mounted to the handle; and
the plurality of continuity switches are fixedly mounted to a repositionable linkage within the handle, the repositionable linkage operatively coupled to the first jaw.

65. The surgical device of claim 42, further comprising:
a handle,
a shaft coupled to the handle, wherein:
the plurality of continuity switches is operatively coupled to the first jaw;
the first jaw and the second jaw are operatively coupled to a distal end of the shaft;
the plurality of continuity switches are fixedly mounted to the handle; and
a plurality of fixtures, configured to close multiples of the plurality of continuity switches, are fixedly mounted to a repositionable linkage within the handle, the repositionable linkage operatively coupled to the first jaw.

66. The surgical device of claim 62, wherein the handle houses an actuator mechanism that includes a return spring biasing a housing operatively coupled to the repositionable linkage.

67. The surgical device of claim 66, wherein the actuator mechanism further includes a force limiting spring and a relief rod operatively coupled to the housing.

68. The surgical device of claim 67, wherein the relief rod is operatively coupled to a plunger repositionably mounted to the handle.

69. The surgical device of claim 42, wherein:
the plurality of continuity switches is operatively coupled to the first jaw; and
each of the plurality of continuity switches includes it own resistor having a unique resistance.

70.-106. (canceled)

107. A surgical device, comprising:
a first jaw and a second jaw operatively coupled to a distal end of the shaft, wherein the first jaw is repositionable with respect to the second jaw;
wherein each of the first jaw and the second jaw comprises an electrode extending beyond an insulating material;
wherein each of the first jaw and the second jaw terminates at a respective tip; and
wherein a sensor is operatively coupled to the first jaw and is configured to vary an output voltage as a function of a position of the first jaw with respect to the second jaw.
